(19) United States
(12) Patent Application Publication
(10) Pub. No.: US 2009/0304686 A1
(43) Pub. Date: Dec. 10, 2009

(54) INTERFERING IN ACTIVATION OF AN IMMUNE CELL BY INFLUENCING INTERACTION OF LAIR AND COLLAGEN

(75) Inventors: Linde Meyaard, De Bilt (NL); Robert Jan Lebbink, San Francisco, CA (US)

Correspondence Address: MORRISON & FOERSTER LLP, 12531 HIGH BLUFF DRIVE, SUITE 100, SAN DIEGO, CA, 92130-2040, US

(73) Assignees: UNIVERSOTAIR MEDISCH CENTRUM UTRECHT, Utrecht (NL); UNIVERSITEIT UTRECHT, Utrecht (NL)

(21) Appl. No.: 12/281,741

(22) PCT Filed: Mar. 8, 2007
(86) PCT No.: PCT/NL2007/050093

§ 371 Date: May 28, 2009

(30) Foreign Application Priority Data
Mar. 8, 2006 (EP) 06075556.8

(51) Int. Cl. A61K 39/395; C12N 5/02; A61K 38/00; A61K 38/10; A61K 38/16; C12Q 1/02

(52) U.S. Cl. 424/133.1; 435/375; 514/2; 514/12; 514/13; 514/14; 435/29

(57) ABSTRACT

The invention provides a method for interfering in activation of an immune cell, comprising providing a substance which specifically interacts in the binding of leukocyte-associated immunoglobulin-like receptor (LAIR) and collagen. The invention in one aspect provides a method for down regulation of activation of an immune cell. In another aspect the invention provides a method for up regulation of activation of an immune cell. The invention further provides pharmaceutical compositions and uses thereof.

The invention relates to the fields of medicine, immunology and cancer. More in particular the invention relates to intervention in activation of immune cells.

The mammalian immune system has evolved to respond to a large variety of stimuli in order to effectively eliminate pathogens and damaged or malignant cells. For an effective protection of the body, an activation of immune cells is usually required. However, for a balanced and appropriate response, inhibition of activation of immune cells is as important as activation. Inhibition of activation of immune cells is required to terminate an immune response and to prevent excessive immune reactions or autoimmune disease. The immune system is a complex system composed of many activating and many inhibitory components. Important components in the inhibition of activation of immune cells are inhibitory immune receptors. In the past decade, many inhibitory immune receptors have been identified. Most of these receptors contain one or several Immunoreceptor Tyrosine-based Inhibitory Motifs (ITIMs) in the cytoplasmic tail. Binding of a ligand to a receptor comprising an ITIM provides an inhibitory signal by phosphorylation of tyrosine-residues located in the ITIM.

The Leukocyte Associated Immunoglobin-like Receptor-1 (LAIR-1) is an inhibitory immune receptor that contains two ITIM motifs in its cytoplasmatic tail. LAIR-1 is a unique member of the group of inhibitory receptors because of its broad expression in the immune system. LAIR-1 is a member of the Immunoglobin superfamily (IgSF) and is expressed on the majority of peripheral blood mononuclear cells, including natural killer (NK) cells, T-cells, B-cells, monocytes and Dendritic Cells (DCs), as well as the majority of thymocytes. A few years ago the inventors identified a second LAIR molecule, LAIR-2, in humans. LAIR-2 is 84% homologous to LAIR-1. LAIR-2 differs from LAIR-1 in that it lacks a transmembrane and cytoplasmic domain, which indicates a soluble character. In order to identify the position of LAIR in the immune system, it is imperative to delineate the biological function of LAIR. For specification of the biological function of LAIR, the identification of the natural ligand of LAIR is essential. Although immune receptors have been extensively studied in the past decade, the natural ligand of LAIR could so far not be identified. A possible candidate has even been proposed, an Epithelial Cellular Adhesion Molecule (Ep-CAM)³⁰. This molecule was however proven to have been a false candidate as it turned out that the finding had been based on an artefact³¹. The invention provides a high affinity natural ligand of LAIR. The natural ligand of LAIR is, according to the invention shown to be collagen.

Collagens are the most abundant proteins in vertebrates. Collagens are known to play crucial roles in the development, morphogenesis, and growth of many tissues¹. Besides their mechanical properties, collagens serve as substrates for cell attachment, migration, coagulation and mediate signalling events by binding to several cell surface receptors such as integrins, discoidin domain receptors (DDRs), glycoprotein VI, and proteoglycan receptors². The invention provides quite a distinct property of collagen as well, it is a high affinity ligand for the broadly expressed inhibitory Leukocyte Associated Ig-like Receptor (LAIR). Thus far, all documented ligands for immune inhibitory receptors were membrane molecules, implying a regulatory role for these receptors in cell-cell interaction. The finding of the invention that collagens are ligands for an ITIM-bearing receptor shows an entirely other mechanism. This finding of the invention shows a novel mechanism of peripheral immune regulation by extracellular matrix proteins. The invention thus provides an unanticipated role in immunoregulation for collagens. Furthermore, since the invention has identified a high affinity natural ligand of LAIR and hereby specified the biological function of LAIR, it provides a unique mechanism for interference in activation of immune cells.

The invention provides a method for interfering in activation of an immune cell, comprising providing a substance which specifically interacts in the binding of leukocyte-associated immunoglobulin-like receptor (LAIR) and collagen. Interfering as used herein, means any intervening in activation of an immune cell. Interfering thus includes up regulation as well as down regulation. Activation of an immune cell as used in the invention is the process of making an immune cell respond and/or making an immune cell reach a state in which the cell is ready to respond. A substance which specifically interacts in the binding of leukocyte-associated immunoglobulin-like receptor (LAIR) and collagen is any compound that is in any way capable of intervening in the binding of LAIR and collagen. Such a substance is for instance a compound that specifically binds to LAIR or that alternatively specifically binds to collagen. In order to interfere in activation of an immune cell such that said activation is down regulated, a substance is provided that binds and cross-links LAIR. Cross-linking of LAIR provides an inhibitory signal to said immune cell, thereby down regulating activation of said immune cell. A substance that binds and cross-links LAIR is for instance an antibody and/or a peptide with two binding sites. In order to interfere in activation of an immune cell such that said activation is up regulated, a substance is provided that inhibits cross-linking of LAIR. If LAIR is not cross-linked, LAIR does not provide an inhibitory signal to said immune cell, thereby up regulating activation of said immune cell. Said substance that inhibits cross-linking of LAIR is for instance a substance that binds to LAIR without cross-linking LAIR, thereby inhibiting cross-linking of LAIR. Another example of a substance that inhibits cross-linking of LAIR is for instance a substance that binds to collagen, by binding to collagen said substance blocks sites to which LAIR can bind. As LAIR can not bind to collagen and LAIR is not cross-linked, LAIR does not provide an inhibitory signal to said immune cell and thereby activation of said immune cell is up regulated.

An immune cell as used in the invention, reads on any cell that has a role in the realization of an immune response. In a preferred embodiment, said LAIR is part of said immune cell. LAIR-1 is expressed on peripheral blood mononuclear cells, including NK cells, T cells, B cells, monocytes, dendritic cells, thymocytes³, hematopoietic progenitor cells, eosinophils, primed neutrophils¹⁸ and mast cells. Therefore, the invention in a preferred embodiment provides a method for interfering in activation of an immune cell according to the invention, wherein said immune cell comprises an NK cell, a T cell, a B cell, a monocyte, a dendritic cell, a thymocyte, a hematopoietic progenitor cell, an eosinophil, a primed neutrophil and/or a mast cell. The immune cell is any immune cell of an organism, preferably of an animal, more preferably of a vertebrate, more preferably of a mammal, most preferably of a human. A collagen as used in the invention, is defined as including any compound that comprises a collagen molecule or a collagen-like domain whereto LAIR binds. Collagens and proteins with collagen domains form large superfamilies in many species. Vertebrates are known to have at least 27 collagen types with 42 distinct polypeptide chains, more than 20 additional proteins with collagen-like domains and approximately 20 isoenzymes of various collagen-modifying enzymes¹³.

Collagen molecules comprise at least one domain comprising at least one repeat of a G-X-Y repeat, wherein G is glycine and wherein X and Y are amino acid residues. Therefore, the invention in one embodiment provides a method for interfering in activation of an immune cell according to the invention, wherein the substance specifically interacts in the binding of LAIR and a G-X-Y repeat (wherein X and Y are amino acid residues) in collagen. The X and Y in a G-X-Y repeat according to the invention are any amino acid residues. X and Y are preferably any amino residue other than glycine. A proline is however often found in the X position in the repeat in a collagen molecule. Therefore, X is preferably a proline. When X is proline, Y is preferably an amino acid other than proline. In the Y position a hydroxyproline (O) is in many cases present in a G-X-Y collagen repeat. Y is thus preferably a hydroxyproline. In a preferred embodiment the invention provides a method according to the invention, wherein said X is Proline, and wherein said Y is Hydroxyproline. Preferably, a collagen of the invention comprises three polypeptide chains, wherein each chain comprises at least one G-X-Y repeat. Each chain is preferably coiled into a helix, preferably a left-handed helix. More preferably the resulting helices are wound around a common axis to form a triple helix. The presence of glycine, the smallest amino acid, is important for the formation of the triple helix, or coiled-coil, structure. Y is in a preferred embodiment of the invention a 4-hydroxyproline. The 4-hydroxyproline has a predominant role in the stability of the triple helix.

In one aspect of the invention, the invention provides a method according to the invention, wherein LAIR is LAIR-1. LAIR-1 is an important member of the group of LAIR receptors as it is broadly expressed and is present on the majority of peripheral blood mononuclear cells. The invention provides evidence that LAIR-1 is a high affinity collagen receptor. The high affinity character is for instance demonstrated by the fact that the interaction of collagen I and II was of 20-1000 fold higher affinity as compared to most IgSF-members interacting with their ligands¹⁴.

The importance of a well-functioning inhibition mechanism of the immune system is emphasized by the occurrence of auto-immune diseases. In such disorders, the immune system has developed an unwanted immune response against self-antigens. Inhibitory receptors have an important role in prevention of auto-immune diseases. This important role of inhibitory receptors is underlined by receptor knock-out mice which demonstrate enhanced sensitivity to autoimmune-like diseases caused by an over-activated immune system.⁹ In order to at least in part prevent and/or cure an overactive immune response, such as in for instance an autoimmune disorder, it is imperative to interfere in activation of immune cells. The invention provides a method for interfering in activation of an immune cell according to the invention, wherein the interference is a down regulation and the substance is a ligand for LAIR-1. Down regulation as used herein comprises down regulation in quantity and/or in quality compared to a situation without interference. For instance, less immune cells are activated and/or an immune cell is in a less activated state and/or less ready to be activated. The situation without interference is optionally a situation wherein there was no inhibition of the activation of an immune cell. Alternatively, the situation without interference is for instance a situation wherein inhibition of activation of an immune cell was present, for instance due to LAIR-1 stimulation or for example due to stimulation of another inhibitory receptor. A ligand for LAIR-1 is defined as any compound that binds to LAIR-1. Preferably, said ligand by binding to LAIR-1 generates an inhibitory signal to a cell, wherein said cell preferably comprises an immune cell that comprises said LAIR-1. Said inhibitory signal is in a preferred embodiment caused by phosphorylation of tyrosine-residues located in immunoreceptor tyrosine based inhibitory motifs (ITIMs) present in the cytoplasmatic tail of LAIR-1⁷.

In a preferred embodiment the invention provides a method according to the invention, wherein the ligand for LAIR-1 is an anti-LAIR-1 binding body. An anti-LAIR-1 binding body as used in the invention has binding properties specific for LAIR-1. In the art many different specific binding bodies are available. Of old, antibodies are used. However, currently many different parts, derivatives and/or analogues of antibodies are in use as binding bodies Non-limiting examples of such parts, derivatives and/or analogues are, single chain Fv-fragments, monobodies, VHH, Fab-fragments, or artificial binding proteins such as for example avimers, and the like. A common denominator of such specific binding bodies is the presence of an affinity region (a binding peptide) that is present on a structural body that provides the correct structure for presenting the binding peptide. Binding peptides are typically derived from or similar to CDR sequences (typically at least CDR3 sequences) of antibodies, whereas the structure providing body is typically, though not necessarily derived from or similar to framework regions of antibodies. In a preferred embodiment the invention thus provides a method according to the invention, wherein said binding body is an antibody or a functional part, derivative and/or analogue thereof. Therefore, in one embodiment the invention provides a method according to the invention, wherein the ligand for LAIR-1 is an anti-LAIR-1 antibody or a functional part, derivative and/or analogue thereof.

A functional part according to the invention, such as for instance a functional part of a binding body or an antibody according to the invention, is defined as a part that has the same binding properties in kind, not necessarily in amount. A functional derivative according to the invention is defined as a compound which has been altered such that the binding properties of said compound are essentially the same in kind, not necessarily in amount. The invention further provides derivatives according to the invention, for instance a derivative of a binding body, antibody, protein or peptide according to the invention. A derivative can be provided in many ways. A derivative of a protein or peptide is for instance provided through conservative amino acid substitution. A person skilled in the art is further well able to generate analogues. An analogue of a protein or peptide is for instance generated through screening of a peptide library. Such an analogue has essentially the same immunogenic properties of said protein or peptide in kind, not necessarily in amount. An analogue of an antibody for instance, has essentially the same binding properties of said antibody in kind, not necessarily in amount. An example of an analogue of an antibody is an avimer.

Since the invention provides an unanticipated role for collagens in immunoregulation, the invention provides a novel mechanism for interfering in the immunoregulation. The invention provides a method for interfering in activation of immune cells by providing a collagen. The invention in one embodiment provides a method for interfering in activation of an immune cell according to the invention, wherein the ligand for LAIR-1 comprises a proteinaceous part, preferably a protein, more preferably a peptide comprising several G-X-Y repeats. Several repeats as used in the invention, are a number of repeats in said ligand that enables said ligand of cross-linking LAIR-1. Cross-linking of LAIR-1 delivers a potent inhibitory signal that is capable of inhibiting cellular functions of NK cells, T cells, B cells, monocytes and dendritic cells³⁻⁶. A person skilled in the art is well equipped to assess an optimal number of repeats in a ligand. In a preferred embodiment of the invention, the number of repeats is established in order to effectively cross-link LAIR. In this embodiment a inhibitory signal is delivered to an immune cell and such a number of repeats is thus preferably used in a method for interfering in activation of an immune cell according to the invention, wherein the interference is a down regulation. There is a limit to the number of repeats in a ligand in order for the ligand to be effective in cross-linking LAIR. Said number of repeats in said ligand in order to cross-link LAIR is at least one, more preferably at least 6, more preferably at least 8, most preferably at least 10. Said number of repeats is preferably less than 200, more preferably less than 100, more preferably less than 50, most preferably less than 25. Said ligand comprising said number of repeats binds to LAIR and thereby LAIR is cross-linked. In an alternative embodiment of the invention, the number of repeats is established in order to inhibit cross-linking of LAIR and thus to prevent deliverance of an inhibitory signal to an immune cell. This embodiment is therefore specifically suitable for a method for interfering in activation of an immune cell according to the invention, wherein the interference is an up regulation. In this embodiment said number of repeats in said ligand in order to inhibit cross-linking of LAIR is preferably at least 12, more preferably at least 18, more preferably at least 25, most preferably at least 30. Said number of repeats is preferably less than 200, more preferably less than 100, more preferably less than 75, most preferably less than 50. Said ligand comprising said number of repeats binds to LAIR and thereby inhibits cross-linking of LAIR. In order to preserve a collagen-like structure, the repeats should preferably have a distance that mimics absolute or relative distances that is present between any repeats in collagen. An optimal number of repeats should preferably be assessed by the person skilled in the art in coordination with the assessment of the optimal distance between the repeats. In a preferred embodiment the invention provides a method for interfering in activation of immune cells according to the invention, wherein said G-X-Y repeats comprise several G-P-O repeats. In a further preferred embodiment the invention provides a method according to the invention, wherein the ligand for LAIR-1 comprises a peptide having several G-X-Y repeats, wherein said peptide forms a triple-helical peptide. In one embodiment of the invention a G-X-Y repeat is a triple helical peptide comprising 10 repeated GPO triplets, (GPO)₁₀. Such a (GPO)₁₀ is also known as collagen-related peptide (CRP).

The invention provides extracellular matrix collagens as functional ligands for the inhibitory LAIR-1 that can directly down regulate immune responses. Herein underneath a non-limiting theory of a natural immunoregulation mechanism is described. When immune cells migrate into the tissues they are potentially exposed to multiple activating signals. To ensure that immune cells respond adequately to these stimuli, inhibitory receptors are required to set a threshold for cell activation⁸. The results presented herein show that collagen/LAIR-1 interactions can inhibit cell activation and as such contribute to a dampening of the response. Under physiological conditions, immune cells present in the blood are not exposed to collagens¹⁷. Their extravasation however, results in interaction with collagen-rich sub endothelial structures, which increase the threshold for immune cell activation needed to keep these potentially dangerous cells in check. When immune cells reach an inflammatory locus, the presence of specific and strong activating stimuli given by antigen-presenting cells, cytokines or pathogens, will override the threshold and allow immune cells to become activated and conduct their function. Indeed, it was observed in the invention that sub-optimal activation via the FcεR could be efficiently downregulated via the LAIR-1/collagen interaction, whereas maximal activation could not (data not shown). Regulation of the LAIR-1/collagen interaction can also occur by modulating LAIR-1 expression at different stages of differentiation/activation of immune cells, as was previously demonstrated for B cells⁴, T cells⁶, neutrophils¹⁸, and dendritic cells (unpublished observations of the inventors).

Depletion of inhibitory immune receptors or down regulation of ligands for these receptors can result in an hyper-activated immune system leading to chronic inflammation and autoimmune-like phenotypes⁹. Collagens are implied in various human (autoimmune-like) diseases. Collagen XVII for instance, is identified as an auto antigen in acquired blistering disorders like bullous pemphigoid¹⁹. Collagens II and VII are autoantigens in rheumatoid arthritis and systemic lupus erythematosus (SLE)^{20;21} and epidermolysis bullosa acquisita²² respectively. Autoantibodies targeting the various collagen molecules can interfere with the collagen-LAIR-1 interaction and as such play a role in the pathology of these diseases. The invention therefore in one embodiment provides a method for interfering in activation of immune cells according to the invention, to at least in part prevent, ameliorate and/or cure an auto-immune disease in an individual. Ameliorate as used herein, is defined as comprising at least reducing an intensity and/or an extent of at least one of the symptoms of said auto-immune disease in said individual. An auto-immune disease often involves an immune response raised against at least one auto-antigen. Alternatively, in other autoimmune diseases the immune system is inappropriately activated. An auto-antigen is defined as a normal part of the body against which no significant immune response is raised in a healthy individual. Said autoimmune disease comprises for instance rheumatoid arthritis, scleroderma and systemic lupus erythematosus (SLE). There is at present no effective preventive or curing medicament available against said autoimmune diseases. Therefore, in these cases it is highly desirable to interfere in activation of an immune cell with a method according to the invention. Interfering in activation of immune cells according to the invention, to at least in part prevent, ameliorate and/or cure an auto-immune disease in an individual, comprises down regulating activation of an immune cell. Thus, a method for interfering in activation of an immune cell according to the invention, wherein the interference is a down regulation, is suitable. In order to provide down regulation of activation of said immune cell, a substance is provided that binds to and cross-links LAIR. Cross-linked LAIR than provides an inhibitory signal to said immune cell. Said substance in this embodiment thus comprises a ligand for LAIR, for instance an anti-LAIR-1 antibody or a functional part, derivative and/or analogue thereof, and/or a peptide comprising several G-X-Y repeats, preferably several G-P-O repeats, wherein said peptide preferably forms a triple-helical peptide. In a preferred embodiment, said peptide comprising several G-X-Y repeats has a length of at least 15 and at most 50 amino acid residues, preferably a length of at least 20 and at most 40 amino acid residues, and comprises a sequence which is at least 80% homologous to at least part of the sequence GPMGPMGPRGPOGPAGAOGPQGFQGNO, GTOGTDGPKGASGPAGPOGAQGPOGLQ, GRAGEOGLQGPAGPOGEKGEOGDDGPS, GAOGAOGPOGSOGPAGPTGKQGDRGEA, GPRGRSGETGPAGPOGNOGPOGPOGPO, GLAGYOGPAGPOGPOGPOGTSGHOGSO, GERGLOGPOGIKGPAGIOGFOGMKGHR, GAOGLRGGAGPOGPEGGKGAAGPOGPO, GMOGERGGLGSOGPKGDKGEOGGOGAD, GEGGPOGVAGPOGGSGPAGPOGPQGVK, GAOGPLGIAGITGARGIAGPOGMOGPR, GPOGMOGPRGSOGPQGVKGESGKOGAN and/or GPAGPAGAOGPAGSRGAOGPQGPRGDK, said part having at least 15 amino acid residues. As disclosed in the Examples, these sequences are particularly well capable of binding LAIR and downregulating activation of immune cells. Said peptide preferably comprises a sequence which is at least 85%, more preferably at least 90%, most preferably at least 95% homologous to at least part of said sequences, said part having at least 15 amino acid residues. In one embodiment said peptide comprising several G-X-Y repeats consists of one of the above cited sequences.

In a particularly preferred embodiment a peptide is used with a length of at least 15 and at most 50 amino acid residues, preferably a length of at least 20 and at most 40 amino acid residues which comprises a sequence which is at least 80%, preferably at least 85%, more preferably at least 90%, most preferably at least 95% homologous to at least part of the sequence GAOGLRGGAGPOGPEGGKGAAGPOGPO, GPRGRSGETGPAGPOGNOGPOGPOGPO, GTOGTDGPKGASGPAGPOGAQGPOGLQ, GEGGPOGVAGPOGGSGPAGPOGPQGVK, GRAGEOGLQGPAGPOGEKGEOGDDGPS and/or GPAGPAGAOGPAGSRGAOGPQGPRGDK, said part having at least 15 amino acid residues. These sequences are preferred for binding LAIR and downregulating activation of immune cells. In one embodiment said peptide comprising several G-X-Y repeats consists of the sequence

GAOGLRGGAGPOGPEGGKGAAGPOGPO,
GPRGRSGETGPAGPOGNOGPOGPOGPO,
GTOGTDGPKGASGPAGPOGAQGPOGLQ,
GEGGPOGVAGPOGGSGPAGPOGPQGVK,
GRAGEOGLQGPAGPOGEKGEOGDDGPS
and/or
GPAGPAGAOGPAGSRGAOGPQGPRGDK.

In another aspect the invention provides a method for interfering in activation of an immune cell according to the invention, wherein the interference is an up regulation and the substance is a specific binding body for LAIR. Preferably said binding body comprises 1 antigen-binding site. Up regulation as used in the invention comprises up regulation in quantity and/or in quality compared to a situation without interference. For instance, more immune cells are activated and/or an immune cell is in a more activated state and/or more ready to be activated. The situation without interference is optionally a situation wherein there was no up regulation of the activation of an immune cell. Alternatively, the situation without interference is for instance a situation wherein up regulation of activation of an immune cell was present, for instance due to blockage of an inhibitory signal of LAIR-1 and/or due to blockage of an inhibitory signal of another inhibitory receptor, but to a lesser degree. A binding body comprising 1 antigen-binding site is for instance a single chain fragment of an antibody or a Fab fragment or a functional part, derivative and/or analogue thereof. A binding body for LAIR in a method according to the invention, wherein the interference is up regulation binds to LAIR without cross-linking the inhibitory receptor. Because said binding body does not cross-link LAIR, the receptor is blocked. The receptor will therefore not provide an inhibitory signal.

A method according to the invention for interfering in activation of an immune cell, wherein the interference is an up regulation, is beneficial in any case wherein an active immune response is needed. The active immune response is for instance required against pathogens and malignant cells. An immune response is in one aspect a normal functioning immune response wherein activation of immune cells is enhanced. Alternatively, an immune response is an inhibited immune response, for instance by action of inhibitory immune receptors. Although inhibitory immune receptors are indispensable for the immune system to prevent excessive activation and auto-immunity, there is a drawback to the inhibition mechanism provided by these receptors. Pathogens and malignant cells can use the inhibitory immune receptors to their advantage and escape from an active immune system. Therefore, the invention provides a method for interfering in activation of an immune cell, wherein the interference is an up regulation according to the invention, to at least in part prevent and/or cure a viral infection or a tumour in an individual. The role of collagen in modulation of an immune response as found by the invention can further explain why expression of several members of the collagen family, such as collagen I, III, V, VI, XIII, XVII, XVIII, XXIII by neoplastic cells, is associated with tumor progression²⁴⁻²⁸. Overexpression of collagens by tumour cells can enable these cells to suppress anti-tumour responses via the immune inhibitor LAIR-1. The principal collagen species in primary tumours are the transmembrane collagens as there is little extracellular collagen present within non-invasive tumours. For invasive and/or metastatic tumours an extracellular collagen, such as collagen I, is of importance as well.

In one embodiment the invention provides a method according to the invention for interfering in activation of an immune cell, wherein the interference is up regulation and the substance is a specific binding body for collagen. Such a specific binding body for collagen occupies sites of collagen to which LAIR binds. When a binding body is bound to such a site, LAIR can not bind and cross-link and thus an inhibitory signal of LAIR is prevented. For instance, if an immune cell comprising LAIR encounters collagen whereto specific binding bodies are bound, the chance of LAIR binding to said collagen is at least diminished. Therefore, most immune cells encountering the collagen do not receive an inhibitory signal of LAIR and thereby up regulation of activation of an immune cell results. In a preferred embodiment of the invention, a method according to the invention is provided for interfering in activation of an immune cell, wherein the interference is up regulation and the substance is a specific binding body for collagen wherein the specific binding body binds to a G-X-Y repeat. In particular said G-X-Y repeat is a G-P-O repeat. In a further preferred embodiment the invention provides a method according to the invention for interfering in activation of an immune cell, wherein the interference is up regulation and the substance is a specific binding body for collagen, and wherein said specific binding body is an antibody or a functional part, derivative and/or analogue thereof, preferably a human or humanised antibody or a functional part, derivative and/or analogue thereof against a collagen. A humanised antibody as used herein is defined as any antibody, derived from an at least not entirely human source, that is made to be less immunogenic in a human. Such an antibody is for instance derived by grafting of complementarity-determining regions (CDRs) from an at least not entirely human source, onto the framework of a human immunoglobulin molecule. Alternatively or additionally, a humanised antibody is derived by selectively or non-selectively removing epitopes that are antigenic in a human from an antibody.

In one embodiment the invention provides a method according to the invention for interfering in activation of an immune cell, wherein the interference is up regulation and the substance is a specific binding body for collagen, wherein said specific binding body is a secreted LAIR or a functional part, derivative and/or analogue thereof A secreted LAIR has a high affinity for collagen and can thus efficiently occupy binding sites of collagen whereto LAIR-1 can bind. Therefore immune cells comprising LAIR-1 can not bind and do not receive an inhibitory signal. A secreted LAIR as used in the invention is preferably LAIR-2. In an alternative embodiment, the invention provides a method according to the invention for interfering in activation of an immune cell, wherein the interference is up regulation and the substance is a specific binding body for collagen, wherein said specific binding body is a soluble LAIR or a functional part, derivative and/or analogue thereof. A soluble LAIR comprises any soluble LAIR component that has the same binding specificity in kind, not necessarily in amount. A soluble LAIR for instance comprises a recombinant LAIR, preferably a recombinant LAIR-1. Binding of collagen by secreted LAIR-2 or a soluble LAIR, can thus serve as a regulator of LAIR-1 function by binding collagen and thus circumventing the inhibitory potential of LAIR-1. Preferred collagens as used in the invention are collagens I, II, III, XIII, XVII, or XXIII. Therefore, in one embodiment the invention provides a method according to the invention, wherein said collagen is collagen I, II, III, XIII, XVII, or XXIII.

A functional part of a LAIR is defined as a part which has at least one same property as LAIR in kind, not necessarily in amount. Said functional part is capable of binding collagen, albeit not necessarily to the same extent. A functional derivative of LAIR is defined as a LAIR which has been altered such that at least one property≧preferably a collagen-binding property and the capability of downregulating activation of an immune cell—of the resulting compound is essentially the same in kind, not necessarily in amount. A derivative is provided in many ways, for instance through conservative amino acid substitution, whereby an amino acid residue is substituted by another residue with generally similar properties (size, hydrophobicity, etc), such that the overall functioning is likely not to be seriously affected.

A person skilled in the art is well able to generate analogous compounds of LAIR. This is for instance done through screening of a peptide library. Such an analogue has essentially at least one same property as LAIR in kind, not necessarily in amount.

Binding bodies and peptides according to the invention, are suitable for interfering in activation of an immune cell. In one aspect the invention provides pharmaceutical compositions comprising peptides and/or binding bodies. In one embodiment the invention provides a pharmaceutical composition comprising a peptide having G-X-Y repeats, wherein said G-X-Y repeats are preferably G-P-O repeats. A pharmaceutical composition according to the invention further preferably comprises an additive such as a suitable carrier. In a preferred embodiment the invention provides a pharmaceutical composition according to the invention, wherein said peptide forms a triple-helical peptide. In a further preferred embodiment the invention provides a pharmaceutical composition comprising a human or humanised antibody or a functional part, derivative and/or analogue thereof against collagen and preferably a suitable carrier. In a further aspect the invention provides a pharmaceutical composition comprising a secreted LAIR or a functional part, derivative and/or analogue thereof, wherein said secreted LAIR is preferably LAIR-2, and wherein said pharmaceutical composition further preferably comprises a suitable carrier. Also, the invention provides a pharmaceutical composition comprising a soluble LAIR or a functional part, derivative and/or analogue thereof, wherein said soluble LAIR is preferably LAIR-1, and wherein said pharmaceutical composition further preferably comprises a suitable carrier. Further provided is the use of a peptide having G-X-Y repeats, wherein said G-X-Y repeats are preferably G-P-O repeats, for the preparation of a pharmaceutical composition for interfering in activation of an immune cell. Said interference is preferably a down regulation to at least in part prevent and/or cure an auto-immune disease in an individual. In another embodiment the invention provides the use of a human or humanised antibody or a functional part, derivative and/or analogue thereof against collagen, for the preparation of a pharmaceutical composition for interfering in activation of an immune cell. Said interference is preferably an up regulation to at least in part prevent and/or cure an infection (for instance a viral infection) or a tumour in an individual. Also provided is a secreted LAIR, preferably LAIR-2 or a functional part, derivative and/or analogue thereof, for use as a medicament. In a further embodiment the invention provides the use of a secreted LAIR or a functional part, derivative and/or analogue thereof, wherein said secreted LAIR is preferably LAIR-2, for the preparation of a pharmaceutical composition for interfering in activation of an immune cell. Preferably a pharmaceutical composition for upregulating an immune response is prepared. Said pharmaceutical composition is preferably capable of at least in part preventing, ameliorating and/or curing an infection and/or a tumor-related disease.

Further provided is a soluble LAIR, preferably LAIR-1 or a functional part, derivative and/or analogue thereof, for use as a medicament. In another embodiment the invention provides the use of a soluble LAIR or a functional part, derivative and/or analogue thereof, wherein said soluble LAIR is preferably LAIR-1, for the preparation of a pharmaceutical composition for interfering in activation of an immune cell. Said interference is preferably an up regulation of an immune response. In one preferred embodiment said pharmaceutical composition is capable of at least in part preventing, ameliorating and/or curing an infection and/or a tumor-related disease. Further provided is a method for at least in part preventing, ameliorating and/or curing an infection and/or a tumor-related disease, comprising administering a secreted LAIR and/or a soluble LAIR, preferably LAIR-2 and/or LAIR-1, or a functional part, derivative and/or analogue of LAIR-2 or LAIR-1, to a subject suffering from, or at risk of suffering from, an infection and/or a tumor-related disease.

As already explained, LAIR-2 is capable of binding collagen and can thus efficiently occupy binding sites of collagen whereto LAIR-1 can bind. Hence, when LAIR-2 has bound collagen, immune cells comprising LAIR-1 cannot bind and do not receive an inhibitory signal. Hence, in the presence of a compound capable of decreasing the amount and/or activity of LAIR-2, more immune cells comprising LAIR-1 are capable of binding collagen. These immune cells will receive an inhibitory signal. The invention therefore also provides a use of a compound capable of decreasing the amount and/or activity of LAIR-2 for the manufacture of a medicament for downregulating an immune response. Said medicament is preferably capable of at least in part preventing, ameliorating and/or curing an auto-immune disease. In one particularly preferred embodiment said compound comprises an anti-LAIR-2 antibody or a functional part, derivative and/or analogue thereof.

A method for at least in part preventing, ameliorating and/or curing an auto-immune disease comprising administering a compound capable of decreasing the amount and/or activity of LAIR-2 to a subject suffering from, or at risk of suffering from, an auto-immune disease is also provided. Said compound preferably comprises an anti-LAIR-2 antibody or a functional part, derivative and/or analogue thereof

According to the present invention, LAIR-2 is present during inflammation. Further provided is therefore a method for determining whether an immune response in an individual is upregulated, comprising measuring the amount of LAIR-2 is a sample of said individual and determining whether said amount is indicative for an upregulated immune response. Said sample preferably comprises a urine sample.

The invention is further illustrated by the following examples. The examples are not to be interpreted as limiting the scope of the invention in any way.

### EXAMPLES

### Example 1

### Materials and Methods

Cells, Transfectants and cDNA.

All cells were obtained from American Type Culture Collection. cDNA encoding human LAIR-1a and mouse LAIR-1a was cloned into the pMX-neo retroviral vector. Full-length mouse collagen XXIII was PCR-amplified from mouse lung cDNA and cloned in-frame with a C-terminal His₆- and FLAG-tag in the pCEP4-vector. Full-length human collagen XVII, HIS-tagged human collagen XIII, and human KIR3DL1 were kindly provided by PA Khavari (Stanford University, USA), T Väisänen (University of Oulu, Finland) and L L Lanier (UCSF, USA) respectively.

Retroviral-based constructs were packaged using the pCL-eco or pCL-ampho system²⁹ and virus was used to infect Ba/F3 or K562 cells. Three days after transduction, transfectants expressing either hLAIR-1, mLAIR-1, human collagen XVII, or human KIR3DL1 were sorted using a flow cytometer (FACSAria; BD Biosciences) for high expression. Human collagen XVII expression was assessed using the 233 monoclonal antibody (a kind gift from K. Owaribe, Nagoya University, Japan). Mouse LAIR-1 was detected using a biotinylated anti-mouse LAIR-1 monoclonal antibody.

### Detection of LAIR Ligand.

Chimeric proteins of rat, mouse, and human LAIR-1 or human LAIR-2 fused to the Fc region of human IgG1 were prepared and cell lines were stained with these reagents in the absence or presence of blocking antibodies as described previously¹¹. When indicated, cells were incubated for 1 h with 100 units/ml chromatography purified C. histolyticum collagenase Type VII (Sigma) prior fusion-protein staining.

### Conjugate-Analysis.

K562 transfectants were labeled for 5 min at room temperature with either PKH67 (green) or PKH26 (red) (Sigma) according manufacturers protocol. Cells were mixed at a ratio of 1:1 and incubated at 37° C. for 1.5 h for mLAIR-1-expressing cells and 5 h for hLAIR-1-expressing cells. Cells were gently resuspended before flow cytometric analysis or analyzed by light microscopy.

### Analysis of the Binding of K562 Transfectants to Plate-Bound Collagens.

Purified commercial collagens I, III (Sigma), II (Chemicon International), or BSA (100 μl/well, 20 μg/ml in PBS, 2 mM acetic acid) were coated overnight at 4° C. in 96-well MAXIsorp (Nunc) flat-bottom plates. After washings, wells were blocked with 1% (w/v) BSA. Meanwhile, 5×10⁶ cells/ml wild type K562 cells or K562 transfectants were fluorescently labeled for 30 min at 37° C. with 5 μM calceine AM (Molecular Probes) in PBS. Cells were washed twice with RPMI/1% FCS and 100 μl medium of 1.5×10⁶ cells/ml cells was added to each well and plates were incubated at 37° C. for 2 h. Input fluorescence was determined using a fluorescence plate reader (Fluoroskan Ascent, Thermo Labsystems), the plates were firmly flicked and washed for six times in culture medium. The retained fluorescence was determined for each well as a percentage of input fluorescence.

### Perfusion Studies.

Perfusions were carried out in a single-pass perfusion chamber as described previously¹⁶. Briefly, collagen type III (Sigma) was solubilized in 50 mM acetic acid and sprayed on glass coverslips using a retouching airbrush (Badger model 100; Badger Brush) at a density of 6.5 μg/cm². Afterwards, coverslips were blocked for 1 h at room temperature with 1% human albumin in PBS.

Subsequently, wild type or transfected K562 cells were perfused for 5 min at a shear rate of 0.75 dyne/cm² at 37° C. After perfusion, slides were washed with HEPES buffer (10 mm HEPES, 150 mm NaCl, pH 7.35), fixed in 0.5% glutaraldehyde in PBS, dehydrated in methanol and stained with May-Grünwald and Giemsa. Adhered K562 cells were counted using light microscopy and presented as number of cells per mm².

### Results

### Collagen XVII is a Ligand for LAIR-1.

By expression cloning we identified transmembrane collagen XVII as a ligand for LAIR-1 (FIG. S1, for methods: see supplemental material). The interaction was confirmed by specific binding of human, mouse and rat LAIR-1-IgG fusion proteins to Ba/F3 cells stably transfected with human collagen XVII (FIG. 1A). Binding of hLAIR-1-IgG and mLAIR-1-IgG to human collagen XVII was blocked by anti-hLAIR-1 Abs (8A8) or polyclonal anti-mLAIR-1 Abs respectively, demonstrating the specificity of these interactions (FIGS. 1B and 1C respectively). The association was divalent cation-independent since presence of EDTA did not affect LAIR-1 fusion-protein binding (data not shown). In addition, human LAIR-2, a putatively secreted protein that is 84% homologous to human LAIR-1¹⁰, interacted with human collagen XVII (FIG. 1A). Thus, collagen XVII is a ligand for LAIR-1 and LAIR-2 and, as we observed previously, ligand recognition occurs cross-species^{11;12}. To confirm that LAIR-1 expressed on cells can bind to collagen XVII, we measured formation of conjugates between LAIR-1 and collagen XVII transfected K562 cells by 2-color flow cytometry after co incubating cells at 37° C. We observed profound aggregation between LAIR-1 and collagen XVII-expressing cells, an interaction that was formed within minutes and remained stable for at least 24 hours (FIGS. 2A and 2B).

LAIR-1 is a General Collagen Receptor

We next investigated whether the previously observed binding of LAIR-1 to human tumor cell lines¹¹ correlated with collagen XVII expression. LAIR-1-ligand-positive¹¹ HT29 colon carcinoma cells expressed collagen XVII and pre-treatment of these cells with C. histolyticum collagenase abrogated both LAIR-1-IgG and collagen XVII mAb-staining (FIG. 3A). The collagen XVII-lacking breast carcinoma cell line SK-BR-3 however, also expressed a ligand for LAIR-1 that was removed after collagenase treatment, suggesting that LAIR-1 binds another collagen family member on these cells (FIG. 3A). Indeed, transient expression of transmembrane collagens XIII and XXIII in 293T cells resulted in binding of LAIR-1-IgG fusion proteins (FIG. 3B). In addition, immobilized non-transmembrane collagens I, II and III were ligands for mouse and human LAIR-1 (FIG. 3C and data not shown). LAIR-1-transfected K562 cells firmly adhered to collagens, which coincided with cell spreading (FIG. 3D). Both mouse and human LAIR-1-IgG immunoprecipitated hcollagen III from solution (FIG. 3E). Furthermore, insoluble collagen I fibrils specifically precipitated LAIR-1 from hLAIR-1-transfected K562 cell-lysate as well as from human PBMC-lysate expressing endogenous LAIR-1 (FIG. 3F). Additionally, mouse and human LAIR-1-IgG bound specifically to human skin tissue sections (FIG. 3G). The collagen I and III-rich dermis stained brightly with LAIR-1-IgG, which was completely blocked by pre-incubation of the fusion proteins with hcollagen I. We conclude that LAIR-1 is a general collagen receptor.

LAIR-1 is a High-Affinity Collagen Receptor

We measured affinity of the collagen-LAIR interaction by surface plasmon resonance (BIAcore) experiments (for methods see supplemental materials). Human LAIR-1 fusion proteins bound with high affinity to collagen I and III (FIG. 4A) and its relatively slow dissociation was characterized by a rapid initial phase and a slower secondary phase (FIG. 4B). The interaction was of 20-1000 fold higher affinity as compared to most IgSF-members interacting with their ligands¹⁴. A single triple-helical collagen I or III molecule interacted with approximately 10.4 and 9.7 LAIR-1 molecules respectively (FIG. 4B) and this high affinity interaction was sufficient to arrest K562 cells expressing human LAIR-1 on collagen III-coated coverslips under flow conditions (FIG. 4C).

LAIR-1 Binds Gly-Pro-Hyp Collagen Repeats

Since LAIR-1 interacted cross-species with various groups of collagen molecules, we hypothesized that LAIR-1 bound to a common collagen-restricted structure. The collagen super family is a large family of trimeric molecules composed of three polypeptide α chains, which contain the sequence repeat (Gly-X-Y)ₙ, X being frequently proline (P) and, after post-translational modification, Y being hydroxyproline (O). The GPO triplet is almost exclusively present in collagenous molecules and allows the formation of a triple helix, which is the main characteristic feature of collagens¹⁵. Immobilized triple-helical peptides composed of 10 repeated GPO triplets ((GPO)₁₀, also known as collagen-related peptide, CRP¹⁶) bound hLAIR-1-IgG (FIG. 4D), whereas a corresponding triple-helical (GPP)₁₀ peptide did not. Thus LAIR-1 binds a common collagen motif in a hydroxyproline-dependent manner. hLAIR-1-IgG bound less efficient to (GPO)₁₀ peptide as compared to collagen, suggesting that, apart from the GPO-sequence, additional structural components can provide a more optimal interaction.

Collagens Directly Cross-Link LAIR-1 and Inhibit Degranulation of RBL-2H3 Cells via LAIR-1

To analyze whether collagen induces functional cross-linking of LAIR-1, we generated 2B4 NFAT-GFP reporter cells³² expressing a chimeric protein consisting of the extracellular domain of hLAIR-1 and the transmembrane and intracellular domain of CD3ζ. Receptor engagement of cells expressing the hLAIR-1-CD3ζ chimera, but not the parental cells, via plate-bound collagens I, III, or anti-hLAIR-1 mAbs resulted in expression of GFP (FIG. 5A). Pre-treatment of reporter cells with anti-hLAIR-1 F(ab′)₂ fragments abrogated the NFAT-activation (data not shown). Collagens I and III are thus capable of cross-linking hLAIR-1.

We next investigated whether cross-linking of LAIR-1 by extracellular matrix collagens leads to inhibition of immune cell function in vitro. As a model, we used LAIR-1 transfected RBL-2H3 cells, which express endogenous IgE receptor FcεRI⁷. Incubation of RBL-2H3 hLAIR-1 transfectants with TNP-specific IgE and subsequent triggering with plate-bound TNP-conjugated BSA resulted in degranulation of the cells and release of β-glucuronidase (FIG. 5B). Simultaneous cross-linking of hLAIR-1 using plate-bound anti hLAIR-1 mAb, collagen I or III caused marked inhibition of degranulation (FIG. 5B). A LAIR-1 mutant that is unable to signal, because the tyrosine residues in the ITIM were changed to phenylalanine (LAIR-1-FF)⁷, could not inhibit the degranulation upon collagen interaction (FIG. 5C). Furthermore, plate-bound triple-helical (GPO)₁₀ alone, but not (GPP)₁₀, was capable of specifically inhibiting the degranulation of RBL cells (FIG. 5D), showing that other GPO-repeat-bearing collagens also inhibit immune cell function by binding to LAIR-1. The effect was specifically due to the LAIR-1/collagen interaction since pre-incubation of the cells with blocking anti-hLAIR-1 F(ab′)₂ fragments completely abolished the inhibition (FIGS. 5B and 5D). Thus, extracellular matrix collagens are functional ligands for the inhibitory LAIR-1 that directly down regulate immune responses.

Supplemental Materials and Methods

Identification of LAIR-Ligand

To identify the natural ligand for LAIR-1 we employed expression cloning using a mouse retroviral cDNA library (day 14 whole embryo, a gift from GQ Daley, Harvard Medical School, Boston, Mass., USA). Viral supernatant was produced¹ and used to infect LAIR-ligand-negative Ba/F3 cells. Three days after transduction, LAIR-ligand positive cells were sorted in the presence of 20% normal mouse serum using magnetic beads (Dynal, Oslo, Norway) coated with mLAIR-1-IgG, allowed to amplify, and subsequently cloned by limiting dilution. We obtained 57 LAIR-ligand positive clones from two independent transductions (example: FIG. S1A). PCR amplification of the retroviral inserts using primers specific to the cDNA's flanking regions that was present in the retroviral expression vector, was not successful, most likely due to the large size of the insert. We took two alternative approaches to reveal the identity of LAIR-ligand in these clones. First, we compared the mRNA expression of two independent LAIR-ligand-expressing Ba/F3 clones with the parental Ba/F3 line using microarrays. Briefly: RNA was extracted using the TRIzol reagent according the procedure recommended by the supplier (Invitrogen). RNA amplification and labeling of cRNA was performed as described² and hybridized onto Corning UltraGAPS slides containing the Operon Mouse Genome Oligo Set V3³. After washing, scanning and data-extraction, analysis was performed with SAM 1.2.1⁴ and/or ANOVA (R/MAANOVA version 0.95-3). This screen revealed two mRNAs that were significantly upregulated in both mouse LAIR-ligand positive clones compared to the control line. These encoded the transmembrane collagen XVII and an unknown putative protein. In parallel we performed immunoprecipitation, using mLAIR-1-IgG on surface biotinylated LAIR-ligand-expressing cells and the parent Ba/F3 cells. Briefly: protein A/G PLUS-agarose beads (Santa Cruz Biotechnology) were coated with mLAIR-1-IgG fusion proteins. 2×10⁹ Ba/F3 cells were surface-biotinylated for 30 min on ice using 2 mg/ml Biotin 3-sulfo-N-hydroxysuccinimide ester sodium salt (Sigma) in PBS and washed three times with 10 mM ammonium-chloride in PBS. Cells were subsequently lysed in Triton lysis buffer (10 mM Tris-HCl, pH 7.5, 150 mM NaCl, 1% Triton X-100 and 0.02% sodium azide) supplemented with protease inhibitors (Complete Mini EDTA-free protease inhibitor cocktail tablets; Roche) and 1 mM phenylmethylsulfonyl fluoride. Cell lysates were cleared by centrifugation and used for immunoprecipitation for 2 h in the presence of 0.5% BSA. Beads were washed 5 times with Triton wash buffer (10 mM Tris-HCl, pH 7.5, 150 mM NaCl, 0.1% Triton X-100 and 0.02% sodium azide). Interacting proteins were subsequently dissolved in electric focusing (IEF)-buffer (8M urea, 2M Thiourea, 4% CHAPS, 20 mM DTT, 0.2% Biolythe pH3-10 and 0.2% broomphenolblue) and subjected to 2D-electrophoresis using Immobiline dry strips (pH 3-10; 11 cm) on an Ettan™ IPGphor™ system (Amersham, Little Chalfont, UK) for separation in the first dimension. Subsequently, separation in the second dimension was performed on 10% polyacrylamide gels SDS-PAGE using a Sturdier Vertical Slab Gel Unit (Hoefer Scientific Instruments, San Francisco, Calif., USA). The gels were transferred to Immobilon-P membranes (Millipore, Bedford, Mass.) or subjected to silver staining using standard techniques. Western blot analysis was performed with HRP-linked streptavidin (Invitrogen). Proteins were detected by enhanced chemiluminescence (Amersham). One specific biotinylated spot of ˜140 kDa was visible in the LAIR-ligand positive clone (FIG. S1B) that was absent in the wildtype Ba/F3 cells. The corresponding spot on the silver stained 2D-gel was subjected to mass spectrometry. In-gel proteolytic digestion of the silver-stained spot using trypsin (Roche) was performed essentially as described⁵. Samples were subjected to nanoflow liquid (LC) chromatography (Agilent 1100 series) and concentrated on a C18 precolumn (100 um ID, 2 cm). Peptides were separated on an analytical column (100 μM ID, 20 cm) at a flow rate of 200 nl/min with a 60 min linear acetonitrile gradient from 0 to 80%. The LC system was directly coupled to a QTOF Micro tandem mass spectrometer (Micromass Waters, UK). A survey scan was performed from 400-1200 amu s⁻¹ and precursor ions were sequenced in MS/MS mode at a threshold of 150 counts. Data were processed and subjected to database searches using Proteinlynx Global Server version 2.1 (Micromass, UK) or MASCOT software (Matrixscience) against SWISSPROT and the NCBI nonredundant database, with a 0.3 Da mass tolerance for both precursor ion and fragment ion. The identified peptide (DGFTGDLDYNK) was confirmed by manual interpretation of the spectra and proved identical to mouse collagen XVII.

Surface Plasmon Resonance Experiments.

Surface plasmon resonance (BIAcore) binding studies were performed using a BIAcore2000 system (BIAcore AB, Uppsala, Sweden). Approximately 2000-3000 response units (RU) of acid-soluble human collagen type I or III (Sigma) were immobilized on a CM5 biosensor chip using the amine coupling kit as instructed by the supplier. Immobilized triple-helical peptides composed of GCO(GPO)₁₀GCOG-NH₂ ((GPO)₁₀, also known as collagen-related peptide, CRP⁶) and GCP(GPP)₁₀GCPG-NH2 ((GPP)₁₀) were described previously⁶. Approximately 250 RU (GPP)₁₀ or (GPO)₁₀ peptide trimers were immobilized using a cysteine coupling kit according manufacturers instructions. Analysis was performed in buffer (125 mM NaCl, 2.5 mM CaCl2, 0.005% (v/v) Tween 20, and 25 mM Hepes, (pH 7.4)) at 25° C. at a flow rate of 20 μl/min for collagen I and III interaction-studies and 5 μl/min for the immobilized peptides. Binding of hLAIR-1-IgG to collagen I and III was specific, since nonspecific binding to an uncoated control channel was less than 2% compared to collagen-coated channels. In addition, a non-relevant IgG-fusion protein did not associate to the collagen-surface. hLAIR-1-IgG dimer concentration was calculated on the basis of theoretical mass of 85.2 kDa (corrected for removal of leader peptide). Increasing concentrations of hLAIR-1-IgG were injected and allowed to reach an equilibrium plateau for 10 minutes. The delay between injections was 13 min, during which time the biosensor chip was flushed with buffer. In case of peptide-binding studies, biosensor chips were regenerated by injection of 0.1 M H3PO4 (2 min, 5 μl/min).

Dissociation constants (Kd) and the number of binding sites expressed as the response at infinite hLAIR-1-IgG concentration (Bmax) were calculated as follows. First, the response at equilibrium (Req) was calculated for each association curve. Subsequently, Kd and Bmax were determined from the binding isotherms (Req plotted against hLAIR-1-IgG concentration) by fitting equation Req=Bmax*[hLAIR-1-IgG]/(Kd+[hLAIR-1-IgG]). The fit was calculated using computer program GraphPad Prism (GraphPad Prism version 3.00 for Windows; GraphPad Software, San Diego, Calif., USA). Bmax values were converted to number of hLAIR-1-IgG molecules interacting with a single collagen trimer using the theoretical mass of hLAIR-1-IgG (85.2 kDa) versus collagen I and III (416.7 and 415.7 kDa respectively).

The dissociation of hLAIR-1-IgG in the presence of buffer was followed for at least 13 hours and Koff-values were calculated using the Biaevaluation software version 3.0.1.

### Example 2

Crosslinking of LAIR-1 with Collagen Results in Inhibition of Primary T-Cell Function.

We investigated whether crosslinking of LAIR-1 with collagen on primary T cells could inhibit their function. To this end, PBMC were stimulated with αCD3 in the presence or absence of collagen and Elispot analysis was performed to determine the number of IFNγ producing T cells. Indeed, the production of IFNγ could be inhibited by the natural ligand of LAIR-1, collagen. Thus, cross-linking of LAIR-1 on primary T cells results in an inhibition of T-cell function. The results are shown in FIG. 6.

### Example 3

The entire triple-helical domain of human collagen II and III (termed the Collagen II and III Toolkits) were synthesized as a set of overlapping homotrimeric peptides and used these to screen for LAIR-1 binding sites by using LAIR-1 expressing cells in functional and adhesion assays. With these peptides we identified multiple binding sites for human LAIR-1 on both human collagens II and III, which functioned as potent agonists of LAIR-1 mediated inhibition of immune cell function.

Materials and Methods

Cells, Transfectant, and cDNA

Cell lines were obtained from the American Type Culture Collection (Manassas, Va.) and cultured using standard techniques. Cell lines that were used in this study are: human embryonic kidney 293T cells and human erythroleukemia K562 cells. Mouse 2B4 NFAT-GFP reporter T cell hybridoma cells were kindly provided by H. Arase and L. L. Lanier (UCSF, USA).

hLAIR1a, and hLAIR-1-CD3ζ cloned in the pMX puro retroviral vector were described previously (11). Cell lines stably expressing hLAIR-1a, or hLAIR-1-CD3ζ were generated as described previously (11).

Peptide Synthesis

The set of 57 overlapping synthetic peptides encompassing the entire triple-helical domain of human collagen III (Collagen III Toolkit) was described previously (5). The same approach was used to synthesize and verify 56 synthetic peptides encompassing the entire triple-helical domain of human collagen II (Toolkit collagen II). The primary sequences of the peptides of the Collagen II Toolkit and the Collagen III Toolkit are shown in Supplementary Table I and II respectively. Each peptide contains guest sequence (21): 27 amino acids of the human collagen II or III primary protein sequence, in which the C-terminal 9 amino acids form the first 9 guest amino acids of the next peptide. Thus, a 9-amino acid overlap is included between adjacent peptides (21). To ensure that the peptides are folded in a triple-helical conformation, each guest sequence was flanked between two (GPP)₅ host repeats, as described previously (5). The triple-helical peptides composed of GCO(GPO)₁₀GCOG-NH₂ ((GPO)₁₀, also known as collagen-related peptide, CRP (8)) and GCP(GPP)₁₀GCPG-NH₂ ((GPP)₁₀) were described previously (8). For GPO-requirement studies, we generated a set of GRPs in which the content of critical glycine-proline-hydroxyproline (GPO) triplets was varied in relation to the ‘inert’ scaffold sequence of GPP motifs (Toolbox peptides) (22). The sequences of these Toolbox peptides are shown in Supplementary table III.

Cell Adhesion Assay

96-well MAXIsorp (Nunc) flat-bottom plates were coated overnight at 4° C. with purified collagens I, III (Sigma), BSA, or synthetic trimeric peptides (100 μl/well, 10 μg/ml in PBS, 2 mM acetic acid). The cell adhesion assay was performed as described previously (11). K562 cells stably transduced with hLAIR-1a (11) were used in the assay.

Reporter Cell Assay

For detection of LAIR-1 ligands: 2B4 T cell hybridoma cells stably transduced with an NFAT-GFP reporter and hLAIR-1-CD3ζ were analyzed as described (11).

For detection of inhibition of mouse CD3 signaling: 2B4 T cell hybridoma cells stably transduced with an NFAT-GFP reporter and hLAIR-1a were generated. 1.25 μg/ml anti-mouse CD3 (PharMingen, San Diego, Calif.) was coated overnight at 4° C. in 96-well MAXIsorp flat-bottom plates (Nunc) together with an indicated amount of synthetic trimeric peptides in a total volume of 100 μl/well. The next day, plates were washed and 200 μl of 2.5×10⁵ reporter cells/ml in medium were added to each well, and plates were incubated at 37° C. for 22 hrs and then analyzed for GFP expression by flow cytometry on a FACSCalibur (BD Biosciences). Typically, ˜90 percent of the reporters expressed GFP upon CD3 stimulation. The percentage of inhibition of CD3 stimulation was calculated as follows: percentage of inhibition=100*[(% GFP⁺ cells in BSA coated well−% GFP⁺ cells in peptide coated well)/(% GFP⁺ cells in BSA coated well)].

Degranulation Assay

96-well MAXIsorp flat-bottom plates (Nunc) were coated overnight at 4° C. with BSA, or TNP conjugated to BSA (0.8 μg/ml) and indicated amounts of synthetic trimeric peptides (100 μl/well, 2 mM acetic acid in PBS). Meanwhile, untransfected or hLAIR-1a transfected RBL-2H3 (23) cells were sensitized with IgE anti-TNP mAbs at 4° C. for 30 minutes and subsequently washed for three times in medium (AIM-V; Gibco-BRL). After washings of the plates, 5.0×10⁵ cells in 70 μl medium were added to each well, and plates were incubated at 37° C. for 1 hour. Subsequently, cultures were transferred to PCR tubes, centrifuged and the supernatants were assayed for β-glucuronidase activity as described (23).

Results

Human LAIR-1 Binds Multiple Sites on Human Collagens II and III

To identify sites in human collagens that act as functional binding sites for the LAIR-1 molecules we used overlapping synthetic trimeric peptides encompassing the entire triple-helical domain of human collagens II and III. Fluorescently labeled K562 cells stably expressing human (h) LAIR-1 were monitored for their capacity to bind the immobilized collagen peptides in a cell adhesion assay. Human collagen II peptides 17, 30 and 56 and collagen III peptides 1, 30, 38, 44, 45, and 51 specifically interacted with hLAIR-1 expressed on K562 cells (FIGS. 7A and 8A, >10% adherence), but not with the parental K562 cells (data not shown). Additionally, K562 hLAIR-1 cells slightly adhered to several other peptides of collagen II and III (FIGS. 7A and 8A). To analyze whether the collagen peptides were able to induce functional cross-linking of hLAIR-1, we used 2B4 NFAT-GFP reporter cells (24) expressing a chimeric protein consisting of the extracellular domain of hLAIR-1 fused to the transmembrane and intracellular domain of CD3ζ (11). Receptor engagement of reporter cells expressing the hLAIR-1-CD3ζ chimera, via plate-bound collagen III, resulted in expression of GFP, indicating a functional triggering of the chimeric receptor (FIGS. 7B and 8B). The parental reporter cells which do not express the chimeric LAIR-1 receptor did not respond to collagen III (data not shown). Human collagen II peptides 1, 30, 42, 49 and 56 (FIG. 7B) and collagen III peptides 1, 5, 30, 32, 38, 44, 45, and 51 (FIG. 8B) induced profound GFP expression (>10% of cells turned GFP-positive) in hLAIR-1-CD3ζ expressing reporter cells. Furthermore, several peptides that induced a low percentage of GFP-expression (<10%) were apparent. None of the peptides induced GFP-expression in the parental 2B4 NFAT-GFP reporter cells (data not shown). Although the NFAT-GFP reporter cell assay identified the same peptides as ligands for hLAIR-1 as compared to the K562 cell adhesion assay (FIGS. 7A and B, and 8A and B), the NFAT-GFP reporter cell assay identified additional peptides as ligands for hLAIR-1 and is therefore a more sensitive ligand-sensing assay. The six most potent hLAIR-1 activators were in order of strength: III-30, II-56, II-30, III-38, II-42, and III-51.

Collagen II and III Synthetic Trimeric Peptides Identified by Toolkit are Potent Inhibitors of Immune Cell Function

LAIR-1 is a potent inhibitory immune receptor that is broadly expressed on immune cells (12). To assess whether the most effective peptides described above were also functional in cross-linking wild-type human LAIR-1 thereby inhibiting cellular immune responses, we performed two functional assays. First, we used 2B4 NFAT-GFP reporter cells (24) transfected with wild-type hLAIR-1. Cross-linking of the CD3 receptor on the surface of these reporter cells using plate-bound anti-mouse CD3 mAbs resulted in NFAT activation and GFP expression in approximately 90% of the cells (data not shown). Simultaneous cross-linking of hLAIR-1 via plate-bound CRP inhibited CD3 activation of hLAIR-1 transfected cells in a dose-dependent manner, but had little effect on the parental reporter cells (FIG. 9, top right panel). In contrast, the control (GPP)₁₀ peptides did not inhibit either cell-type (FIG. 9, top left panel). We next assessed the inhibitory potential of the four most potent peptides identified in FIGS. 7 and 8 (II-30, II-56, III-38, and III-30). As expected, all four peptides were capable of efficiently inhibiting the CD3-induced activation of the hLAIR-1 transfected T cell hybridoma cells (FIG. 9, middle panels). At low peptide concentrations (1.1 μg/ml), peptides II-56 and III-30 maintained their full inhibitory capacity, whereas the II-30 and III-38 peptides only inhibited ˜60% of the T cells and the inhibitory potential of CRP was completely lost (FIG. 9). Two peptides from the collagen II peptide toolkit that did not respond in the previous assays also did not induce inhibition via hLAIR-1 (peptides II-20 and II-47, FIG. 9, bottom panels). None of the peptides were efficient in inhibiting the parental reporter T cell hybridoma, although the most potent peptides (II-56 and III-30) did induce moderate inhibition (˜30%) of these cells at high peptide concentrations (FIG. 9). This is probably because the 2B4 NFAT-reporter cells express low amounts of endogenous mLAIR-1 which account for the observed inhibition by the peptides (data not shown).

In a second functional assay to test the inhibitory potential of the selected collagen peptides we used hLAIR-1 transfected RBL-2H3 cells, which express endogenous IgE receptor FcεRI (23). We previously showed that collagens I, III, and CRP were capable of specifically inhibiting the FcεRI-induced degranulation of these cells (11). As expected, peptides II-56 and III-30 were also potent inhibitors of the degranulation response (FIG. 10), whereas control peptides (GPP)₁₀ and II-47 were not. Again, peptides II-56 and III-30 proved more efficient functional ligands than CRP. Thus, the identified synthetic trimeric collagen peptides are potent inducers of LAIR-1-mediated inhibition of immune responses.

GPO-Content in Peptides Correlates with Binding Potential to LAIR-1

Using BIAcore, we have shown that hLAIR-1 interacts with triple-helical peptides composed of 10 repeated GPO triplets (CRP), but not with corresponding peptides consisting of 10 repeated GPP triplets ((GPP)₁₀) (11). To analyze whether the occurrence of GPO triplets in the collagen II and III Toolkit peptides correlates with LAIR-1 binding, we counted GPO triplets in peptides able to bind human LAIR-1. As expected, peptides with increased GPO-content were more likely to act as LAIR-1 ligands (FIG. 11A). Additionally, the 4 most potent hLAIR-1 activators II-30, II-56, III-30 and III-38 contained 2, 4, 3, and 3 GPO triplets respectively, whereas the average GPO content in the collagen II and III peptides is only ˜0.91 per peptide. To investigate whether an increased content of GPO triplets in our synthetic peptides also resulted in more potent LAIR-1 ligands, we used peptides containing 0, 1, 2, 4, 6, or 10 adjacent GPOs triplets in a GPP backbone (Toolbox peptides) (22,25). As expected, we observed a stepwise increase in both the inhibition of the degranulation response of RBL-2H3 cells (FIG. 11B) and the inhibition of CD3 stimulated 2B4 NFAT-GFP reporter cells (FIG. 11C) upon increased GPO content of the peptides. Nevertheless, although the Toolbox peptides could functionally interact with hLAIR-1, they were much less efficient in inhibiting immune responses compared with the newly identified collagen peptides described above (II-30, II-56, III-30 and III-38). For example, whereas peptide II-56 retained full inhibitory potential via LAIR-1 at even low peptide concentrations (1.1 μg/ml), the inhibitory potential of the most potent Toolbox peptide GPO₁₀ (CRP) was completely absent (FIG. 9).

Intriguingly, although the (GPP)₁₀ control peptides showed no/hardly binding to LAIR-1, two GPO-deficient peptides from the collagen III Toolkit were potent LAIR-1 binders and activators peptides 51 and 32. Although these peptides did contain hydroxyproline residues, the content of these residues was not elevated as compared to GPO-lacking peptides that did not bind human LAIR-1 (data not shown).

Supplemental TABLE I
Peptide sequences of the collagen II Toolkit.

| # | Sequences |
|---|---|
| 1 | GPC(GPP)₅-GPMGPMGPRGPOGPAGAOGPQGFQGNO- |
| | (GPP)₅GPC-NH₂ |
| 2 | GPC(GPP)₅-GPQGFQGNOGEOGEOGVSGPMGPRGPO- |
| | (GPP)₅GPC-NH₂ |
| 3 | GPC(GPP)₅-GPMGPRGPOGPOGKOGDDGEAGKOGKA- |
| | (GPP)₅GPC-NH₂ |
| 4 | GPC(GPP)₅-GEAGKOGKAGERGPOGPQGARGFOGTO- |
| | (GPP)₅GPC-NH₂ |
| 5 | GPC(GPP)₅-GARGFOGTOGLOGVKGHRGYOGLDGAK- |
| | (GPP)₅GPC-NH₂ |
| 6 | GPC(GPP)₅-GYOGLDGAKGEAGAOGVKGESGSOGEN- |
| | (GPP)₅GPC-NH₂ |
| 7 | GPC(GPP)₅-GESGSOGENGSOGPMGPRGLOGERGRT- |
| | (GPP)₅GPC-NH₂ |
| 8 | GPC(GPP)₅-GLOGERGRTGPAGAAGARGNDGQOGPA- |
| | (GPP)₅GPC-NH₂ |
| 9 | GPC(GPP)₅-GNDGQOGPAGPOGPVGPAGGOGFOGAO- |
| | (GPP)₅GPC-NH₂ |
| 10 | GPC(GPP)₅-GGOGFOGAOGAKGEAGPTGARGPEGAQ- |
| | (GPP)₅GPC-NH₂ |
| 11 | GPC(GPP)₅-GARGPEGAQGPRGEOGTOGSOGPAGAS- |
| | (GPP)₅GPC-NH₂ |
| 12 | GPC(GPP)₅-GSOGPAGASGNOGTDGIOGAKGSAGAO- |
| | (GPP)₅GPC-NH₂ |
| 13 | GPC(GPP)₅-GAKGSAGAOGIAGAOGFOGPRGPOGPQ- |
| | (GPP)₅GPC-NH₂ |
| 14 | GPC(GPP)₅-GPRGPOGPQGATGPLGPKGQTGEOGIA- |
| | (GPP)₅GPC-NH₂ |
| 15 | GPC(GPP)₅-GQTGEOGIAGFKGEQGPKGEOGPAGPQ- |
| | (GPP)₅GPC-NH₂ |
| 16 | GPC(GPP)₅-GEOGPAGPQGAOGPAGEEGKRGARGEO- |
| | (GPP)₅GPC-NH₂ |
| 17 | GPC(GPP)₅-GKRGARGEOGGVGPIGPOGERGAOGNR- |
| | (GPP)₅GPC-NH₂ |
| 18 | GPC(GPP)₅-GERGAOGNRGFOGQDGLAGPKGAOGER- |
| | (GPP)₅GPC-NH₂ |
| 19 | GPC(GPP)₅-GPKGAOGERGPSGLAGPKGANGDOGRO- |
| | (GPP)₅GPC-NH₂ |
| 20 | GPC(GPP)₅-GANGDOGROGEOGLOGARGLTGROGDA- |
| | (GPP)₅GPC-NH₂ |
| 21 | GPC(GPP)₅-GLTGROGDAGPQGKVGPSGAOGEDGRO- |
| | (GPP)₅GPC-NH₂ |
| 22 | GPC(GPP)₅-GAOGEDGROGPOGPQGARGQOGVMGFO- |
| | (GPP)₅GPC-NH₂ |
| 23 | GPC(GPP)₅-GQOGVMGFOGPKGANGEOGKAGEKGLO- |
| | (GPP)₅GPC-NH₂ |
| 24 | GPC(GPP)₅-GKAGEKGLOGAOGLRGLOGKDGETGAA- |
| | (GPP)₅GPC-NH₂ |
| 25 | GPC(GPP)₅-GKDGETGAAGPOGPAGPAGERGEQGAO- |
| | (GPP)₅GPC-NH₂ |
| 26 | GPC(GPP)₅-GERGEQGAOGPSGFQGLOGPOGPOGEG- |
| | (GPP)₅GPC-NH₂ |
| 27 | GPC(GPP)₅-GPOGPOGEGGKOGDQGVOGEAGAOGLV- |
| | (GPP)₅GPC-NH₂ |
| 28 | GPC(GPP)₅-GEAGAOGLVGPRGERGFOGERGSOGAQ- |
| | (GPP)₅GPC-NH₂ |
| 29 | GPC(GPP)₅-GERGSOGAQGLQGPRGLOGTOGTDGPK- |
| | (GPP)₅GPC-NH₂ |
| 30 | GPC(GPP)₅-GTOGTDGPKGASGPAGPOGAQGPOGLQ- |
| | (GPP)₅GPC-NH₂ |
| 31 | GPC(GPP)₅-GAQGPOGLQGMOGERGAAGIAGPKGDR- |
| | (GPP)₅GPC-NH₂ |
| 32 | GPC(GPP)₅-GIAGPKGDRGDVGEKGPEGAOGKDGGR- |
| | (GPP)₅GPC-NH₂ |
| 33 | GPC(GPP)₅-GAOGKDGGRGLTGPIGPOGPAGANGEK- |
| | (GPP)₅GPC-NH₂ |
| 34 | GPC(GPP)₅-GPAGANGEKGEVGPOGPAGSAGARGAO- |
| | (GPP)₅GPC-NH₂ |
| 35 | GPC(GPP)₅-GSAGARGAOGERGETGPOGPAGFAGPO- |
| | (GPP)₅GPC-NH₂ |
| 36 | GPC(GPP)₅-GPAGFAGPOGADGQOGAKGEQGEAGQK- |
| | (GPP)₅GPC-NH₂ |
| 37 | GPC(GPP)₅-GEQGEAGQKGDAGAOGPQGPSGAOGPQ- |
| | (GPP)₅GPC-NH₂ |
| 38 | GPC(GPP)₅-GPSGAOGPQGPTGVTGPKGARGAQGPO- |
| | (GPP)₅GPC-NH₂ |
| 39 | GPC(GPP)₅-GARGAQGPOGATGFOGAAGRVGPOGSN- |
| | (GPP)₅GPC-NH₂ |
| 40 | GPC(GPP)₅-GRVGPOGSNGNOGPOGPOGPSGKDGPK- |
| | (GPP)₅GPC-NH₂ |
| 41 | GPC(GPP)₅-GPSGKDGPKGARGDSGPOGRAGEOGLQ- |
| | (GPP)₅GPC-NH₂ |
| 42 | GPC(GPP)₅-GRAGEOGLQGPAGPOGEKGEOGDDGPS- |
| | (GPP)₅GPC-NH₂ |
| 43 | GPC(GPP)₅-GEOGDDGPSGAEGPOGPQGLAGQRGIV- |
| | (GPP)₅GPC-NH₂ |
| 44 | GPC(GPP)₅-GLAGQRGIVGLOGQRGERGFOGLOGPS- |
| | (GPP)₅GPC-NH₂ |
| 45 | GPC(GPP)₅-GFOGLOGPSGEOGKQGAOGASGDRGPO- |
| | (GPP)₅GPC-NH₂ |
| 46 | GPC(GPP)₅-GASGDRGPOGPVGPOGLTGPAGEOGRE- |
| | (GPP)₅GPC-NH₂ |
| 47 | GPC(GPP)₅-GPAGEOGREGSOGADGPOGRDGAAGVK- |
| | (GPP)₅GPC-NH₂ |
| 48 | GPC(GPP)₅-GRDGAAGVKGDRGETGAVGAOGAOGPO- |
| | (GPP)₅GPC-NH₂ |
| 49 | GPC(GPP)₅-GAOGAOGPOGSOGPAGPTGKQGDRGEA- |
| | (GPP)₅GPC-NH₂ |
| 50 | GPC(GPP)₅-GKQGDRGEAGAQGPMGPSGPAGARGIQ- |
| | (GPP)₅GPC-NH₂ |
| 51 | GPC(GPP)₅-GPAGARGIQGPQGPRGDKGEAGEOGER- |
| | (GPP)₅GPC-NH₂ |
| 52 | GPC(GPP)₅-GEAGEOGERGLKGHRGFTGLQGLOGPO- |
| | (GPP)₅GPC-NH₂ |
| 53 | GPC(GPP)₅-GLQGLOGPOGPSGDQGASGPAGPSGPR- |
| | (GPP)₅GPC-NH₂ |
| 54 | GPC(GPP)₅-GPAGPSGPRGPOGPVGPSGKDGANGIO- |
| | (GPP)₅GPC-NH₂ |
| 55 | GPC(GPP)₅-GKDGANGIOGPIGPOGPRGRSGETGPA- |
| | (GPP)₅GPC-NH₂ |
| 56 | GPC(GPP)₅-GPRGRSGETGPAGPOGNOGPOGPOGPO- |
| | (GPP)₅GPC-NH₂ |

Synthetic Methods A and B were described previously (5). GPO residues are shown in boldface.

Supplemental TABLE II
Peptide sequences of the collagen III Toolkit.

| # | Sequences |
|---|---|
| 1 | GPC(GPP)₅-GLAGYOGPAGPOGPOGPOGTSGHOGSO- |
| | (GPP)₅GPC-NH₂ |
| 2 | GPC(GPP)₅-GTSGHOGSOGSOGYQGPOGEOGQAGPS- |
| | (GPP)₅GPC-NH₂ |
| 3 | GPC(GPP)₅-GEOGQAGPSGPOGPOGAIGPSGPAGKD- |
| | (GPP)₅GPC-NH₂ |
| 4 | GPC(GPP)₅-GPSGPAGKDGESGROGROGERGLOGPO- |
| | (GPP)₅GPC-NH₂ |
| 5 | GPC(GPP)₅-GERGLOGPOGIKGPAGIOGFOGMKGHR- |
| | (GPP)₅GPC-NH₂ |
| 6 | GPC(GPP)₅-GFOGMKGHRGFDGRNGEKGETGAOGLK- |
| | (GPP)₅GPC-NH₂ |
| 7 | GPC(GPP)₅-GETGAOGLKGENGLOGENGAOGPMGPR- |
| | (GPP)₅GPC-NH₂ |
| 8 | GPC(GPP)₅-GAOGPMGPRGAOGERGROGLOGAAGAR- |
| | (GPP)₅GPC-NH₂ |
| 9 | GPC(GPP)₅-GLOGAAGARGNDGARGSDGQOGPOGPO- |
| | (GPP)₅GPC-NH₂ |
| 10 | GPC(GPP)₅-GQOGPOGPOGTAGFOGSOGAKGEVGPA- |
| | (GPP)₅GPC-NH₂ |
| 11 | GPC(GPP)₅-GAKGEVGPAGSOGSNGAOGQRGEOGPQ- |
| | (GPP)₅GPC-NH₂ |
| 12 | GPC(GPP)₅-GQRGEOGPQGHAGAQGPOGPOGINGSO- |
| | (GPP)₅GPC-NH₂ |
| 13 | GPC(GPP)₅-GPOGINGSOGGKGEMGPAGIOGAOGLM- |
| | (GPP)₅GPC-NH₂ |
| 14 | GPC(GPP)₅-GIOGAOGLMGARGPOGPAGANGAOGLR- |
| | (GPP)₅GPC-NH₂ |
| 15 | GPC(GPP)₅-GANGAOGLRGGAGEOGKNGAKGEOGPR- |
| | (GPP)₅GPC-NH₂ |
| 16 | GPC(GPP)₅-GAKGEOGPRGERGEAGIOGVOGAKGED- |
| | (GPP)₅GPC-NH₂ |
| 17 | GPC(GPP)₅-GVOGAKGEDGKDGSOGEOGANGLOGAA- |
| | (GPP)₅GPC-NH₂ |
| 18 | GPC(GPP)₅-GANGLOGAAGERGAOGFRGPAGPNGIO- |
| | (GPP)₅GPC-NH₂ |
| 19 | GPC(GPP)₅-GPAGPNGIOGEKGPAGERGAOGPAGPR- |
| | (GPP)₅GPC-NH₂ |
| 20 | GPC(GPP)₅-GAOGPAGPRGAAGEOGRDGVOGGOGMR- |
| | (GPP)₅GPC-NH₂ |
| 21 | GPC(GPP)₅-GVOGGOGMRGMOGSOGGOGSDGKOGPO- |
| | (GPP)₅GPC-NH₂ |
| 22 | GPC(GPP)₅-GSDGKOGPOGSQGESGROGPOGPSGPR- |
| | (GPP)₅GPC-NH₂ |
| 23 | GPC(GPP)₅-GPOGPSGPRGQOGVMGFOGPKGNDGAO- |
| | (GPP)₅GPC-NH₂ |
| 24 | GPC(GPP)₅-GPKGNDGAOGKNGERGGOGGOGPQGPO- |
| | (GPP)₅GPC-NH₂ |
| 25 | GPC(GPP)₅-GGOGPQGPOGKNGETGPQGPOGPTGPG- |
| | (GPP)₅GPC-NH₂ |
| 26 | GPC(GPP)₅-GPOGPTGPGGDKGDTGPOGPQGLQGLO- |
| | (GPP)₅GPC-NH₂ |
| 27 | GPC(GPP)₅-GPQGLQGLOGTGGPOGENGKOGEOGPK- |
| | (GPP)₅GPC-NH₂ |
| 28 | GPC(GPP)₅-GKOGEOGPKGDAGAOGAOGGKGDAGAO- |
| | (GPP)₅GPC-NH₂ |
| 29 | GPC(GPP)₅-GGKGDAGAOGERGPOGLAGAOGLRGGA- |
| | (GPP)₅GPC-NH₂ |
| 30 | GPC(GPP)₅-GAOGLRGGAGPOGPEGGKGAAGPOGPO- |
| | (GPP)₅GPC-NH₂ |
| 31 | GPC(GPP)₅-GAAGPOGPOGAAGTOGLQGMOGERGGL- |
| | (GPP)₅GPC-NH₂ |
| 32 | GPC(GPP)₅-GMOGERGGLGSOGPKGDKGEOGGOGAD- |
| | (GPP)₅GPC-NH₂ |
| 33 | GPC(GPP)₅-GEOGGOGADGVOGKDGPRGPTGPIGPO- |
| | (GPP)₅GPC-NH₂ |
| 34 | GPC(GPP)₅-GPTGPIGPOGPAGQOGDKGEGGAOGLO- |
| | (GPP)₅GPC-NH₂ |
| 35 | GPC(GPP)₅-GEGGAOGLOGIAGPRGSOGERGETGPO- |
| | (GPP)₅GPC-NH₂ |
| 36 | GPC(GPP)₅-GERGETGPOGPAGFOGAOGQNGEOGGK- |
| | (GPP)₅GPC-NH₂ |
| 37 | GPC(GPP)₅-GQNGEOGGKGERGAOGEKGEGGPOGVA- |
| | (GPP)₅GPC-NH₂ |
| 38 | GPC(GPP)₅-GEGGPOGVAGPOGGSGPAGPOGPQGVK- |
| | (GPP)₅GPC-NH₂ |
| 39 | GPC(GPP)₅-GPOGPQGVKGERGSOGGOGAAGFOGAR- |
| | (GPP)₅GPC-NH₂ |
| 40 | GPC(GPP)₅-GAAGFOGARGLOGPOGSNGNOGPOGPS- |
| | (GPP)₅GPC-NH₂ |
| 41 | GPC(GPP)₅-GNOGPOGPSGSOGKDGPOGPAGNTGAO- |
| | (GPP)₅GPC-NH₂ |
| 42 | GPC(GPP)₅-GPAGNTGAOGSOGVSGPKGDAGQOGEK- |
| | (GPP)₅GPC-NH₂ |
| 43 | GPC(GPP)₅-GDAGQOGEKGSOGAQGPOGAOGPLGIA- |
| | (GPP)₅GPC-NH₂ |
| 44 | GPC(GPP)₅-GAOGPLGIAGITGARGLAGPOGMOGPR- |
| | (GPP)₅GPC-NH₂ |
| 45 | GPC(GPP)₅-GPOGMOGPRGSOGPQGVKGESGKOGAN- |
| | (GPP)₅GPC-NH₂ |
| 46 | GPC(GPP)₅-GESGKOGANGLSGERGPOGPQGLOGLA- |
| | (GPP)₅GPC-NH₂ |
| 47 | GPC(GPP)₅-GPQGLOGLAGTAGEOGRDGNOGSDGLO- |
| | (GPP)₅GPC-NH₂ |
| 48 | GPC(GPP)₅-GNOGSDGLOGRDGSOGGKGDRGENGSO- |
| | (GPP)₅GPC-NH₂ |
| 49 | GPC(GPP)₅-GDRGENGSOGAOGAOGHOGPOGPVGPA- |
| | (GPP)₅GPC-NH₂ |
| 50 | GPC(GPP)₅-GPOGPVGPAGKSGDRGESGPAGPAGAO- |
| | (GPP)₅GPC-NH₂ |
| 51 | GPC(GPP)₅-GPAGPAGAOGPAGSRGAOGPQGPRGDK- |
| | (GPP)₅GPC-NH₂ |
| 52 | GPC(GPP)₅-GPQGPRGDKGETGERGAAGIKGHRGFO- |
| | (GPP)₅GPO-NH₂ |
| 53 | GPC(GPP)₅-GIKGHRGFOGNOGAOGSOGPAGQQGAI- |
| | (GPP)₅GPC-NH₂ |
| 54 | GPCCGPP)₅-GPAGQQGAIGSOGPAGPRGPVGPSGPO- |
| | (GPP)₅GPC-NH₂ |
| 55 | GPC(GPP)₅-GPVGPSGPOGKDGTSGHOGPIGPOGPR- |
| | (GPP)₅GPC-NH₂ |
| 56 | GPC(GPP)₅-GPIGPOGPRGNRGERGSEGSOGHOGQO- |
| | (GPP)₅GPC-NH₂ |
| 57 | GPC(GPP)₅-GERGSEGSOGHOGQOGPOGPOGAOGPC- |
| | (GPP)₅GPC-NH₂ |

Synthetic Methods A and B were described previously (5). GPO residues are shown in boldface.

Supplemental TABLE III
Peptide sequences of Toolbox peptide 30
variants

| # | Sequences |
|---|---|
| (GPP)10 | GPC-GPPGPPGPPGPPGPPGPPGPPGPPGPPGPP-GPC-NH₂ |
| CRP | GPC-GPOGPOGPOGPOGPOGPOGPOGPOGPOGPO-GPC-NH₂ |
| (GPO)1 | GPC-GPPGPPGPPGPPGPPGPOGPPGPPGPPGPP-GPC-NH₂ |
| (GPO)2 | GPC-GPPGPPGPPGPPGPOGPOGPPGPPGPPGPP-GPC-NH₂ |
| (GPO)4 | GPC-GPPGPPGPPGPOGPOGPOGPOGPPGPPGPP-GPC-NH₂ |
| (GPO)6 | GPC-GPPGPPGPOGPOGPOGPOGPOGPOGPPGPP-GPC-NH₂ |

Synthetic Methods A and B were described previously (5). GPO residues are shown in boldface.

### REFERENCE LIST OF EXAMPLE 3

1 Myllyharju, J. and Kivirikko, K. I. 2004. Collagens, modifying enzymes and their mutations in humans, flies and worms. Trends Genet. 20:33-43.
2 Ricard-Blum, S. and Ruggiero, F. 2005. The collagen superfamily: from the extracellular matrix to the cell membrane. Pathol. Biol. (Paris) 53:430-442.
3 Gelse, K., Poschl, E., and Aigner, T. 2003. Collagens—structure, function, and biosynthesis. Adv. Drug Deliv. Rev. 55:1531-1546.
4 Farndale, R. W., Sixma, J. J., Barnes, M. J., and de Groot, P. G. 2004. The role of collagen in thrombosis and hemostasis. J. Thromb. Haemost. 2:561-573.
5 Raynal, N., Hamaia, S. W., Siljander, P. R., Maddox, B., Peachey, A. R., Fernandez, R., Foley, L. J., Slatter, D. A., Jarvis, G. E., and Farndale, R. W. 2006. Use of synthetic peptides to locate novel integrin alpha2beta1-binding motifs in human collagen III. J. Biol. Chem. Feb. 17, 2006;281. (7.):3821.-31. Epub. Dec. 2, 2005. 281:3821-3831.
6 Lisman, T., Raynal, N., Groeneveld, D., Maddox, B., Peachey, A. R., Huizinga, E. G., de Groot, P. G., and Farndale, R. W. 2006. A single high-affinity binding site for von Willebrand Factor in collagen III, identified using synthetic triple-helical peptides. Blood. Aug. 15, 2006;
7 Kehrel, B., Wierwille, S., Clemetson, K. J., Anders, O., Steiner, M., Knight, C. G., Farndale, R. W., Okuma, M., and Barnes, M. J. 1998. Glycoprotein VI is a major collagen receptor for platelet activation: it recognizes the platelet-activating quaternary structure of collagen, whereas CD36, glycoprotein IIb/IIIa, and von Willebrand factor do not. Blood 91:491-499.
8 Knight, C. G., Morton, L. F., Onley, D. J., Peachey, A. R., Ichinohe, T., Okuma, M., Farndale, R. W., and Barnes, M. J. 1999. Collagen-platelet interaction: Gly-Pro-Hyp is uniquely specific for platelet Gp VI and mediates platelet activation by collagen. Cardiovasc. Res. 41:450-457.
9 Morton, L. F., Hargreaves, P. G., Farndale, R. W., Young, R. D., and Barnes, M. J. 1995. Integrin alpha 2 beta 1-independent activation of platelets by simple collagen-like peptides: collagen tertiary (triple-helical) and quaternary (polymeric) structures are sufficient alone for alpha 2 beta 1-independent platelet reactivity. Biochem. J. 306 (Pt 2):337-344.
10 Moroi, M. and Jung, S. M. 2004. Platelet glycoprotein VI: its structure and function. Thromb. Res. 114:221-233.
11 Lebbink, R. J., de Ruiter, T., Adelmeijer, J., Brenkman, A. B., van Helvoort, J. M., Koch, M., Farndale, R. W., Lisman, T., Sonnenberg, A., Lenting, P. J., and Meyaard, L. 2006. Collagens are functional, high affinity ligands for the inhibitory immune receptor LAIR-1. J. Exp. Med. 203:1419-1425.
12 Meyaard, L., Adema, G. J., Chang, C., Woollatt, E., Sutherland, G. R., Lanier, L. L., and Phillips, J. H. 1997. LAIR-1, a novel inhibitory receptor expressed on human mononuclear leukocytes. Immunity. 7:283-290.
13 Poggi, A., Pella, N., Morelli, L., Spada, F., Revello, V., Sivori, S., Augugliaro, R., Moretta, L., and Moretta, A. 1995. p 40, a novel surface molecule involved in the regulation of the non-major histocompatibility complex-restricted cytolytic activity in humans. Eur. J. Immunol. 25:369-376.
14 Meyaard, L., Hurenkamp, J., Clevers, H., Lanier, L. L., and Phillips, J. H. 1999. Leukocyte-associated Ig-like receptor-1 functions as an inhibitory receptor on cytotoxic T cells. J. Immunol. 162:5800-5804.
15 Saverino, D., Fabbi, M., Merlo, A., Ravera, G., Grossi, C. E., and Ciccone, E. 2002. Surface density expression of the leukocyte-associated Ig-like receptor-1 is directly related to inhibition of human T-cell functions. Hum. Immunol. 2002. July;63. (7.):534.-46.63:534-546.
16 Maasho, K., Masilamani, M., Valas, R., Basu, S., Coligan, J. E., and Borrego, F. 2005. The inhibitory leukocyte-associated Ig-like receptor-1 (LAIR-1) is expressed at high levels by human naive T cells and inhibits TCR mediated activation. Mol. Immunol. 42:1521-1530.
17 van der Vuurst de Vries A R, Clevers, H., Logtenberg, T., and Meyaard, L. 1999. Leukocyte-associated immunoglobulin-like receptor-1 (LAIR-1) is differentially expressed during human B cell differentiation and inhibits B cell receptor-mediated signaling. Eur. J. Immunol. 29:3160-3167.
18 Poggi, A., Tomasello, E., Ferrero, E., Zocchi, M. R., and Moretta, L. 1998. p40/LAIR-1 regulates the differentiation of peripheral blood precursors to dendritic cells induced by granulocyte-monocyte colony-stimulating factor. Eur. J. Immunol. 28:2086-2091.
19 Lebbink, R. J., Kaptijn, G. J., de Ruiter, T., Jansen, C. A., Lenting, P. J., and Meyaard, L. 2006. Mouse Leukocyte-associated Ig-like receptor-1 (LAIR-1) functions as an inhibitory collagen-binding receptor on immune cells. In.
20 Ravetch, J. V. and Lanier, L. L. 2000. Immune inhibitory receptors. Science 290:84-89.
21 Shah, N. K., Ramshaw, J. A., Kirkpatrick, A., Shah, C., and Brodsky, B. 1996. A host-guest set of triple-helical peptides: stability of Gly-X-Y triplets containing common nonpolar residues. Biochemistry. 35:10262-10268.
22 O'Connor, M. N., Smethurst, P. A., Davies, L. W., Joutsi-Korhonen, L., Onley, D. J., Herr, A. B., Farndale, R. W., and Ouwehand, W. H. 2006. Selective blockade of glycoprotein VI clustering on collagen helices. J. Biol. Chem. Sep. 6, 2006;
23 Verbrugge, A., de Ruiter, T., Clevers, H., and Meyaard, L. 2003. Differential contribution of the Immunoreceptor Tyrosine-based Inhibitory Motifs (ITIMs) of human Leukocyte Associated Ig-like Receptor (LAIR)-1 to inhibitory function and phosphatase recruitment. Int. Immunol. 15:1349-1358.
24 Voehringer, D., Rosen, D. B., Lanier, L. L., and Locksley, R. M. 2004. CD200 receptor family members represent novel DAP12-associated activating receptors on basophils and mast cells. J. Biol. Chem. 279:54117-54123.
25 Farndale, R. W., Siljander, P. R., Onley, D. J., Sundaresan, P., Knight, C. G., and Barnes, M. J. 2003. Collagen-platelet interactions: recognition and signalling. Biochem. Soc. Symp. 2003.;(70.):81.-94.81-94.
26 Miura, Y., Takahashi, T., Jung, S. M., and Moroi, M. 2002. Analysis of the interaction of platelet collagen receptor glycoprotein VI (GPVI) with collagen. A dimeric form of GPVI, but not the monomeric form, shows affinity to fibrous collagen. J. Biol. Chem. 277:46197-46204.
27 Horii, K., Kahn, M. L., and Herr, A. B. 2006. Structural basis for platelet collagen responses by the immune-type receptor glycoprotein VI. Blood.
28 Lebbink, R. J., de Ruiter, T., Verbrugge, A., Bril, W. S., and Meyaard, L. 2004. The mouse homologue of the leukocyte-associated Ig-like receptor-1 is an inhibitory receptor that recruits Src homology region 2-containing protein tyrosine phosphatase (SHP)-2, but not SHP-1. J. Immunol. 172:5535-5543.
29 Smethurst, P. A., Joutsi-Korhonen, L., O'Connor, M. N., Wilson, E., Jennings, N. S., Garner, S. F., Zhang, Y., Knight, C. G., Dafforn, T. R., Buckle, A., IJsseldijk, M. J., de Groot, P. G., Watkins, N. A., Farndale, R. W., and Ouwehand, W. H. 2004. Identification of the primary collagen-binding surface on human glycoprotein VI by site-directed mutagenesis and by a blocking phage antibody. Blood 103:903-911.
30 Smethurst, P. A., Onley, D. J., Jarvis, G. E., O'Connor, M. N., Knight, C. G., Herr, A. B., Ouwehand, W., and Farndale, R. W. 2006. The platelet-collagen interaction: the smallest effective motif for Glycoprotein VI-mediated platelet adhesion and activation contains two Glycine-Proline-Hydroxyproline triplets. In.

### Example 4

Leukocyte-Associated Immunoglobulin-like Receptor (LAIR)-2 is a Soluble Competitor of the Collagen/LAIR-1 Inhibitory Immune Interaction

In this example we explored whether LAIR-2 is secreted and whether it functions as a soluble competitor for the interaction between collagens and hLAIR-1.

Results

LAIR-2 is Expressed by Immune Cells

To assess the cellular distribution of LAIR-2, several human cell lines were analyzed by RT PCR specific for LAIR-2 expression. Transcripts were detected in cells of hematopoietic origin (FIG. 12A), corresponding with the expression pattern of hLAIR-1(4, 9-11). LAIR-2 was detected in primary cells (PBMCs), monocytic and T cell lines, but not in non-hematopoietic cell lines. Besides the previously described LAIR-2a and LAIR-2b(13) isoforms, an additional product was evident after RT-PCR (FIG. 12A). Cloning, sequencing and subsequent sequence alignment of this product demonstrated it was a third splice variant, which we designated LAIR-2c. LAIR-2c lacks 93 bp at the 3′end of exon 3, encoding a putative protein with a 31 amino acid deletion as compared to LAIR-2a. The 31 aa deletion encompasses a region in the hLAIR-1 protein where one of the conserved cysteines involved in intradomain disulfide bond formation is located(13). Thus this splice variant does not encode an intact Ig-like domain. The DNA sequences of LAIR-2c was deposited in the GenBank™ database under accession number EF174570.

LAIR-2 is a Secreted Protein

The presence of a signal sequence and the lack of a transmembrane and cytoplasmic region suggests that LAIR-2 is a secreted protein(9). In order to detect expression of the LAIR-2 protein, anti-LAIR-2 mAbs were generated which specifically stained 293T cells transfected with LAIR-2, but not untransfected cells (FIG. 12B). The antibody did not cross-react with human LAIR-1 (data not shown). As expected, LAIR-2 was present in the supernatant of 293T cells transiently transfected with LAIR-2, but was not detected in the supernatant of untransfected cells (FIG. 12C). The protein was present as a smear on non-reducing Western blot, ranging from an apparent molecular mass of ˜70 to ˜30 kDa. The protein could not be detected under reducing conditions. Monomeric LAIR-2a has a predicted molecular mass of ˜16.3 kDa and the protein contains 10 putative O-linked glycosylation sites but no N-linked glycosylation sites, whereas LAIR-2b has a predicted molecular mass of ˜14.7 kDa containing 4 putative O-linked glycosylation sites (data not shown). Hence, the higher molecular weight as seen on Western blot is apparently caused by various states of multimerization and O-linked glycosylation of the LAIR-2 protein. We conclude that the LAIR-2 gene encodes a secreted protein.

LAIR-2 is a High Affinity Collagen-Receptor

Recently, we identified collagens as high affinity ligands for the LAIR-1 molecules(15). The collagen super family comprises 28 trimeric molecules each composed of three polypeptide a chains, which contain the sequence repeat (Gly-X-Y)ₙ, X being frequently proline (P) and, after post-translational modification, Y being hydroxyproline (O)(21). Besides hLAIR-1, LAIR-2-IgG fusion proteins interacted with transmembrane collagens XIII, XVII and XXIII(15), showing that all LAIR-molecules bind the same collagen molecules as ligand. In order to determine whether LAIR-2 has the potential to serve as a competitor for hLAIR-1 binding by blocking binding sites on human collagens, we measured binding and affinity of LAIR-2-IgG fusion proteins to collagen by surface plasmon resonance (BIAcore). As expected, LAIR-2 fusion proteins bind with high affinity of 34.6 nM (±3.5) and 41.4 nM (±3.7) to collagen I and III, respectively (FIG. 13A). Each collagen molecule has ˜10 binding sites for LAIR-2 (FIG. 13A). The dissociation of LAIR-2 is biphasic and characterized by a rapid initial phase and a slower secondary phase (FIG. 13B).

The GPO triplet is almost exclusively present in collagenous molecules, where it comprises about 10% of the mature collagen sequence, and allows the formation of a triple helix(21). Like hLAIR-1, LAIR-2 binds common collagen motifs in a hydroxyproline-dependent manner, since it interacts with trimeric peptides containing 10 repeated GPO triplets ((GPO)₁₀(22)), but not with the control (GPP)₁₀ trimeric peptide (FIG. 13C).

LAIR-2 Prevents Binding of hLAIR-1 to its Ligand

Since LAIR-2 is a secreted protein and interacts with the same collagen molecules as hLAIR-1, LAIR-2 functions as a competitor of the hLAIR-1/collagen interaction by binding the same sites on human collagen molecules. To investigate this, we performed LAIR blocking experiments using human HT29 colon carcinoma cells expressing endogenous hLAIR-1 ligand(s)(15, 19, 20). Incubation of HT29 cells with biotinylated hLAIR-1-IgG fusion proteins resulted in specific staining of the cells, whereas pre-incubation of these cells with unlabeled LAIR-2-IgG decreased the biotinylated hLAIR-1-IgG staining (FIG. 14A, top panel). In the reverse experiment, pre-incubation of HT29 cells with hLAIR-1-IgG reduced the staining with biotinylated LAIR-2-IgG (FIG. 14A, bottom panel). This demonstrates that both human LAIR-1 and LAIR-2 fusion proteins bind the same sites on the naturally expressed collagen molecules on HT29 cells.

Additionally, Oregon-green labeled collagen IV bound specifically to K562 cells transfected with hLAIR-1 (FIG. 14B, upper panel). This interaction was efficiently prevented by pre-incubation of the fluorescent collagen IV with soluble LAIR-2-IgG fusion proteins (FIG. 14B).

As documented before, hLAIR-1 transfected K562 cells bind firmly to plate-bound collagens I and III(15). Pre-incubation of plate-bound collagens I and III with LAIR-2-IgG fusion proteins blocked the binding of hLAIR-1 expressing K562 cells to collagens in a dose-dependent manner (FIG. 15A, left panel), while incubation with an irrelevant fusion protein had no effect (FIG. 15A, right panel). To assess whether wild-type LAIR-2 protein is capable of interfering with the hLAIR-1/collagen interaction, we concentrated supernatant from LAIR-2-transfected 293T cells and analyzed its blocking capacity in the above described adhesion assay. As expected, wild-type LAIR-2 present in the supernatant (FIG. 15B, left panel), but not control supernatant (FIG. 15B, right panel), blocked binding of stable human LAIR-1 expressing K562 cells to plate-bound collagen I and III. Thus LAIR-2 binds the same sites on various collagen species as hLAIR-1, showing that LAIR-2 functions as a soluble competitor to hLAIR-1/collagen binding in vitro.

LAIR-2Prevents hLAIR-1 Cross-Linking by Collagens

By using NFAT-GFP reporter cells expressing a chimeric protein containing the extracellular domain of hLAIR-1 and the transmembrane and intracellular domain of CD3ζ, we have shown that collagen I and III are capable to functionally cross-link hLAIR-1(15). We used this system to investigate whether LAIR-2-IgG can interfere with the activation of hLAIR-1-CD3ζ. Receptor engagement of cells expressing the hLAIR-1-CD3ζ chimera, but not the parental cells, via plate bound collagens I and III resulted in expression of GFP (FIG. 16). Pre-incubation of plate-bound collagen I and III with LAIR-2-IgG fusion proteins, but not isotype matched control IgG, abrogated the NFAT-GFP activation (FIG. 16), demonstrating that LAIR-2-IgG is also capable of blocking the functional interaction between hLAIR-1 and collagens.

In conclusion, five independent assays confirmed that LAIR-2 has the capacity to interfere with human LAIR-1 binding to various classes of collagen molecules.

LAIR-2 is Present in Inflamed Joints and Urine of Pregnant Women

To allow detection of wild-type LAIR-2 in human body fluids, we developed a LAIR-2 specific sandwich ELISA that was capable of detecting LAIR-2 in solution as low as 150 pg/ml (FIG. 17A), but showed no cross-reactivity for hLAIR-1 (data not shown). Soluble LAIR-2 was detected in the supernatant from 293T cells transiently transfected with LAIR-2, but not from untransfected cells (data not shown). The protein was not detected in plasma and serum from healthy individuals and pregnant women. Unexpectedly, LAIR-2 was present in large amounts in urine from pregnant women (average: 7108 pg/ml, range: 712-20877 pg/ml), whereas the antigen was not detected in urine of men and non-pregnant women (average: 209 pg/ml, range: 150 (below detection level)-695 pg/ml) (FIG. 17B). Although high levels of LAIR-2 were detected in urine, LAIR-2 levels in serum of pregnant women were still below detection level of our ELISA system (data not shown). Hence we conclude that LAIR-2 is produced as a soluble protein in vivo and that the molecule is cleared from the body via urine.

To explore whether LAIR-2 is present at sites of inflammation, we measured LAIR-2 in synovial fluid (SF) of patients with rheumatoid arthritis (RA, n=14) (FIG. 17C). LAIR-2 levels were compared to those found in SF from patients with osteoarthritis (OA, n=16) that suffer from joint degeneration with no to mild inflammation. LAIR-2 concentrations in SF from RA patients were elevated (average: 229 pg/ml, range: 150-318 pg/ml) as compared to that in OA patients (average: 165 pg/ml, range: 150-216 pg/ml). Thus, increased LAIR-2 levels in RA SF reflect the local inflammation in the joints of these patients.

Materials and Methods

Cells

Cell lines were obtained from the American Type Culture Collection and cultured using standard techniques. Cell lines used in this study: human monocytic U937 cells; HEK293T human embryonic kidney cells; human colon carcinomas lines HT29, DLD-1, LS174, SW480 and HCT116; SKBR3 human breast cancer line; YT.2C2 human NK-like cells; 721.221 lymphoblastoid cells; THP-1 human monocyte-like cells; Jurkat human T cells; CEM human leukemia T cells; and human erythroleukemia K562 cells. K562 cells and 2B4 NFAT-GFP T cell reporter cells (kindly provided by L. L. Lanier and H. Arase (USCF, USA)) were stably transfected with hLAIR-1 or hLAIR-1a-CD3ζ respectively as described previously(15).

Subjects

For LAIR-2 measurements in urine: 10 healthy individuals (5 males and 5 females) and 6 healthy pregnant women (16-37 weeks of gestation) volunteered in collecting their morning urine. The urine was stored at −80° C. prior use. For LAIR-2 measurements in synovial fluid (SF): from 14 patients suffering from rheumatoid arthritis (RA) and 16 patients suffering from osteoarthritis (OA) SF was obtained upon joint aspiration. The average age of the RA patients was 63.7 years (range 51-79). The average age of the OA patients was 56.0 years (range 34-87). Ten out of 16 RA patients were positive for rheumatoid factor. Samples were subjected to centrifugation to remove cells prior storage at −80° C.

Antibodies

The FMU-LAIR2.1 and FMU-LAIR2.3 IgG1 LAIR-2 mAbs were produced by immunization of BALB/c mice with recombinant LAIR-2 protein followed by preparation of hybridomas by using standard hybridoma techniques. Selected hybridomas were subcloned by limiting dilution, and mAbs were purified by affinity chromatography on protein A-Sepharose columns (Amersham, Freiburg, Germany). Biotinylated anti-human LAIR-2 antibody was obtained from R&D systems (BAF2665).

RT PCR

Total RNA was isolated from several human cell lines using the TRIzol method according to the manufacturer's instructions (Invitrogen). Total RNA was converted to first-strand DNA with oligo(dT)₂₀ primers and M-MLV reverse transcriptase using the SuperScript™ III First-Strand Synthesis System for RT-PCR (Invitrogen). The cDNA mixtures were amplified by PCR using LAIR-2 specific forward (5′-GTTGGGGTTCAAACATTCCG-3′) and reverse (5′-TCATGGTGCATCAAATCCGG-3′) primers and the AmpliTaq Gold DNA polymerase system (PE Applied Biosystems). Each amplification reaction underwent 40 cycles of denaturation at 95° C. for 30 s, annealing for 30s at 54° C. and elongation at 72° C. for 50 s. As a control, GAPDH transcripts were amplified from the same RNA, using GAPDH specific forward (5′-GGTACATGACAAGGTGCGGC-3′) and reverse (5′-GCATCCTGGGCTACACTGAGC-3′) primers.

The LAIR-2 isoforms were cloned using the pGEM-T easy vector system (Promega, Madison, Wis.) and sequenced on an ABI 3100 sequencer (PE Applied Biosystems) using the ABI PRISM BigDye Terminator Cycle Sequencing Ready Reaction kit (PE Applied Biosystems). The sequences were analyzed by Lasergene software (DNASTAR, Londen, UK).

Western Blot Analysis

293T cells were transiently transfected with a vector encoding LAIR-2b and cultured in Optimem medium (Gibco, Breda, Netherlands), 48 hours after transfection the supernatant was collected and separated by SDS-polyacrylamide gel electrophoresis (12% gel) under reducing conditions and transferred to Immobilon-P membranes (Millipore, Bedford, Mass.). Western blot analysis was performed with FMU-LAIR2.1 anti-LAIR-2 mAbs, followed by peroxidase-conjugated rabbit anti mouse mAb (DAKO) as secondary antibody. Proteins were detected by enhanced chemiluminescence (GE, UK).

Surface Plasmon Resonance

The surface plasmon resonance binding studies were performed as described before(15). LAIR-2-IgG dimer concentration was calculated based on a theoretical mass of 82.5 kDa.

Flow Cytometry

For intracellular LAIR-2 staining, the Zenon Mouse IgG Labeling Kit (Molecular Probes) was used according to the manufacturer's instructions. Cells were analyzed by flow cytometry (FACSCalibur™, BD).

Interaction of Oregon green 488-conjugated collagen IV (Molecular Probes) with hLAIR-1 or LAIR-2 was performed as follows; collagen IV was incubated with 40 μg/ml LAIR-2-IgG or control fusion protein for 30 minutes at room temperature. Subsequently, the mixture was added to K562 cells stably transfected with hLAIR-1 or wild-type K562 cells for 30 minutes, washed and cells were analyzed by flow cytometry (FACSCalibur™, BD).

For fusion protein blocking studies, chimeric proteins of the extracellular domain of hLAIR-1 or LAIR-2 fused to the Fc region of human IgG1 were prepared as described previously(19). HT29 cells were pre-treated with 10% normal mouse serum to block aspecific interactions. Subsequently, HT29 cells were incubated with unlabeled hLAIR-1-IgG or LAIR-2-IgG for 10 minutes at room temperature and subsequently incubated with biotinylated hLAIR-1-IgG or LAIR-2-IgG for 30 minutes at room temperature. Binding of biotinylated proteins was detected with allophycocyanin (APC) conjungated streptavidin (BD Biosciences, San Diego, Calif.) and cells were analyzed by flow cytometry (FACSCalibur™, BD).

Binding of K562 Transfectants to Plate-Bound Collagen

96-well MAXIsorp flat-bottom plates (Nunc) were coated overnight at 4° C. with purified collagens I or III (Sigma, 2 and 5 μg/ml respectively) or BSA (5 μg/ml) in 100 μl PBS, supplemented with 2 mM acetic acid. 5×10⁶ cells/ml wild-type K562 or K562 stably transfected with hLAIR-1 were assayed for their capacity to adhere to the collagens in the 96 well plates as described before(15).

Reporter Cell Assay

2B4 T cell hybridoma cells were stably transduced with an NFAT-GFP reporter construct(40) and hLAIR-1-CD3ζ(15) and analyzed as described(15, 40).

Sandwich ELISA

96-well flat bottom MAXIsorp plates (Nunc) were coated overnight at 4° C. with FMU-LAIR2.1 anti-LAIR-2 mAbs (6 μg/ml in 50 μl/well PBS). After washings, the plates were incubated with 3% BSA in PBS to block aspecific interactions. Meanwhile, synovial fluid (SF) were treated with hyaluronidase type IV, 20 units/ml (Sigma, Munich, Germany), for 20 minutes at 37° C. to reduce viscosity. After three washes of the plate, supernatants or biological samples (urine or SF) were assayed for presence of the LAIR-2 protein. Human recombinant LAIR-2 (R&D systems) serially diluted from 200 ng/ml was used as a reference protein. Samples were prepared in PBS containing 3% BSA and incubated for two hours at room temperature. After three washes, the wells were incubated with a biotinylated LAIR-2 mAb (R&D systems) for two hours at room temperature. After washings, the wells were incubated with StreptABComplex/HRP (Dako) for one hour and color development was performed by adding 100 μl/well ABTS reagent (Roche Diagnostics). A Mann-Whitney test was performed to calculate statistical significance between the separate groups. Samples which-had no detectable LAIR-2 were included in the statistical analysis; these were given the value of the detection limit (150 pg/ml). Since rheumatic factor could interfere with the LAIR-2 specific ELISA in RA SF samples, control measurements were included by replacing the anti-LAIR-2 capture antibody with an isotype-matched control antibody (BD Biosciences, San Diego, Calif.). The subsequent protocol was identical as described above. This isotype-matched control measurement did not result in a specific signal higher than background measurements.

### REFERENCE LIST OF EXAMPLE 4

1. Lanier, L. L. (2005) Annu. Rev. Immunol. 225-274.
2. Ravetch, J. V. & Lanier, L. L. (2000) Science 290, 84-89.
3. Verbrugge, A., de Ruiter, T., Geest, C., Coffer, P. J. & Meyaard, L. (2006) J. Leukoc. Biol. 79, 828-836.
4. Maasho, K., Masilamani, M., Valas, R., Basu, S., Coligan, J. E. & Borrego, F. (2005) Mol. Immunol. 42, 1521-1530.
5. Bennett, F., Luxenberg, D., Ling, V., Wang, I. M., Marquette, K., Lowe, D., Khan, N., Veldman, G., Jacobs, K. A., Valge-Archer, V. E. et al. (2003) J. Immunol. 170, 711-718.
6. Moller, M. J., Kammerer, R., Grunert, F. & von Kleist, S. (1996) Int. J. Cancer. 65, 740-745.
7. Levine, S. J. (2004) J Immunol. 173, 5343-5348.
8. Ilan, N. & Madri, J. A. (2003) Curr. Opin. Cell Biol. 15, 515-524.
9. Meyaard, L., Adema, G. J., Chang, C., Woollatt, E., Sutherland, G. R., Lanier, L. L. & Phillips, J. H. (1997) Immunity. 7, 283-290.
10. Poggi, A., Tomasello, E., Ferrero, E., Zocchi, M. R. & Moretta, L. (1998) Eur. J. Immunol. 28, 2086-2091.
11. van der Vuurst de Vries A R, Clevers, H., Logtenberg, T. & Meyaard, L. (1999) Eur. J. Immunol. 29, 3160-3167.
12. Poggi, A., Pella, N., Morelli, L., Spada, F., Revello, V., Sivori, S., Augugliaro, R., Moretta, L. & Moretta, A. (1995) Eur. J. Immunol. 25, 369-376.
13. Meyaard, L., Hurenkamp, J., Clevers, H., Lanier, L. L. & Phillips, J. H. (1999) J. Immunol. 162, 5800-5804.
14. Saverino, D., Fabbi, M., Merlo, A., Ravera, G., Grossi, C. E. & Ciccone, E. (2002) Hum. Immunol. 63, 534-546.
15. Lebbink, R. J., de Ruiter, T., Adelmeijer, J., Brenkman, A. B., van Helvoort, J. M., Koch, M., Farndale, R. W., Lisman, T., Sonnenberg, A., Lenting, P. J. et al. (2006) J. Exp. Med. 203, 1419-1425.
16. Myllyharju, J. & Kivirikko, K. I. (2004) Trends Genet. 20, 33-43.
17. Verbrugge, A., de Ruiter, T., Geest, C., Coffer, P. J. & Meyaard, L. Differential expression of Leukocyte Associated Ig-like Receptor-1 during neutrophil differentiation and activation. (2006) J.Leukoc.Biol. 79, 828-36.
18. Ouyang, W., Xue, J., Liu, J., Jia, W., Li, Z., Xie, X., Liu, X., Jian, J., Li, Q., Zhu, Y. et al. (2004) J Immunol. Methods 292, 109-117.
19. Lebbink, R. J., de Ruiter, T., Verbrugge, A., Bril, W. S. & Meyaard, L. (2004) J. Immunol. 172, 5535-5543.
20. Lebbink, R. J., de Ruiter, T., Kaptijn, G. J. & Meyaard, L. (2005) Immunogenetics 57, 344-351.
21. Ricard-Blum, S. & Ruggiero, F. (2005) Pathol. Biol. (Paris) 53, 430-442.
22. Knight, C. G., Morton, L. F., Onley, D. J., Peachey, A. R., Ichinohe, T., Okuma, M., Farndale, R. W. & Barnes, M. J. (1999) Cardiovasc. Res. 41, 450-457.
23. Heaney, M. L. & Golde, D. W. (1996) Blood 87, 847-857.
24. Colonna, M., Navarro, F., Bellon, T., Llano, M., Garcia, P., Samaridis, J., Angman, L., Cella, M. & Lopez-Botet, M. (1997) J. Exp. Med. 186, 1809-1818.
25. Arm, J. P., Nwankwo, C. & Austen, K. F. (1997) J Immunol. 159, 2342-2349.
26. Borges, L., Hsu, M. L., Fanger, N., Kubin, M. & Cosman, D. (1997) J Immunol. 159, 5192-5196.
27. Gomez-Lozano, N., Estefania, E., Williams, F., Halfpenny, I., Middleton, D., Solis, R. & Vilches, C. (2005) Eur. J Immunol. 35, 16-24.
28. Borges, L., Kubin, M. & Kuhlman, T. (2003) Blood 101, 1484-1486.
29. Baylis, C. (1999) Semin. Nephrol. 19, 133-139.
30. Zhang, J. G., Hilton, D. J., Willson, T. A., McFarlane, C., Roberts, B. A., Moritz, R. L., Simpson, R. J., Alexander, W. S., Metcalf, D. & Nicola, N. A. (1997) J. Biol. Chem. 272, 9474-9480.
31. Arntzen, K. J., Liabakk, N. B., Jacobsen, G., Espevik, T. & Austgulen, R. (1995) Am. J Reprod. Immunol. 34, 163-169.
32. Hoffmann, J. C., Dengler, T. J., Knolle, P. A., Albert-Wolf, M., Roux, M., Wallich, R. & Meuer, S. C. (1993) Eur. J. Immunol. 23, 3003-3010.
33. Novick, D., Engelmann, H., Wallach, D. & Rubinstein, M. (1989) J. Exp. Med. 170, 1409-1414.
34. Marcon, L., Fritz, M. E., Kurman, C. C., Jensen, J. C. & Nelson, D. L. (1988) Clin. Exp. Immunol. 73, 29-33.
35. Hintzen, R. Q., de Jong, R., Hack, C. E., Chamuleau, M., de Vries, E. F., ten Berge, I. J., Borst, J. & van Lier, R. A. (1991) J. Immunol. 147, 29-35.
36. Hefler, L., Kainz, C., Zeisler, H., Heinze, G., Schatten, C., Husslein, P., Leodolter, S. & Tempfer, C. (1999) Acta Obstet. Gynecol. Scand. 78, 580-585.
37. Sargent, I. L., Borzychowski, A. M. & Redman, C. W. (2006) Trends Immunol.
38. Feldmann, M., Brennan, F. M. & Maini, R. N. (1996) Cell. 85, 307-310.
39. Hunter, D. J. & Felson, D. T. (2006) BMJ. Mar. 18, 2006;332. (7542.):639.-42. 332, 639-642.
40. Voehringer, D., Rosen, D. B., Lanier, L. L. & Locksley, R. M. (2004) J. Biol. Chem. 279, 54117-54123.

### Figure Legends

FIG. 1. Collagen XVII is a ligand for LAIR-1.

(A) Ba/F3 cells transfected with human collagen XVII (filled histograms) or the parental cell line (open histograms) were stained with indicated LAIR-fusion proteins or anti-collagen XVII antibodies. (B) anti-human LAIR-1 antibodies (8A8) completely abrogate the human collagen XVII/hLAIR-1-IgG-interaction. (C) polyclonal anti-mouse LAIR-1 antibodies abrogate human collagen XVII/mLAIR-1-IgG-interaction. All experiments are representative of three independent experiments.

FIG. 2. Collagen XVII and LAIR-1 transfected cells form aggregates.

(A) Conjugate analysis. K562 cells transfected with/without hLAIR-1, mLAIR-1 or human collagen XVII were either red or green fluorescently labeled, co-incubated in various indicated combinations at 37° C. and analyzed by FACS (percent of double positive cell conjugates is indicated) or (B), visual inspection for cell clustering. All experiments are representative of three independent experiments.

FIG. 3. LAIR-1 is a collagen receptor.

(A) SK-BR3 cells express a collagen ligand for LAIR-1 that is not collagen XVII. HT29 or SK-BR3 cells were stained with indicated proteins with or without pre-treatment with collagenase. Filled histograms represent staining with LAIR-1-IgG or anti-collagen XVII mAb, open histograms represent control staining. (B) 293T cells were transiently transfected using FuGENE6 (Sigma) according to the manufacturers protocol with control vector (open histograms) or indicated HIS-tagged collagens (gray histograms) and subsequently stained with hLAIR-1-IgG (top panels) or anti-HIS-antibody (bottom panels). Similar results were obtained using hLAIR-2-IgG, mLAIR-1-IgG, and rLAIR-1-IgG. (C) Fluorescently labeled wt K562 cells (open bars) or K562 cells expressing hLAIR-1 (black bars) or KIR3DL1 (gray bars) were monitored for their capacity to bind immobilized collagens I, II and III. Where indicated, cells were pre-incubated with anti-hLAIR-1 F(ab′)₂ (8A8) fragments (hatched bars). Two other members of the IgSF (CD48 and CD80 transfected in K562 cells) did not associate with collagens (data not shown). Percentage of adhering cells relative to input is shown. One of three independent experiments is shown. (D) mLAIR-1-transfected K562 cells spread upon interaction with immobilized collagen I. Spreading was also observed upon interaction with immobilized (GPO)₁₀, but not (GPP)₁₀ (data not shown). Non-transfected cells did not adhere to collagen I or (GPO)₁₀ (data not shown). (E) Immunoprecipitation using mLAIR-1-IgG, hLAIR-1-IgG or control protein with (+) or without (−) purified hcollagen III. Interacting proteins were Western blotted using anti-hcollagen III specific mAbs. Positive control represents purified collagen III. (F) Cell-lysates from human PBMCs or hLAIR-1-transfected K562 were pre-incubated with or without anti-hLAIR-1 F(ab′)2 fragments before incubation with insoluble collagen I fibrils. Collagen fibrils and interacting proteins were centrifuged, washed and Western blotted using anti-hLAIR-1 mAbs. (G) Tissue sections of human skin stained with biotinylated mLAIR-1-IgG (middle panel) or control Ig (left panel) followed by streptavidin-HRP and counterstaining with hematoxylin. Staining was blocked by pre-incubation of mLAIR-1-IgG with purified collagen I (right panel). Staining with hLAIR-Ig gave similar results.

FIG. 4. LAIR-1 is a high-affinity collagen receptor.

(A) Indicated concentrations hLAIR-1-IgG were injected at 20 μl/min sequentially through a BIAcore flow cell containing ˜2000-3000 RU of directly immobilized collagen I (∘), III (□) or nothing. Each symbol represents the resonance unit at equilibrium and corresponding concentration; the graph was used to determine the indicated Kd-values. Control fusion-protein did not associate with collagen. (B) Rate of dissociation of hLAIR-1-IgG from collagen I and III as monitored by surface plasmon resonance. (C) K562-cells transfected with human LAIR-1 bind collagen III under flow conditions. Indicated K562 transfectants were perfused at wall shear rates of 0.76 dynes/cm² over collagen III-coated coverslips. The number of binding cells was counted after 5 minutes (mean ±SEM, n=6). (D) LAIR-1 binds immobilized (GPO)₁₀, but not (GPP)₁₀. hLAIR-1-IgG (780 nM) was injected at 5 μl/min through a BIAcore flow cell containing ˜250 RU of indicated immobilized (GPO)₁₀ or (GPP)₁₀.

FIG. 5. Collagen I and (GPO)₁₀ directly inhibit degranulation of RBL-2H3 cells via LAIR-1.

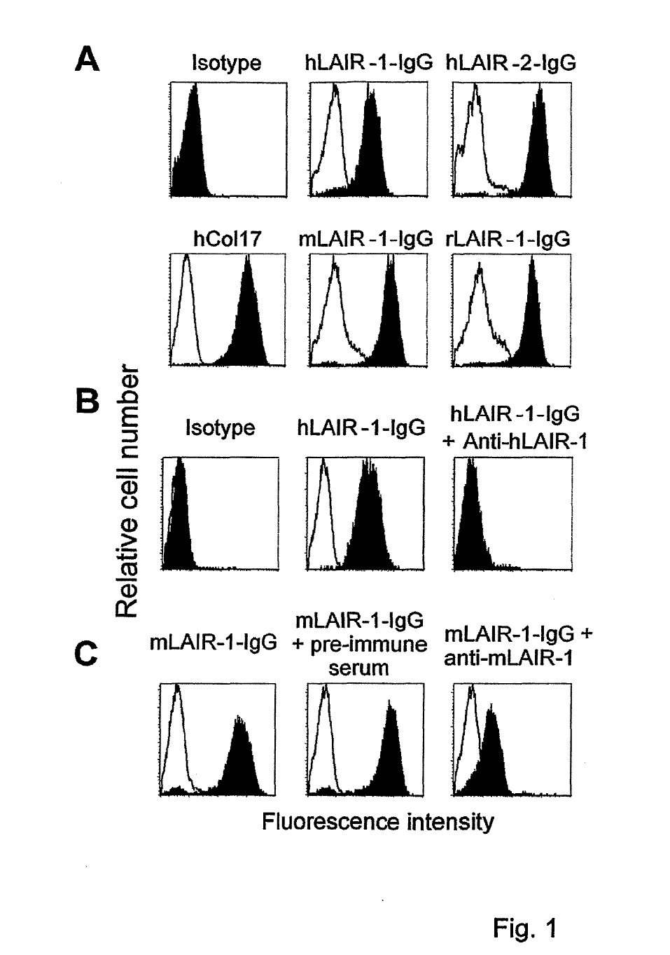

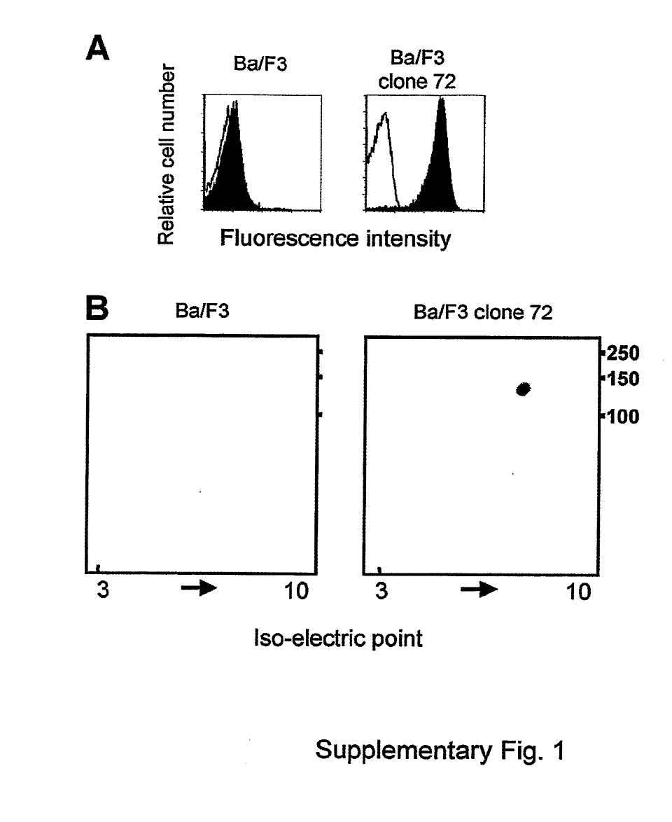

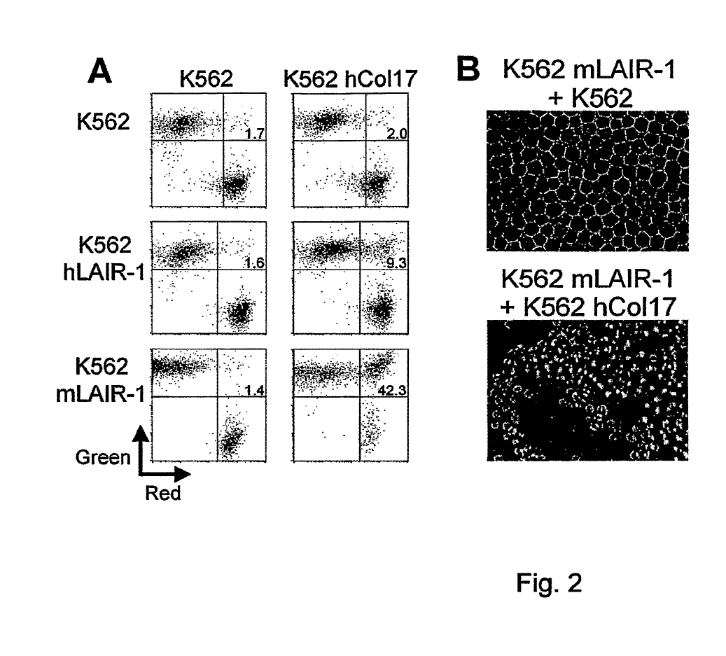

(A) NFAT-GFP reporter cells¹⁷ transfected with hLAIR-1-CD3ζ chimera (bottom panels) or not (top panels) were incubated with immobilized collagens I, III, BSA, or anti-hLAIR-1 mAbs for 20 hours and GFP expression was analyzed by flow cytometry. Percentage of GFP-positive cells is indicated. (B) hLAIR-1 transfected RBL-2H3⁷ cells were sensitized with IgE anti-TNP, incubated at 37° C. in plates coated with TNP conjugated to BSA (0.8 μg/ml) in the absence or presence of plate bound collagen I, III, or anti-hLAIR-1 mAb (3.3 μg/ml, closed bars). Where indicated cells were pre-treated with 50 μg/ml anti-hLAIR-1 F(ab′)₂ fragments (8A8, open bars). (C) The same experiment was performed using RBL-2H3 cells transfected with a LAIR-1 mutant in which both tyrosines in the intracellular tail are mutated to fenylalanines (hLAIR-1 FF). (D) hLAIR-1 transfected RBL-2H3 cells were sensitized with IgE anti-TNP, incubated at 37° C. in plates coated with TNP conjugated to BSA (0.8 μg/ml ) in the presence of plate-bound trimeric (GPO)₁₀ or (GPP)₁₀ peptides (coated at 3.3 μg/ml) (closed bars). Where indicated cells were pre-treated with 50 μg/ml anti-hLAIR-1 F(ab′)₂ fragments (8A8, open bars). Supplementary FIG. 1. Identification of mouse collagen XVII as a ligand for mouse LAIR-1.

(A) Selection of LAIR-1L expressing clones. Ba/F3 cells were stably transfected with a mouse retroviral cDNA library and cells expressing LAIR-ligand were selected by sorting using magnetic beads coated with mLAIR-1-IgG and subsequently cloned by limiting dilution. Binding of mLAIR-1-IgG to a representative clone (Ba/F3 clone 72) and the parent cell line (Ba/F3) is shown. Filled histograms represent staining with mLAIR-1-IgG and open histograms represent isotype staining. (B) Identification of mouse collagen XVII as ligand for mouse LAIR-1 by immunoprecipitation. Ba/F3 cells or Ba/F3 cells expressing mLAIR-ligand were surface biotinylated, lysed and subjected to immunoprecipitation using mLAIR-1-IgG-coated protein A/G-beads. Interacting proteins were separated by two-dimensional gel electrophoresis, transferred to Immobilon-P membranes and Western blotted using HRP-conjugated streptavidin. A single biotinylated molecule with a molecular mass of ˜140 kD and an isoelectric point of ˜8 present in the LAIR-ligand-positive clone was subjected to protein sequencing by mass-spectrometry and proved identical to mouse collagen XVII.

FIG. 6. Cross-linking of LAIR-1 results in inhibition of primary T-cell function.

PBMC were stimulated with αCD3 PBMC were stimulated with αCD3 in the presence of 0.3 μg/ml collagen 1 or BSA (C). The number of positive spots per 100.000 cells was measured and the production in the presence of BSA control was set at 100%. Relative production of IFNγ in the presence of collagen 1 was calculated at several concentrations of stimulus. Median values together with the 25^{th}-75^{th} percentiles of three independent experiments are shown.

FIG. 7. Identification of multiple LAIR-1 binding sites on human collagen II.

A: K562 cell adhesion assay. Fluorescently labeled K562 cells stably expressing human LAIR-1 were monitored for their capacity to bind immobilized synthetic trimeric peptides encompassing the entire triple-helical domain of human collagen II (Collagen II Toolkit). Percentage of adhering cells relative to input is shown.

B: 2B4 NFAT-GFP reporter cell assay. 2B4 NFAT-GFP reporter cells stably transfected with hLAIR-1-CD3ζ chimeric molecules were incubated with the indicated immobilized collagens II synthetic trimeric peptides for 22 hours and GFP expression was analyzed by flow cytometry. Maximal stimulation in these reporter cell assays using trimeric collagen peptides typically resulted at most in ˜70% GFP⁺ cells. Parental K562 cells and 2B4 NFAT-GFP reporter cells did not adhere or respond to any of the peptides. The sequences of the indicated peptides are shown in supplemental table I. Data are expressed as mean values of three independent experiments plus standard deviation.

FIG. 8. Identification of multiple LAIR-1 binding sites on human collagen III.

A: K562 cell adhesion assay. Fluorescently labeled K562 cells stably expressing human LAIR-1 were monitored for their capacity to bind immobilized synthetic trimeric peptides encompassing the entire triple-helical domain of human collagen III (Collagen III Toolkit). Percentage of adhering cells relative to input is shown.

B: 2B4 NFAT-GFP reporter cell assay. 2B4 NFAT-GFP reporter cells stably transfected with hLAIR-1-CD3ζ chimeric molecules were incubated with the indicated immobilized collagens III synthetic trimeric peptides for 22 hours and GFP expression was analyzed by flow cytometry. Maximal stimulation in these reporter cell assays using trimeric collagen peptides typically resulted at most in ˜70% GFP⁺ cells. Parental K562 cells and 2B4 NFAT-GFP reporter cells did not adhere or respond to any of the peptides. The sequences of the indicated peptides are shown in supplemental table II. Data are expressed as mean values of three independent experiments plus standard deviation.

FIG. 9. Peptides II-56 and III-30 are potent inhibitors of CD3-induced T cell activation via hLAIR-1

Human LAIR-1 transfected (closed squares) or untransfected (open squares) NFAT-GFP reporter T cells were incubated with immobilized anti-CD3 mAbs and indicated synthetic trimeric peptides for 22 hours and GFP expression was analyzed by flow cytometry. Percentage inhibition of anti-CD3-induced GFP expression is presented as a function of peptide concentration during coating (see materials and methods). Typically, ˜90 percent of the reporters expressed GFP upon CD3 stimulation alone. The peptide concentrations used in the experiments were: 10, 3.3, 1.1, and 0.37 μg/ml. The percentage of inhibition of CD3 stimulation was calculated as follows: percentage of inhibition=100*[(% GFP⁺ cells in BSA coated well−% GFP⁺ cells in peptide coated well)/(% GFP⁺ cells in BSA coated well)].

FIG. 10. Peptides II-56 and III-30 are potent inhibitors of FcεRI-induced degranulation of RBL-2H3 cells

Human LAIR-1 transfected (closed squares) or untransfected (open squares) RBL-2H3 (23) cells were sensitized with IgE anti-TNP and subsequently incubated at 37° C. in plates coated with TNP conjugated to BSA (0.8 μg/ml) and either 1, 0.75, 0.5, 0.3 μg/ml synthetic trimeric peptides. Degranulation was measured as described before (23). Typical degranulation values ranged from 10 to 25% of the amount observed when all RBL cells were lysed by addition of 10% Triton. The relative percentage of degranulation was calculated as: 100*[(OD₄₀₅ TNP+peptide−OD₄₀₅ spontaneous release)/(OD₄₀₅ TNP alone−OD₄₀₅ spontaneous release)]. Measurements were performed using triplicate cultures. One representative experiment of two is shown.

FIG. 11. Increased GPO-content in peptides correlates with increased binding potential to LAIR-1

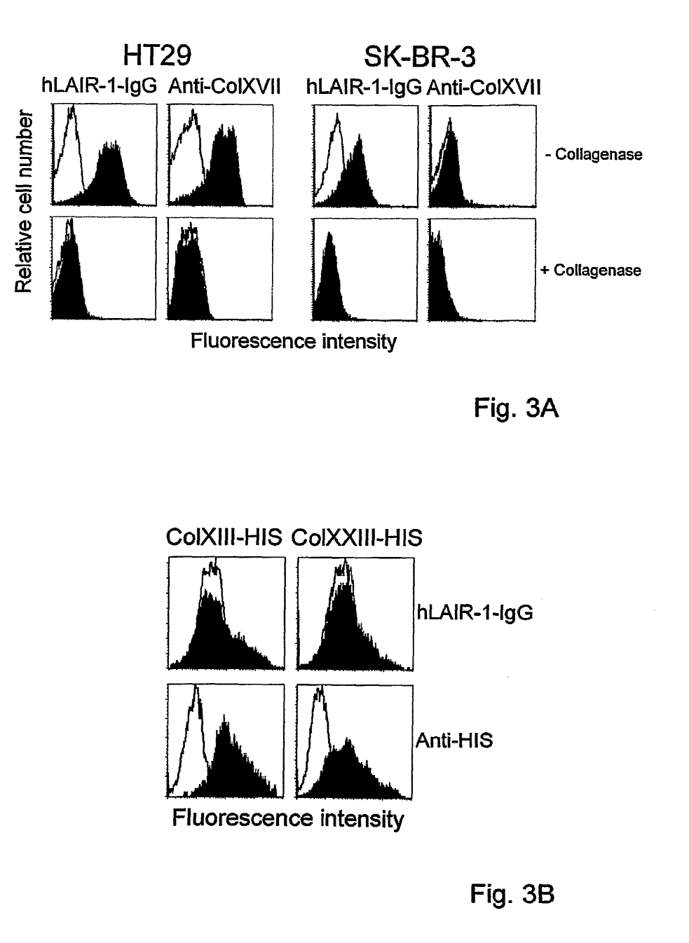

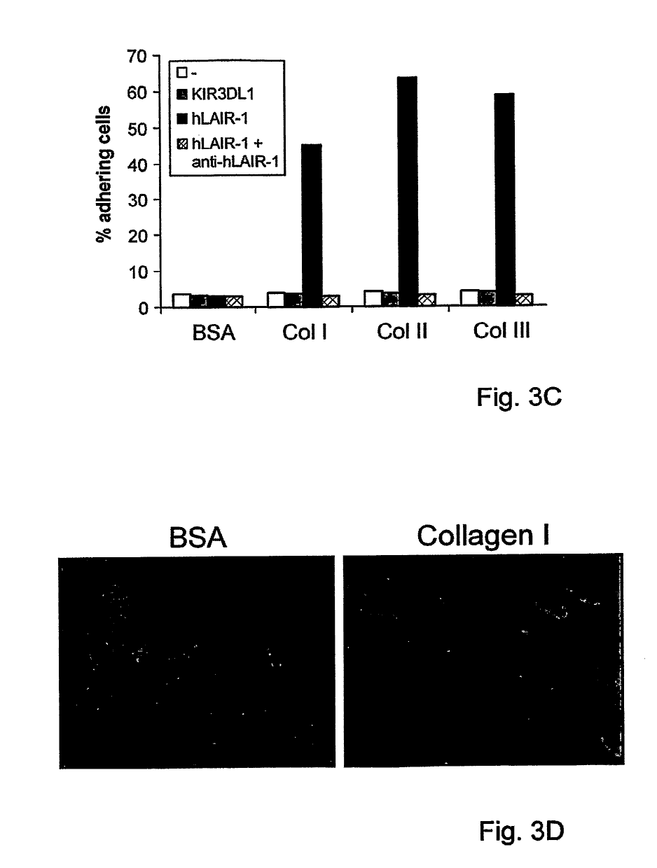

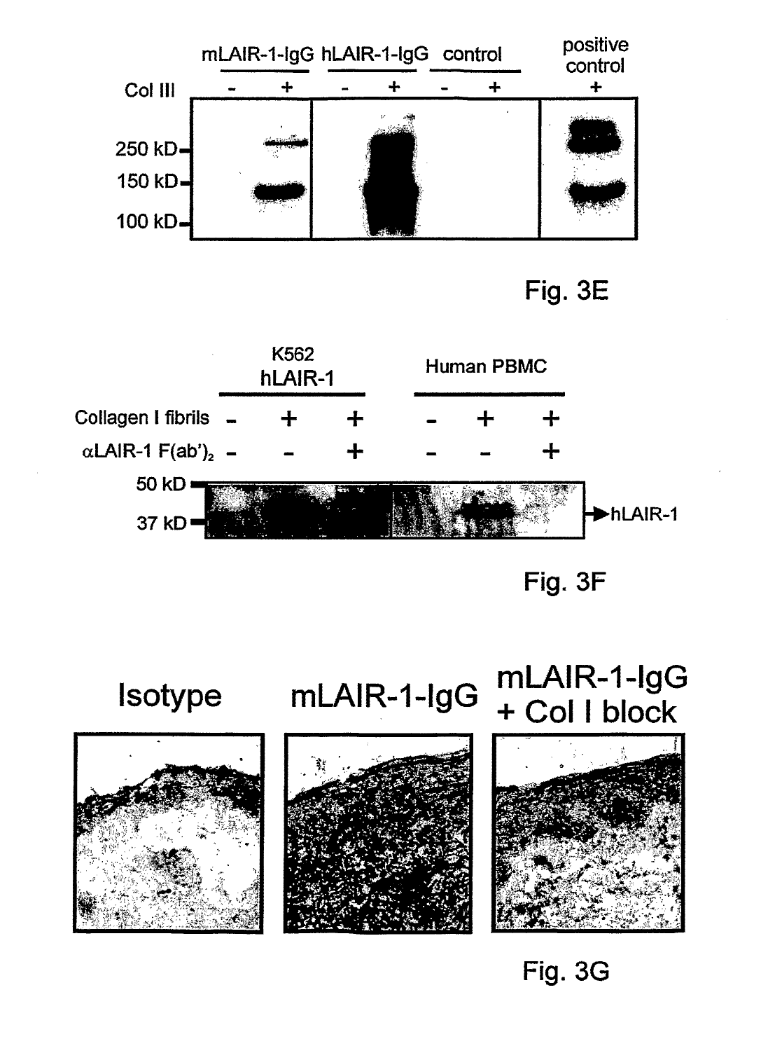

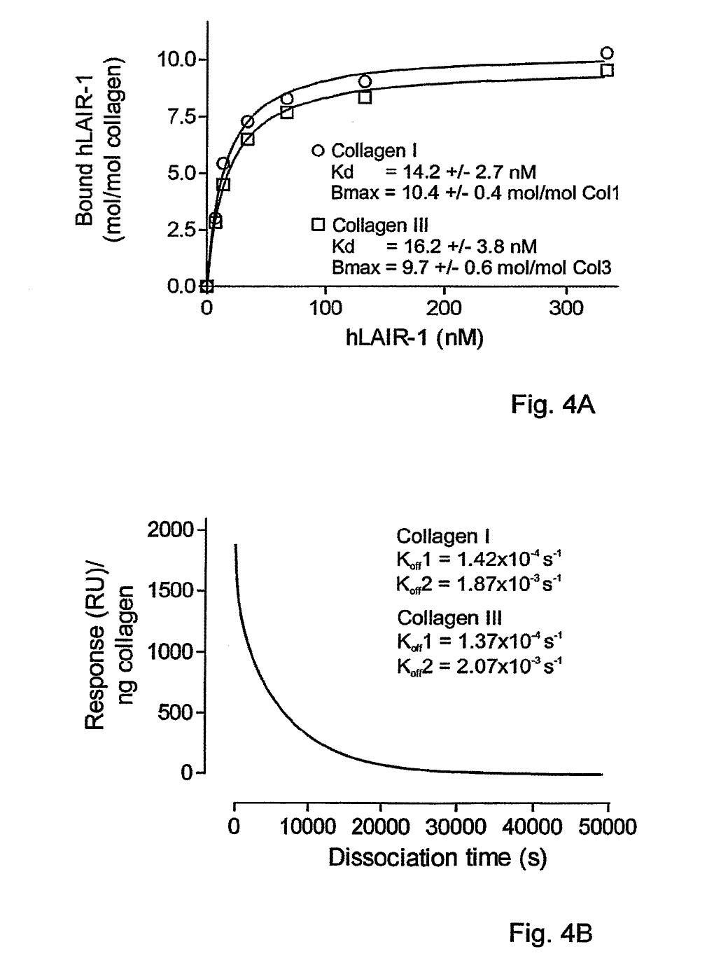

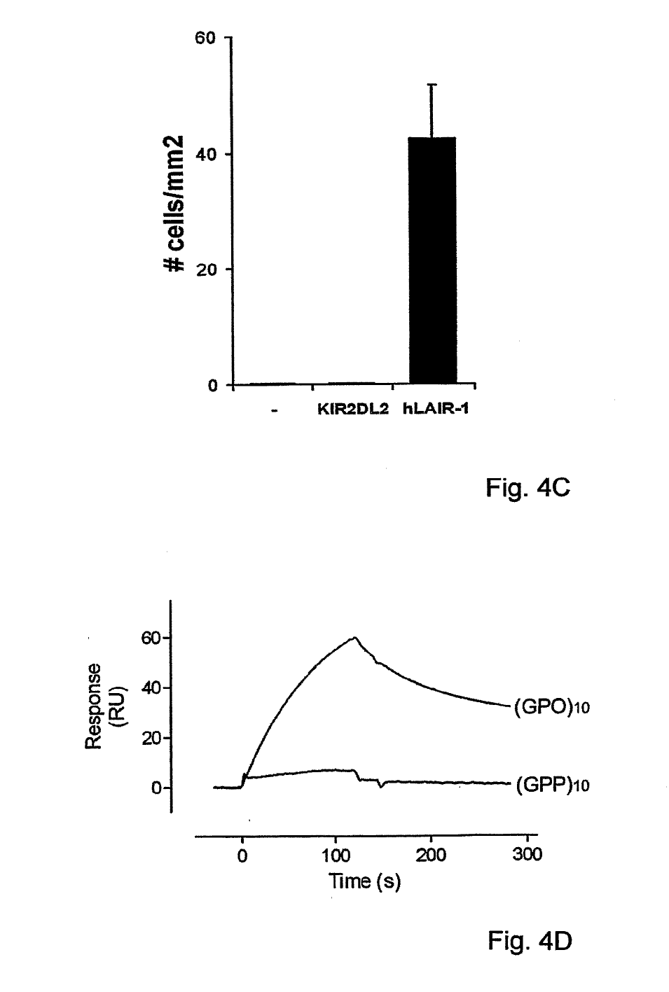

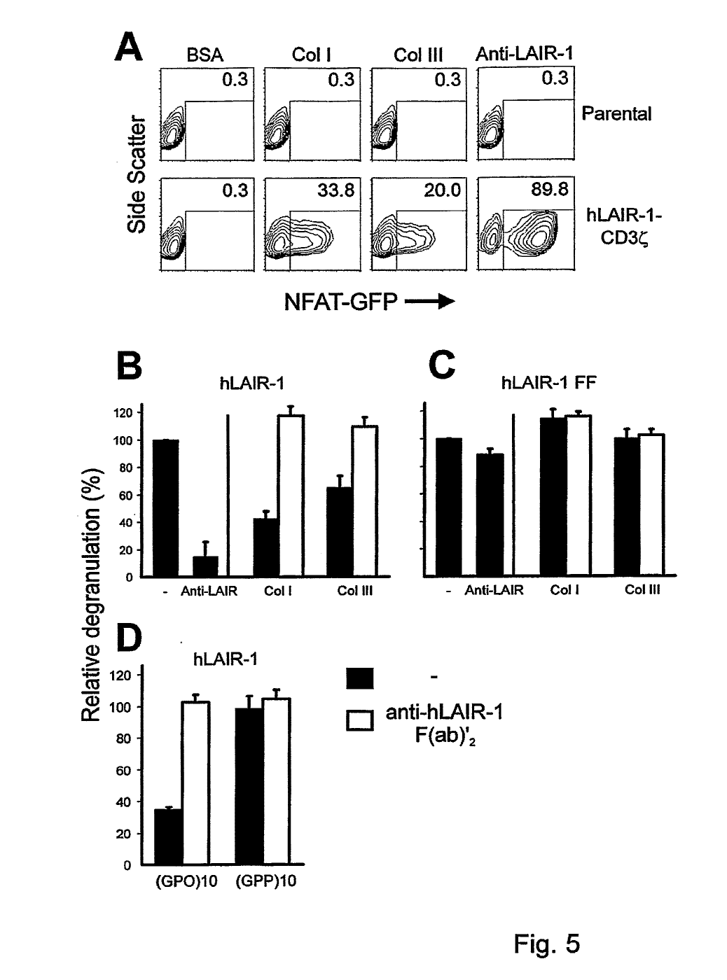

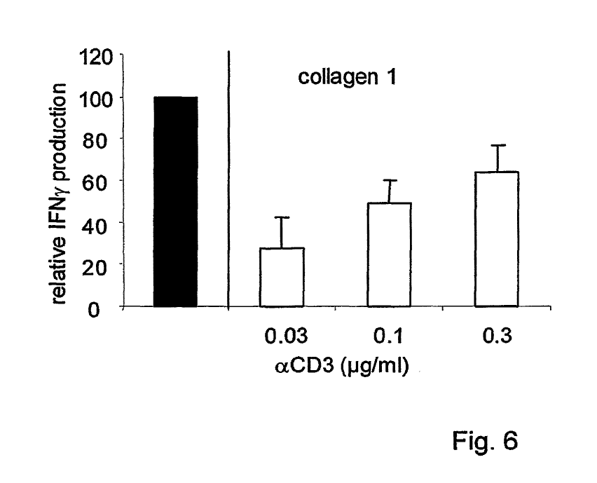

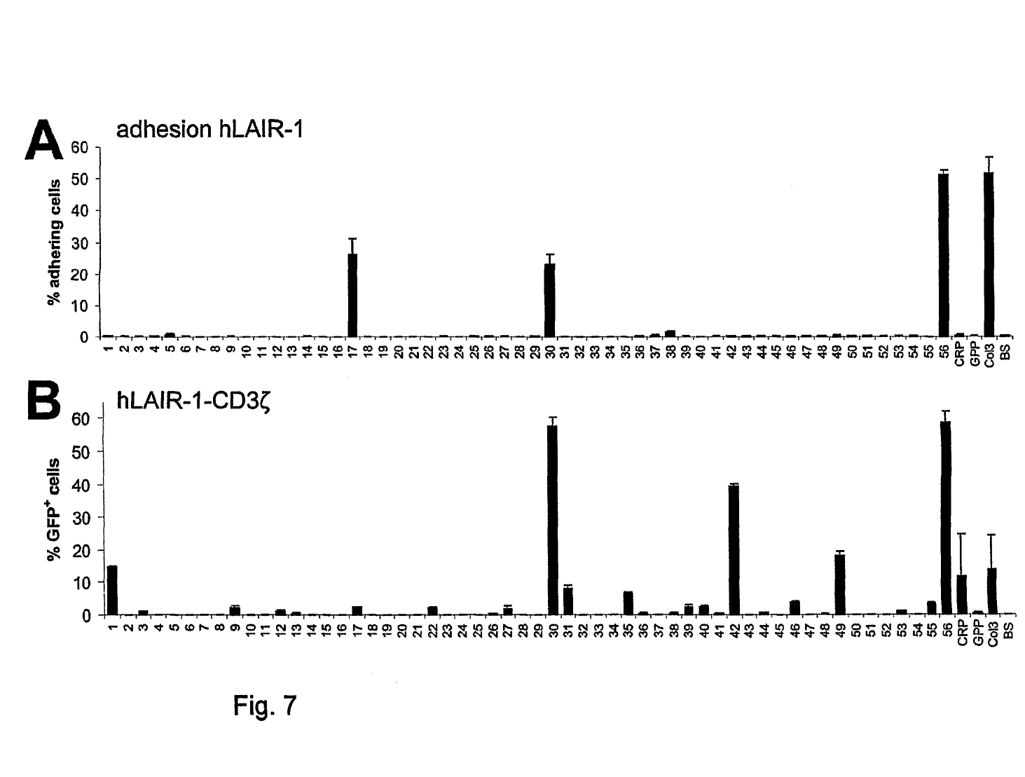

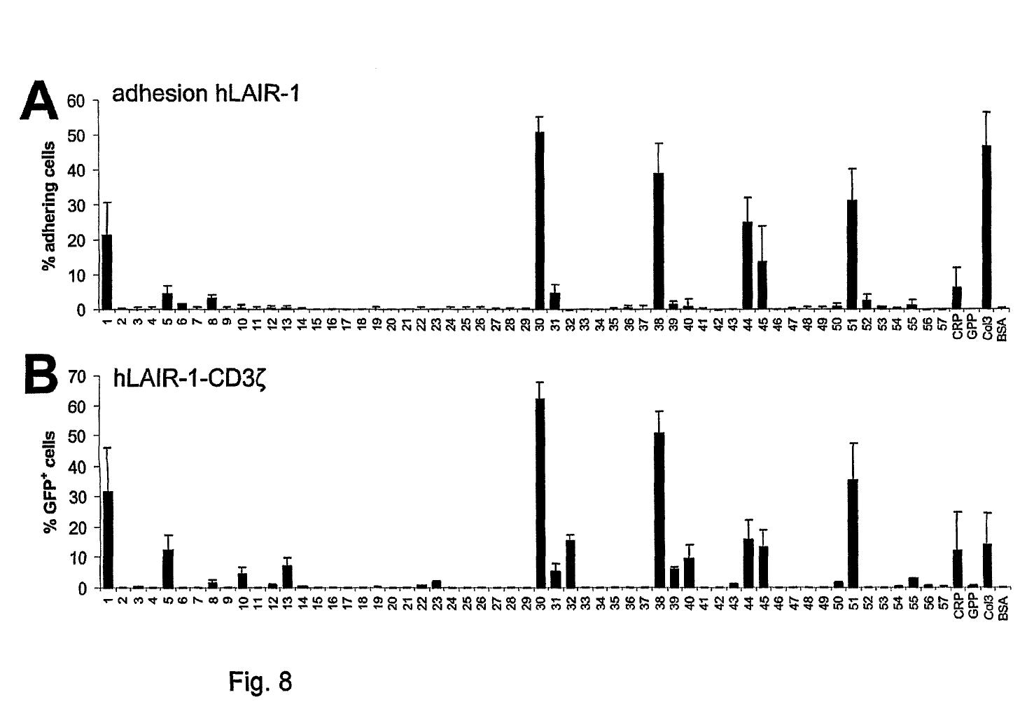

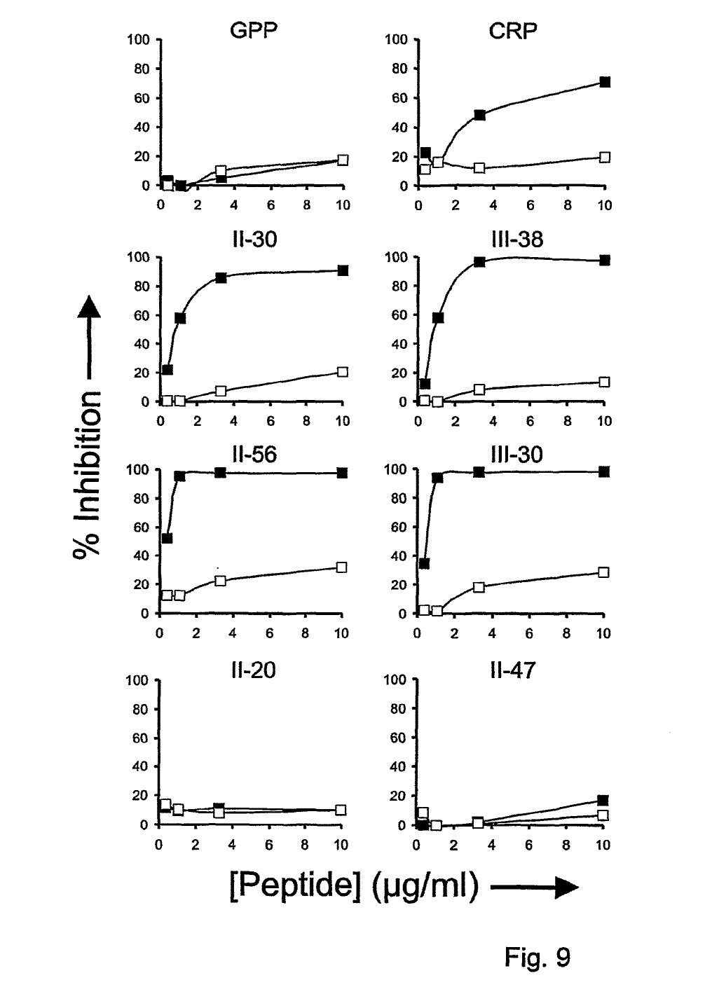

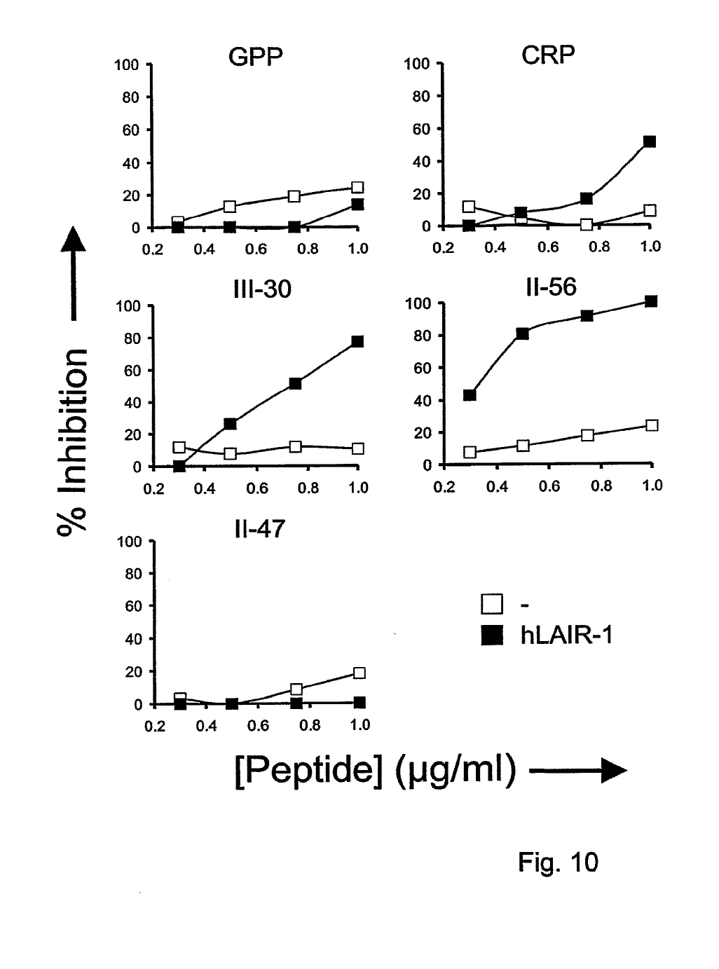

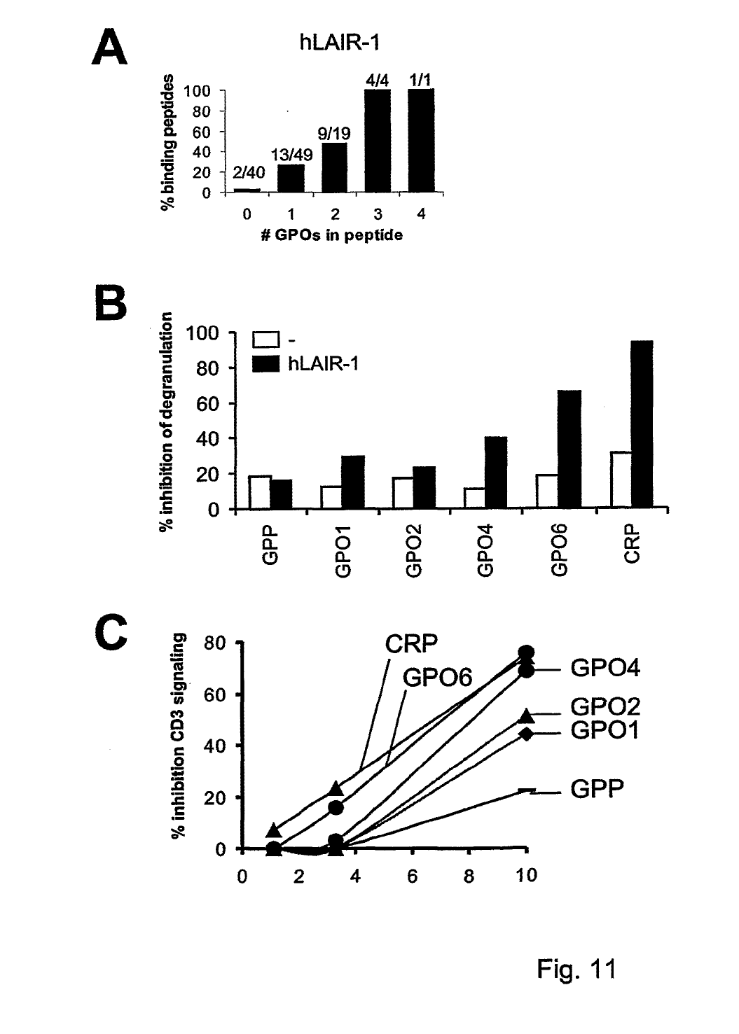

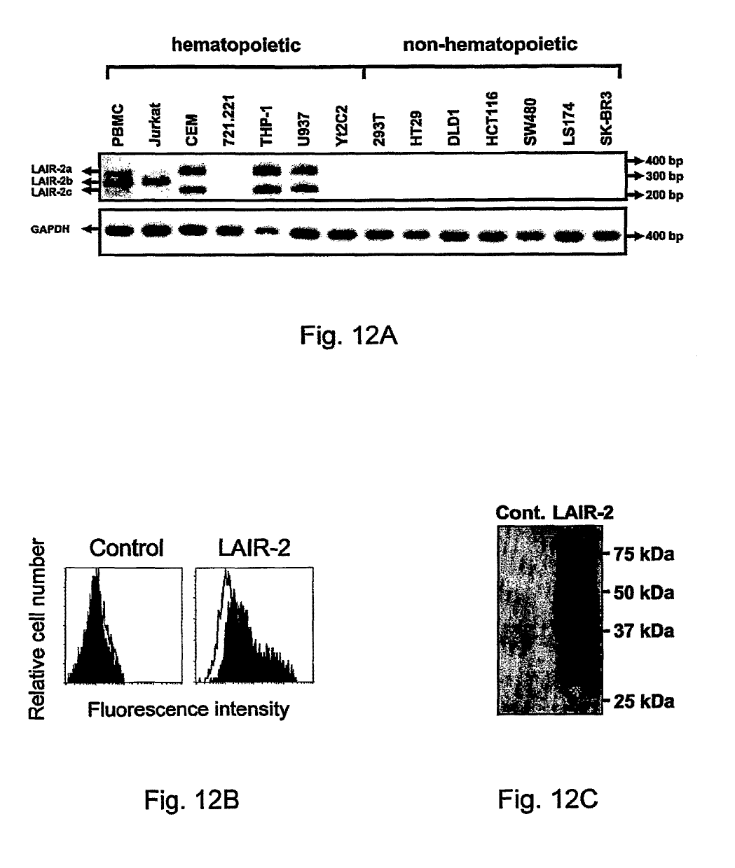

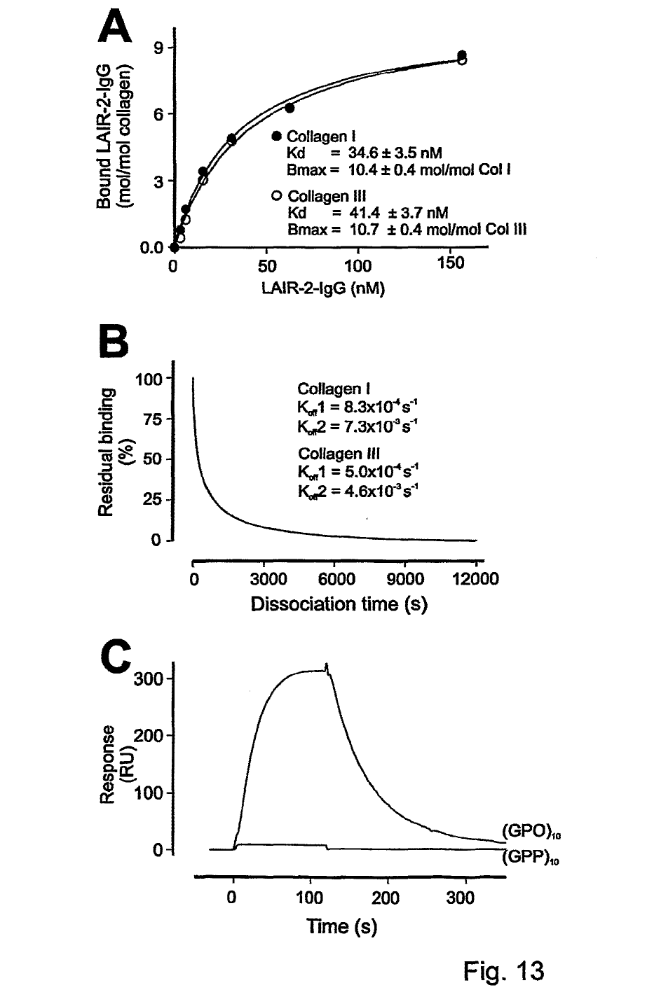

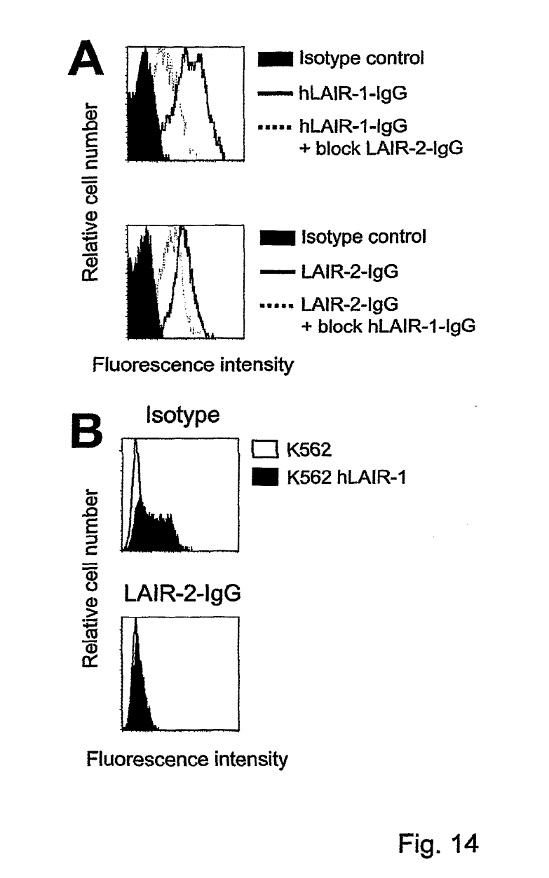

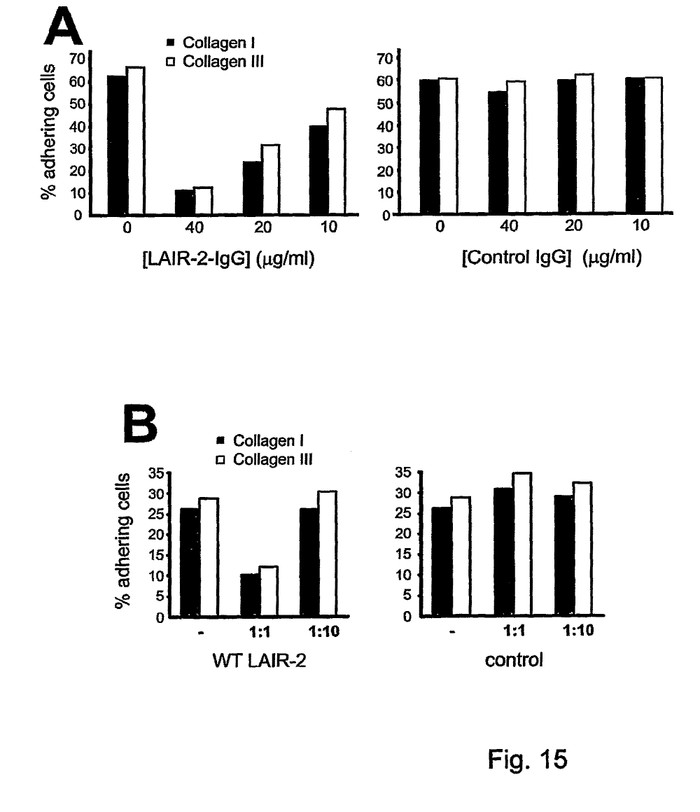

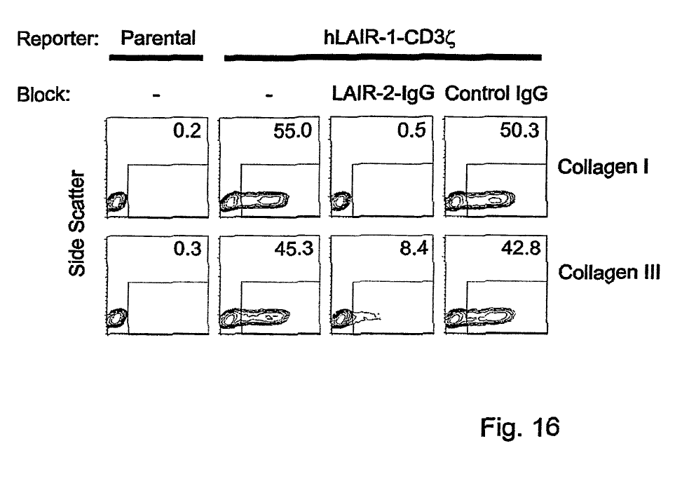

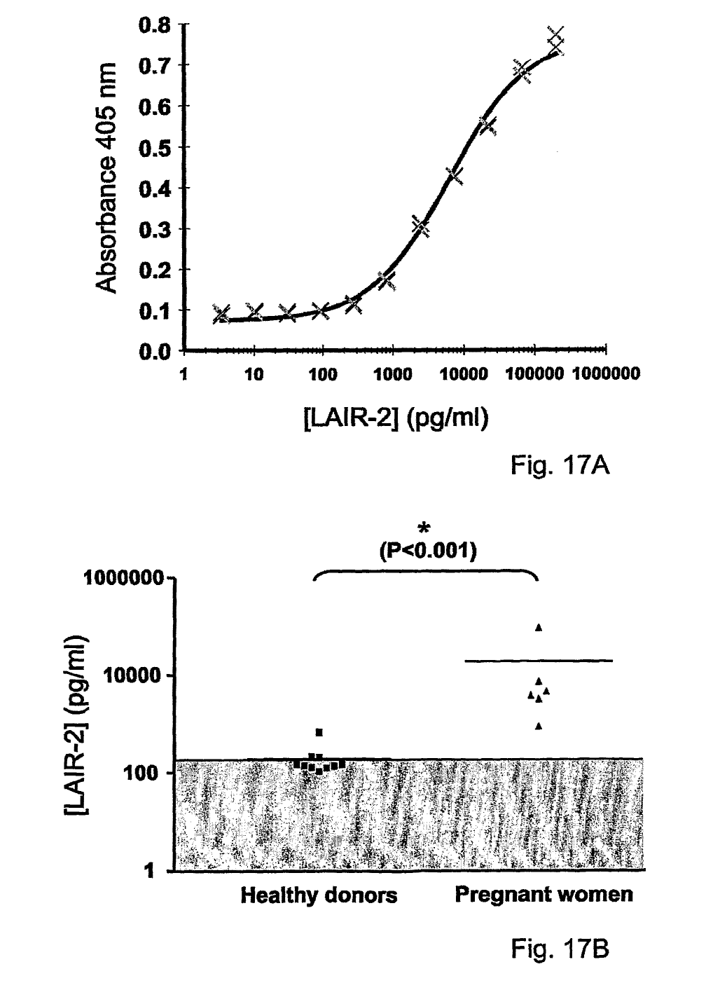

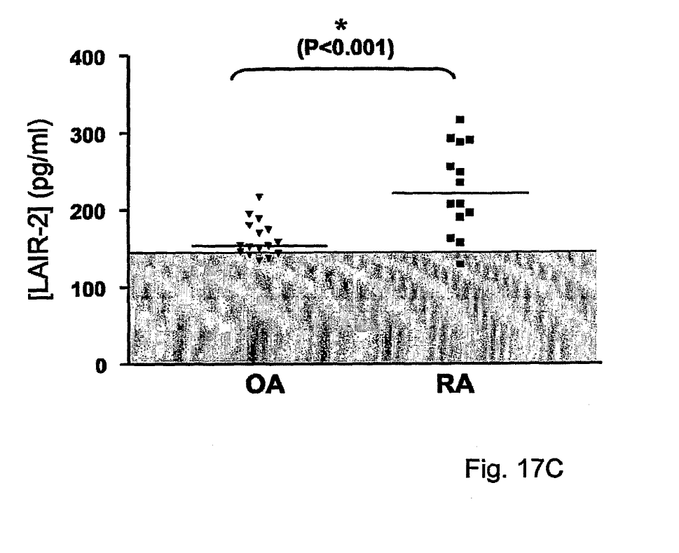

(A) The number of GPO triplets in the collagen II and III Toolkit peptides was correlated with the ability of the peptides to induce functional cross-linking of hLAIR-1-CD3ζ chimeric molecules on the surface of 2B4 NFAT-GFP reporter cells. Peptides that induced GFP-expression in >3% of the reporter cells were scored as positive. The percentage of responding peptides of the total amount of peptides containing a given number of GPO triplets is indicated. From the total 113 Toolkit peptides, 40 peptides contained 0 GPO triplets, 49 contained 1 GPO triplet, 19 contained 2 GPO triplets, 4 contained 3 GPO triplets, and 1 peptide contained 4 GPO triplets. The number of peptides that induced GFP expression out of the total number of peptides with a given number of GPO triplets is indicated (B) An increased GPO-content in Toolbox peptides results in increased inhibition of the degranulation response via hLAIR-1. Human LAIR-1 transfected (closed bars) or untransfected (open bars) RBL-2H3 cells were sensitized with IgE anti-TNP and subsequently incubated at 37° C. in plates coated with TNP conjugated to BSA (0.8 μg/ml) and synthetic trimeric peptides (1 μg/ml). The assay was identical as described in FIG. 4. The sequences of the indicated peptides are shown in supplemental table III. One representative experiment of two is shown. (C) An increased GPO-content in Toolbox peptides results in increased inhibition of CD3-induced T cell activation via hLAIR-1. Human LAIR-1 transfected NFAT-GFP reporter T cells were incubated with immobilized anti-CD3 mAbs and 10, 3.3, or 1.1 μg/ml immobilized synthetic trimeric peptides for 22 hours and GFP expression was analyzed by flow cytometry. The assay was identical as described in FIG. 3. Of note: this experiment was performed once.

FIG. 12. LAIR-2 is expressed as a soluble protein by various hematopoietic cell types.

A, RNA samples obtained from human PBMCs and various human cell lines were converted to cDNA by RT PCR. The cDNA fragments were amplified using LAIR-2 (top panel) or GAPDH (bottom panel) specific primers. RT PCR analysis was performed on RNA isolated from the following cells: peripheral blood mononuclear cells (PBMCs), T cell lines Jurkat and CEM, monocytic cell lines U937 and THP-1, NK-like YT.2C2 cells, B cell line 721.221, 293T embryonic kidney cells, colon carcinoma cell lines HT29, DLD-1, LS174, SW480 and HCT116, SKBR3 breast carcinoma cells and a water control (data not shown). B, Permeabilized 293T cells transiently transfected with cDNA encoding LAIR-2 (right panel) or control cDNA (left panel) were intracellularly stained using anti-LAIR-2 mAbs (filled histograms) or isotype controls (open histograms). Binding of anti-LAIR-2 mAbs was detected with PE-conjugated goat-anti-mouse IgG mAbs. C, Reducing Western blot analysis of supernatant from control cDNA transfected (Cont., left lane) and LAIR-2 transfected 293T cells (right lane) for presence of LAIR-2.

FIG. 13. LAIR-2 is a high affinity collagen receptor.

A, Binding of LAIR-2 to collagen I and III as indicated by surface plasmon resonance. LAIR-2-IgG concentrations were injected at 20 μl/min sequentially through a BIAcore flow cell containing ˜2000-3000 RU of immobilized collagen I (•) or III (∘). Each symbol represents the resonance at equilibrium and the corresponding concentration of the LAIR-2 fusion protein. Calculated affinities (Kd) and number of maximal binding sites (Bmax) are indicated. B, Rate of dissociation of LAIR-2-IgG from collagen III as determined by surface plasmon resonance. The dissociation curve of LAIR-2-IgG from collagen I was comparable (data not shown). Calculated dissociation values are indicated. C, LAIR-2 fusion protein was injected at 5 μl/min through a BIAcore flow cell containing ˜250 RU of immobilized (GPO)₁₀ or (GPP)₁₀ trimeric peptides.

FIG. 14. LAIR-2-IgG blocks the interaction of hLAIR-1 to endogenous collagens on HT29 cells and purified extracellular matrix collagen IV.

A, Human colon carcinoma HT29 cells were pre-incubated for 30 min with LAIR-2-IgG (top panel) or hLAIR-1-IgG (lower panel) and subsequently stained with biotin-conjugated human LAIR fusion proteins. Specific binding of the biotinylated fusion proteins was detected with APC conjugated streptavidin. B, Parental (open histograms) or human LAIR-1 transfected K562 cells (closed histograms) were incubated with Oregon green 488 labeled collagen IV which was either pre-incubated with 40 μg/ml LAIR-2-IgG (lower panel) or control Ig fusion proteins (isotype, top panel). Cells were analyzed by flow cytometry.

FIG. 15. LAIR-2 blocks the binding of hLAIR-1 to collagens I and III.

A, 96-well plates were coated with collagen I (gray bars) or collagen III (open bars). Subsequently the wells were incubated with different indicated concentrations of LAIR-2 fusion proteins (left panel) or control fusion proteins (right panel). Fluorescently labeled K562 cells expressing hLAIR-1 were allowed to interact for ˜3 hours. Percentage of adhering cells relative to the input signal is shown. Cells did not adhere to wells coated with BSA. B, Concentrated supernatants of 293T cells transfected with LAIR-2 (WT LAIR-2, left panel) or control cDNA (right panel), were analyzed for their capacity to block adhesion of hLAIR-1 transfected K562 cells to immobilized collagens I and III, as described above. The dilution of concentrated supernatants is indicated.

FIG. 16. LAIR-2 blocks the functional interaction between LAIR-1 and collagen I and III.

NFAT-GFP reporter cells (40) transfected with (right 3 panels) or without hLAIR-1-CD3ζ chimeric molecules (left panels) were incubated with immobilized collagen I (top panels) or III (lower panels) that were pre-incubated with or without LAIR-2-IgG or control fusion proteins. After approximately 20 hours, GFP expression was analyzed by flow cytometry. Percentage of GFP-positive cells is indicated in each plot.

FIG. 17. LAIR-2 is present in urine of pregnant women and in synovial fluid of RA

patients. A. Calibration curve of LAIR-2 ELISA. The plotted values were obtained with 3, 10, 30, 91, 274, 823, 2.5×10³, 7.4×10³, 2.2×10⁴, 6.7×10⁴ and 2.0×10⁵ pg/ml recombinant LAIR-2 proteins (R&D systems). Detection limit is 150 pg/ml. B, Presence of LAIR-2 in urine of non-pregnant donors (5 men and 5 women) and 6 pregnant women (16-37 weeks of gestation). Measurements were performed by ELISA as described in material and methods. A Mann-Whitney test indicated a statistical significant elevation of LAIR-2 levels in urine of pregnant women as compared to healthy controls (P<0.001). C, Presence of LAIR-2 in synovial fluid of patients with rheumatoid arthritis (RA) or osteoarthritis (OA). A Mann-Whitney test indicated a statistical significant elevation of LAIR-2 levels in synovial fluid of RA patients as compared to OA patients (P<0.001). Shaded areas indicate the detection limit in figure B and C.

### REFERENCE LIST (ALSO FOR EXAMPLES 1 AND 2)

1. Myllyharju, J. and K. I. Kivirikko. 2004. Collagens, modifying enzymes and their mutations in humans, flies and worms. Trends Genet. 20:33-43.
2. Gelse, K., E. Poschl, and T. Aigner. 2003. Collagens—structure, function, and biosynthesis. Adv. Drug Deliv. Rev. 55:1531-1546.
3. Meyaard, L., G. J. Adema, C. Chang, E. Woollatt, G. R. Sutherland, L. L. Lanier, and J. H. Phillips. 1997. LAIR-1, a novel inhibitory receptor expressed on human mononuclear leukocytes. Immunity. 7:283-290.
4. van der Vuurst de Vries A R, H. Clevers, T. Logtenberg, and L. Meyaard. 1999. Leukocyte-associated immunoglobulin-like receptor-1 (LAIR-1) is differentially expressed during human B cell differentiation and inhibits B cell receptor-mediated signaling. Eur. J. Immunol. 29:3160-3167.
5. Poggi, A., E. Tomasello, E. Ferrero, M. R. Zocchi, and L. Moretta. 1998. p40/LAIR-1 regulates the differentiation of peripheral blood precursors to dendritic cells induced by granulocyte-monocyte colony-stimulating factor. Eur. J. Immunol. 28:2086-2091.
6. Maasho, K., M. Masilamani, R. Valas, S. Basu, J. E. Coligan, and F. Borrego. 2005. The inhibitory leukocyte-associated Ig-like receptor-1 (LAIR-1) is expressed at high levels by human naive T cells and inhibits TCR mediated activation. Mol. Immunol. 42:1521-1530.
7. Verbrugge, A., T. de Ruiter, H. Clevers, and L. Meyaard. 2003. Differential contribution of the Immunoreceptor Tyrosine-based Inhibitory Motifs (ITIMs) of human Leukocyte Associated Ig-like Receptor (LAIR)-1 to inhibitory function and phosphatase recruitment. Int. Immunol. 15:1349-1358.
8. Ravetch, J. V. and L. L. Lanier. 2000. Immune inhibitory receptors. Science 290:84-89.
9. Pritchard, N. R. and K. G. Smith. 2003. B cell inhibitory receptors and autoimmunity. Immunology 108:263-273.
10. Meyaard, L., J. Hurenkamp, H. Clevers, L. L. Lanier, and J. H. Phillips. 1999. Leukocyte-associated Ig-like receptor-1 functions as an inhibitory receptor on cytotoxic T cells. J. Immunol. 162:5800-5804.
11. Lebbink, R. J., T. de Ruiter, A. Verbrugge, W. S. Bril, and L. Meyaard. 2004. The mouse homologue of the leukocyte-associated Ig-like receptor-1 is an inhibitory receptor that recruits Src homology region 2-containing protein tyrosine phosphatase (SHP)-2, but not SHP-1. J. Immunol. 172:5535-5543.
12. Lebbink, R. J., T. de Ruiter, G. J. Kaptijn, and L. Meyaard. 2005. Identification and characterization of the rat homologue of LAIR-1. Immunogenetics 57:344-351.
13. Myllyharju, J., K. I. Kivirikko. 2004. Collagens, modifying enzymes and their mutations in humans, flies and worms. Trends Genet. Vol. 20 No. 1: 33-43.
14. Maenaka, K., T. Juji, T. Nakayama, J. R. Wyer, G. F. Gao, T. Maenaka, N. R. Zaccai, A. Kikuchi, T. Yabe, K. Tokunaga, K. Tadokoro, D. I. Stuart, E. Y. Jones, and P. A. van der Merwe. 1999. Killer cell immunoglobulin receptors and T cell receptors bind peptide-major histocompatibility complex class I with distinct thermodynamic and kinetic properties. J. Biol. Chem. 274:28329-28334.
15. Ricard-Blum, S. and F. Ruggiero. 2005. The collagen superfamily: from the extracellular matrix to the cell membrane. Pathol. Biol. (Paris) 53:430-442.
16. Sixma, J. J., P. G. de Groot, H. van Zanten, and M. IJsseldijk. 1998. A new perfusion chamber to detect platelet adhesion using a small volume of blood. Thromb. Res. 92:S43-S46.
17. Farndale, R. W., J. J. Sixma, M. J. Barnes, and P. G. de Groot. 2004. The role of collagen in thrombosis and hemostasis. J. Thromb. Haemost. 2:561-573.
18. Verbrugge, A., T. de Ruiter, C. Geest, P. J. Coffer, and L. Meyaard. 2006. Differential expression of Leukocyte Associated Ig-like Receptor-1 during neutrophil differentiation and activation. J. Leukoc. Biol. In press.
19. Franzke, C. W., P. Bruckner, and L. Bruckner-Tuderman. 2005. Collagenous transmembrane proteins: recent insights into biology and pathology. J. Biol. Chem. 280:4005-4008.
20. Clague, R. B. and L. J. Moore. 1984. IgG and IgM antibody to native type II collagen in rheumatoid arthritis serum and synovial fluid. Evidence for the presence of collagen-anticollagen immune complexes in synovial fluid. Arthritis Rheum. 27:1370-1377.
21. Gioud, M., A. Meghlaoui, O. Costa, and J. C. Monier. 1982. Antibodies to native type I and II collagens detected by an enzyme linked immunosorbent assay (ELISA) in rheumatoid arthritis and systemic lupus erythematosus. Coll. Relat Res. 2:557-564.
22. Schmidt, E. and D. Zillikens. 2000. Autoimmune and inherited subepidermal blistering diseases: advances in the clinic and the laboratory. Adv. Dermatol. 16:113-157.
23. Lanier, L. L. 2005. NK Cell Recognition. Annu. Rev. Immunol. 225-274.
24. Parikka, M., T. Kainulainen, K. Tasanen, A. Vaananen, L. Bruckner-Tuderman, and T. Salo. 2003. Alterations of collagen XVII expression during transformation of oral epithelium to dysplasia and carcinoma. J. Histochem. Cytochem. 51:921-929.
25. Iizasa, T., H. Chang, M. Suzuki, M. Otsuji, S. Yokoi, M. Chiyo, S. Motohashi, K. Yasufuku, Y. Sekine, A. Iyoda, K. Shibuya, K. Hiroshima, and T. Fujisawa. 2004. Overexpression of collagen XVIII is associated with poor outcome and elevated levels of circulating serum endostatin in non-small cell lung cancer. Clin. Cancer Res. 10:5361-5366.
26. Banyard, J., L. Bao, and B. R. Zetter. 2003. Type XXIII collagen, a new transmembrane collagen identified in metastatic tumor cells. J. Biol. Chem. 278:20989-20994.
27. Roepman, P., L. F. Wessels, N. Kettelarij, P. Kemmeren, A. J. Miles, P. Lijnzaad, M. G. Tilanus, R. Koole, G. J. Hordijk, P. C. van der Vliet, M. J. Reinders, P. J. Slootweg, and F. C. Holstege. 2005. An expression profile for diagnosis of lymph node metastases from primary head and neck squamous cell carcinomas. Nat. Genet. 37:182-186.
28. Vaisanen, T., M. R. Vaisanen, H. Autio-Harmainen, and T. Pihlajaniemi, 2005. Type XIII collagen expression is induced during malignant transformation in various epithelial and mesenchymal tumours. J. Pathol. 207:324-335.
29. Naviaux, R. K., E. Costanzi, M. Haas, and I. M. Verma. 1996. The pCL vector system: rapid production of helper-free, high-titer, recombinant retroviruses. J. Virol. 70:5701-5705.
30. Meyaard, L., van der Vuurst de Vries, A., de Ruiter, T., Lanier, L. L., Phillips, J. H. and Clevers, H. 2001. The epithelial cellular adhesion molecule (Ep-CAM) is a ligand for the leukocyte-associated immunoglobulin-like receptor (LAIR). J. Exp. Med. 194:107-112.
31. Meyaard, L., van der Vuurst de Vries, A., de Ruiter, T., Lanier, L. L., Phillips, J. H. and Clevers, H. 2003. Retraction of: J. Exp. Med. 2001, 194: 107-112. J Exp Med. 198:1129
32. Voehringer, D., D. B. Rosen, L. L. Lanier, and R. M. Locksley. 2004. CD200 receptor family members represent novel DAP12-associated activating receptors on basophils and mast cells. J. Biol. Chem. 279:54117-54123.

### REFERENCE FOR SUPPLEMENTAL MATERIALS AND METHODS (EXAMPLE 1)

1. Naviaux, R. K., Costanzi, E., Haas, M. & Verma, I. M. The pCL vector system: rapid production of helper-free, high-titer, recombinant retroviruses. J. Virol. 70, 5701-5705 (1996).
2. Roepman, P. et al. An expression profile for diagnosis of lymph node metastases from primary head and neck squamous cell carcinomas. Nat. Genet. 37, 182-186 (2005).
3. Van de Peppel, P. J. et al. Monitoring global messenger RNA changes in externally controlled microarray experiments. EMBO Rep. 4, 387-393 (2003).
4. Tusher, V. G., Tibshirani, R. & Chu, G. Significance analysis of microarrays applied to the ionizing radiation response. Proc. Natl. Acad. Sci. U. S. A 98, 5116-5121 (2001).
5. Kinter, M. & Sherman N. E. (Wiley and Sons, New York,2000).
6. Knight, C. G. et al. Collagen-platelet interaction: Gly-Pro-Hyp is uniquely specific for platelet Gp VI and mediates platelet activation by collagen. Cardiovasc. Res. 41, 450-457 (1999).

1. A method for upregulating or downregulating activation of an immune cell, comprising providing a substance which specifically interacts in the binding of leukocyte-associated immunoglobulin-like receptor (LAIR) and collagen.

2. The method of claim 1, wherein the substance specifically interacts in the binding of LAIR and a G-X-Y repeat (wherein X and Y are amino acid residues) in collagen.

3. The method of claim 2, wherein said X is Proline, and wherein said Y is Hydroxyproline.

4. The method of claim 1, wherein said LAIR is LAIR-1.

5. The method of claim 1, which is a down regulation and the substance is a ligand for LAIR-1.

6. The method of claim 5, wherein the ligand for LAIR-1 is an anti-LAIR-1 antibody or a functional part, derivative and/or analogue thereof.

7. The method of claim 5, wherein the ligand for LAIR-1 comprises a peptide comprising several G-X-Y repeats.

8. The method of claim 7, wherein said G-X-Y repeats comprise several G-P-O repeats.

9. The method of claim 7, wherein said peptide forms a triple-helical peptide.

10. The method of claim 1, which is up regulation and the substance binds specifically to LAIR, and binds only one site.

11. The method of claim 1, which is up regulation and the substance binds specifically to collagen.

12. The method of claim 11, wherein the substance binds to a G-X-Y repeat.

13. The method of claim 11, wherein said substance is an antibody or a functional part, derivative and/or analogue thereof, against a collagen.

14. The method of claim 11, wherein said substance is a secreted LAIR or a functional part, derivative and/or analogue thereof.

15. The method of claim 11, wherein said collagen is collagen I, II, III, XIII, XVII, or XXIII.

16. A pharmaceutical composition comprising a peptide having G-X-Y repeats and a suitable carrier.

17. A pharmaceutical composition of claim 16, wherein said peptide forms a triple-helical peptide.

18. A pharmaceutical composition comprising a human or humanised antibody or a functional part, derivative and/or analogue thereof against collagen and a suitable carrier, or
comprising LAIR-2 or a functional part, derivative and/or analogue thereof and a suitable carrier.

19. (canceled)

20. A pharmaceutical composition of claim 16, wherein said peptide comprises a sequence with a length of between 15 and 50 amino acid residues, which sequence is at least 80% homologous to at least part of the sequence GPMGPMGPRGPOGPAGAOGPQGFQGNO (SEQ ID NO:1), GTOGTDGPKGASGPAGPOGAQGPOGLQ (SEQ ID NO:2), GRAGEOGLQGPAGPOGEKGEOGDDGPS (SEQ ID NO:3), GAOGAOGPOGSOGPAGPTGKQGDRGEA (SEQ ID NO:4), GPRGRSGETGPAGPOGNOGPOGPOGPO (SEQ ID NO:5), GLAGYOGPAGPOGPOGPOGTSGHOGSO (SEQ ID NO:6), GERGLOGPOGIKGPAGIOGFOGMKGHR (SEQ ID NO:7), GAOGLRGGAGPOGPEGGKGAAGPOGPO (SEQ ID NO:8), GMOGERGGLGSOGPKGDKGEOGGOGAD (SEQ ID NO:9), GEGGPOGVAGPOGGSGPAGPOGPQGVK (SEQ ID NO:10), GAOGPLGIAGITGARGLAGPOGMOGPR (SEQ ID NO:11), GPOGMOGPRGSOGPQGVKGESGKOGAN (SEQ ID NO:12) and/or GPAGPAGAOGPAGSRGAOGPQGPRGDK (SEQ ID NO:13), said part having at least 15 amino acid residues.

21. A pharmaceutical composition of claim 16, wherein said peptide comprises a sequence with a length of between 15 and 50 amino acid residues, which sequence is at least 80% homologous to at least part of the sequence GAOGLRGGAGPOGPEGGKGAAGPOGPO (SEQ ID NO:8), GPRGRSGETGPAGPOGNOGPOGPOGPO (SEQ ID NO:5), GTOGTDGPKGASGPAGPOGAQGPOGLQ (SEQ ID NO:2), GEGGPOGVAGPOGGSGPAGPOGPQGVK (SEQ ID NO:10), GRAGEOGLQGPAGPOGEKGEOGDDGPS (SEQ ID NO:3) and/or GPAGPAGAOGPAGSRGAOGPQGPRGDK (SEQ ID NO:13), said part having at least 15 amino acid residues.

22-31. (canceled)

32. A method to at least in part prevent, ameliorate and/or cure an infection and/or a tumor-related disease, comprising administering a secreted LAIR and/or a soluble LAIR, or a functional part, derivative and/or analogue thereof to a subject suffering from, or at risk of suffering from, an infection and/or a tumor-related disease.

33. A method to at least in part prevent, ameliorate and/or cure an auto-immune disease comprising administering a compound capable of decreasing the amount and/or activity of LAIR-2 to a subject suffering from, or at risk of suffering from, an auto-immune disease.

34. The method of claim 33, wherein said compound comprises an anti-LAIR-2 antibody or a functional part, derivative and/or analogue thereof.

35. A method for determining whether an immune response in an individual is upregulated, comprising measuring the amount of LAIR-2 is a sample of said individual and determining whether said amount is indicative for an upregulated immune response.


SEQUENCE LISTING (139 sequences)

SEQ ID NO: 1 - protein, 27 aa, Artificial
  synthetically constructed LAIR-binding peptide
GPMGPMGPRGPPGPAGAPGPQGFQGNP

SEQ ID NO: 2 - protein, 27 aa, Artificial
  synthetically constructed LAIR-binding peptide
GTPGTDGPKGASGPAGPPGAQGPPGLQ

SEQ ID NO: 3 - protein, 27 aa, Artificial
  synthetically constructed LAIR-binding peptide
GRAGEPGLQGPAGPPGEKGEPGDDGPS

SEQ ID NO: 4 - protein, 27 aa, Artificial
  synthetically constructed LAIR-binding peptide
GAPGAPGPPGSPGPAGPTGKQGDRGEA

SEQ ID NO: 5 - protein, 27 aa, Artificial
  synthetically constructed LAIR-binding peptide
GPRGRSGETGPAGPPGNPGPPGPPGPP

SEQ ID NO: 6 - protein, 27 aa, Artificial
  synthetically constructed LAIR-binding peptide
GLAGYPGPAGPPGPPGPPGTSGHPGSP

SEQ ID NO: 7 - protein, 27 aa, Artificial
  synthetically constructed LAIR-binding peptide
GERGLPGPPGIKGPAGIPGFPGMKGHR

SEQ ID NO: 8 - protein, 27 aa, Artificial
  synthetically constructed LAIR-binding peptide
GAPGLRGGAGPPGPEGGKGAAGPPGPP

SEQ ID NO: 9 - protein, 27 aa, Artificial
  synthetically constructed LAIR-binding peptide
GMPGERGGLGSPGPKGDKGEPGGPGAD

SEQ ID NO: 10 - protein, 27 aa, Artificial
  synthetically constructed LAIR-binding peptide
GEGGPPGVAGPPGGSGPAGPPGPQGVK

SEQ ID NO: 11 - protein, 27 aa, Artificial
  synthetically constructed LAIR-binding peptide
GAPGPLGIAGITGARGLAGPPGMPGPR

SEQ ID NO: 12 - protein, 27 aa, Artificial
  synthetically constructed LAIR-binding peptide
GPPGMPGPRGSPGPQGVKGESGKPGAN

SEQ ID NO: 13 - protein, 27 aa, Artificial
  synthetically constructed LAIR-binding peptide
GPAGPAGAPGPAGSRGAPGPQGPRGDK

SEQ ID NO: 14 - protein, 11 aa, Artificial
  synthetically constructed LAIR-ligand
DGFTGDLDYNK

SEQ ID NO: 15 - protein, 37 aa, Artificial
  collagen related peptide
GCPGPPGPPGPPGPPGPPGPPGPPGPPGPPGPPGCPG

SEQ ID NO: 16 - protein, 37 aa, Artificial; identical to SEQ ID NO: 15
  synthetically constructed triple-helical peptide

SEQ ID NO: 17 - DNA, 20 bp, Artificial
  primer
gttggggttcaaacattccg

SEQ ID NO: 18 - DNA, 20 bp, Artificial
  primer
tcatggtgcatcaaatccgg

SEQ ID NO: 19 - DNA, 20 bp, Artificial
  primer
ggtacatgacaaggtgcggc

SEQ ID NO: 20 - DNA, 21 bp, Artificial
  primer
gcatcctgggctacactgagc

SEQ ID NO: 21 - protein, 63 aa, Artificial
  synthetically constructed peptide of collagen II toolkit
GPCGPPGPPGPPGPPGPPGPMGPMGPRGPPGPAGAPGPQGFQGNPGPPGPPGPPGPPGPPGPC

SEQ ID NO: 22 - protein, 63 aa, Artificial
  synthetically constructed peptide of collagen II toolkit
GPCGPPGPPGPPGPPGPPGPQGFQGNPGEPGEPGVSGPMGPRGPPGPPGPPGPPGPPGPPGPC

SEQ ID NO: 23 - protein, 63 aa, Artificial
  synthetically constructed peptide of collagen II toolkit
GPCGPPGPPGPPGPPGPPGPMGPRGPPGPPGKPGDDGEAGKPGKAGPPGPPGPPGPPGPPGPC

SEQ ID NO: 24 - protein, 63 aa, Artificial
  synthetically constructed peptide of collagen II toolkit
GPCGPPGPPGPPGPPGPPGEAGKPGKAGERGPPGPQGARGFPGTPGPPGPPGPPGPPGPPGPC

SEQ ID NO: 25 - protein, 63 aa, Artificial
  synthetically constructed peptide of collagen II toolkit
GPCGPPGPPGPPGPPGPPGARGFPGTPGLPGVKGHRGYPGLDGAKGPPGPPGPPGPPGPPGPC

SEQ ID NO: 26 - protein, 63 aa, Artificial
  synthetically constructed peptide of collagen II toolkit
GPCGPPGPPGPPGPPGPPGYPGLDGAKGEAGAPGVKGESGSPGENGPPGPPGPPGPPGPPGPC

SEQ ID NO: 27 - protein, 63 aa, Artificial
  synthetically constructed peptide of collagen II toolkit
GPCGPPGPPGPPGPPGPPGESGSPGENGSPGPMGPRGLPGERGRTGPPGPPGPPGPPGPPGPC

SEQ ID NO: 28 - protein, 63 aa, Artificial
  synthetically constructed peptide of collagen II toolkit
GPCGPPGPPGPPGPPGPPGLPGERGRTGPAGAAGARGNDGQPGPAGPPGPPGPPGPPGPPGPC

SEQ ID NO: 29 - protein, 63 aa, Artificial
  synthetically constructed peptide of collagen II toolkit
GPCGPPGPPGPPGPPGPPGNDGQPGPAGPPGPVGPAGGPGFPGAPGPPGPPGPPGPPGPPGPC

SEQ ID NO: 30 - protein, 63 aa, Artificial
  synthetically constructed peptide of collagen II toolkit
GPCGPPGPPGPPGPPGPPGGPGFPGAPGAKGEAGPTGARGPEGAQGPPGPPGPPGPPGPPGPC

SEQ ID NO: 31 - protein, 63 aa, Artificial
  synthetically constructed peptide of collagen II toolkit
GPCGPPGPPGPPGPPGPPGARGPEGAQGPRGEPGTPGSPGPAGASGPPGPPGPPGPPGPPGPC

SEQ ID NO: 32 - protein, 63 aa, Artificial
  synthetically constructed peptide of collagen II toolkit
GPCGPPGPPGPPGPPGPPGSPGPAGASGNPGTDGIPGAKGSAGAPGPPGPPGPPGPPGPPGPC

SEQ ID NO: 33 - protein, 63 aa, Artificial
  synthetically constructed peptide of collagen II toolkit
GPCGPPGPPGPPGPPGPPGAKGSAGAPGIAGAPGFPGPRGPPGPQGPPGPPGPPGPPGPPGPC

SEQ ID NO: 34 - protein, 63 aa, Artificial
  synthetically constructed peptide of collagen II toolkit
GPCGPPGPPGPPGPPGPPGPRGPPGPQGATGPLGPKGQTGEPGIAGPPGPPGPPGPPGPPGPC

SEQ ID NO: 35 - protein, 63 aa, Artificial
  synthetically constructed peptide of collagen II toolkit
GPCGPPGPPGPPGPPGPPGQTGEPGIAGFKGEQGPKGEPGPAGPQGPPGPPGPPGPPGPPGPC

SEQ ID NO: 36 - protein, 63 aa, Artificial
  synthetically constructed peptide of collagen II toolkit
GPCGPPGPPGPPGPPGPPGEPGPAGPQGAPGPAGEEGKRGARGEPGPPGPPGPPGPPGPPGPC

SEQ ID NO: 37 - protein, 63 aa, Artificial
  synthetically constructed peptide of collagen II toolkit
GPCGPPGPPGPPGPPGPPGKRGARGEPGGVGPIGPPGERGAPGNRGPPGPPGPPGPPGPPGPC

SEQ ID NO: 38 - protein, 63 aa, Artificial
  synthetically constructed peptide of collagen II toolkit
GPCGPPGPPGPPGPPGPPGERGAPGNRGFPGQDGLAGPKGAPGERGPPGPPGPPGPPGPPGPC

SEQ ID NO: 39 - protein, 63 aa, Artificial
  synthetically constructed peptide of collagen II toolkit
GPCGPPGPPGPPGPPGPPGPKGAPGERGPSGLAGPKGANGDPGRPGPPGPPGPPGPPGPPGPC

SEQ ID NO: 40 - protein, 63 aa, Artificial
  synthetically constructed peptide of collagen II toolkit
GPCGPPGPPGPPGPPGPPGANGDPGRPGEPGLPGARGLTGRPGDAGPPGPPGPPGPPGPPGPC

SEQ ID NO: 41 - protein, 63 aa, Artificial
  synthetically constructed peptide of collagen II toolkit
GPCGPPGPPGPPGPPGPPGLTGRPGDAGPQGKVGPSGAPGEDGRPGPPGPPGPPGPPGPPGPC

SEQ ID NO: 42 - protein, 63 aa, Artificial
  synthetically constructed peptide of collagen II toolkit
GPCGPPGPPGPPGPPGPPGAPGEDGRPGPPGPQGARGQPGVMGFPGPPGPPGPPGPPGPPGPC

SEQ ID NO: 43 - protein, 63 aa, Artificial
  synthetically constructed peptide of collagen II toolkit
GPCGPPGPPGPPGPPGPPGQPGVMGFPGPKGANGEPGKAGEKGLPGPPGPPGPPGPPGPPGPC

SEQ ID NO: 44 - protein, 63 aa, Artificial
  synthetically constructed peptide of collagen II toolkit
GPCGPPGPPGPPGPPGPPGKAGEKGLPGAPGLRGLPGKDGETGAAGPPGPPGPPGPPGPPGPC

SEQ ID NO: 45 - protein, 63 aa, Artificial
  synthetically constructed peptide of collagen II toolkit
GPCGPPGPPGPPGPPGPPGKDGETGAAGPPGPAGPAGERGEQGAPGPPGPPGPPGPPGPPGPC

SEQ ID NO: 46 - protein, 63 aa, Artificial
  synthetically constructed peptide of collagen II toolkit
GPCGPPGPPGPPGPPGPPGERGEQGAPGPSGFQGLPGPPGPPGEGGPPGPPGPPGPPGPPGPC

SEQ ID NO: 47 - protein, 63 aa, Artificial
  synthetically constructed peptide of collagen II toolkit
GPCGPPGPPGPPGPPGPPGPPGPPGEGGKPGDQGVPGEAGAPGLVGPPGPPGPPGPPGPPGPC

SEQ ID NO: 48 - protein, 63 aa, Artificial
  synthetically constructed peptide of collagen II toolkit
GPCGPPGPPGPPGPPGPPGEAGAPGLVGPRGERGFPGERGSPGAQGPPGPPGPPGPPGPPGPC

SEQ ID NO: 49 - protein, 63 aa, Artificial
  synthetically constructed peptide of collagen II toolkit
GPCGPPGPPGPPGPPGPPGTPGTDGPKGASGPAGPPGAQGPPGLQGPPGPPGPPGPPGPPGPC

SEQ ID NO: 50 - protein, 63 aa, Artificial
  synthetically constructed peptide of collagen II toolkit
GPCGPPGPPGPPGPPGPPGERGSPGAQGLQGPRGLPGTPGTDGPKGPPGPPGPPGPPGPPGPC

SEQ ID NO: 51 - protein, 63 aa, Artificial
  synthetically constructed peptide of collagen II toolkit
GPCGPPGPPGPPGPPGPPGAQGPPGLQGMPGERGAAGIAGPKGDRGPPGPPGPPGPPGPPGPC

SEQ ID NO: 52 - protein, 63 aa, Artificial
  synthetically constructed peptide of collagen II toolkit
GPCGPPGPPGPPGPPGPPGIAGPKGDRGDVGEKGPEGAPGKDGGRGPPGPPGPPGPPGPPGPC

SEQ ID NO: 53 - protein, 63 aa, Artificial
  synthetically constructed peptide of collagen II toolkit
GPCGPPGPPGPPGPPGPPGAPGKDGGRGLTGPIGPPGPAGANGEKGPPGPPGPPGPPGPPGPC

SEQ ID NO: 54 - protein, 63 aa, Artificial
  synthetically constructed peptide of collagen II toolkit
GPCGPPGPPGPPGPPGPPGPAGANGEKGEVGPPGPAGSAGARGAPGPPGPPGPPGPPGPPGPC

SEQ ID NO: 55 - protein, 63 aa, Artificial
  synthetically constructed peptide of collagen II toolkit
GPCGPPGPPGPPGPPGPPGSAGARGAPGERGETGPPGPAGFAGPPGPPGPPGPPGPPGPPGPC

SEQ ID NO: 56 - protein, 63 aa, Artificial
  synthetically constructed peptide of collagen II toolkit
GPCGPPGPPGPPGPPGPPGPAGFAGPPGADGQPGAKGEQGEAGQKGPPGPPGPPGPPGPPGPC

SEQ ID NO: 57 - protein, 63 aa, Artificial
  synthetically constructed peptide of collagen II toolkit
GPCGPPGPPGPPGPPGPPGEQGEAGQKGDAGAPGPQGPSGAPGPQGPPGPPGPPGPPGPPGPC

SEQ ID NO: 58 - protein, 63 aa, Artificial
  synthetically constructed peptide of collagen II toolkit
GPCGPPGPPGPPGPPGPPGPSGAPGPQGPTGVTGPKGARGAQGPPGPPGPPGPPGPPGPPGPC

SEQ ID NO: 59 - protein, 63 aa, Artificial
  synthetically constructed peptide of collagen II toolkit
GPCGPPGPPGPPGPPGPPGARGAQGPPGATGFPGAAGRVGPPGSNGPPGPPGPPGPPGPPGPC

SEQ ID NO: 60 - protein, 63 aa, Artificial
  synthetically constructed peptide of collagen II toolkit
GPCGPPGPPGPPGPPGPPGRVGPPGSNGNPGPPGPPGPSGKDGPKGPPGPPGPPGPPGPPGPC

SEQ ID NO: 61 - protein, 63 aa, Artificial
  synthetically constructed peptide of collagen II toolkit
GPCGPPGPPGPPGPPGPPGPSGKDGPKGARGDSGPPGRAGEPGLQGPPGPPGPPGPPGPPGPC

SEQ ID NO: 62 - protein, 63 aa, Artificial
  synthetically constructed peptide of collagen II toolkit
GPCGPPGPPGPPGPPGPPGRAGEPGLQGPAGPPGEKGEPGDDGPSGPPGPPGPPGPPGPPGPC

SEQ ID NO: 63 - protein, 63 aa, Artificial
  synthetically constructed peptide of collagen II toolkit
GPCGPPGPPGPPGPPGPPGEPGDDGPSGAEGPPGPQGLAGQRGIVGPPGPPGPPGPPGPPGPC

SEQ ID NO: 64 - protein, 63 aa, Artificial
  synthetically constructed peptide of collagen II toolkit
GPCGPPGPPGPPGPPGPPGLAGQRGIVGLPGQRGERGFPGLPGPSGPPGPPGPPGPPGPPGPC

SEQ ID NO: 65 - protein, 63 aa, Artificial
  synthetically constructed peptide of collagen II toolkit
GPCGPPGPPGPPGPPGPPGFPGLPGPSGEPGKQGAPGASGDRGPPGPPGPPGPPGPPGPPGPC

SEQ ID NO: 66 - protein, 63 aa, Artificial
  synthetically constructed peptide of collagen II toolkit
GPCGPPGPPGPPGPPGPPGASGDRGPPGPVGPPGLTGPAGEPGREGPPGPPGPPGPPGPPGPC

SEQ ID NO: 67 - protein, 63 aa, Artificial
  synthetically constructed peptide of collagen II toolkit
GPCGPPGPPGPPGPPGPPGPAGEPGREGSPGADGPPGRDGAAGVKGPPGPPGPPGPPGPPGPC

SEQ ID NO: 68 - protein, 63 aa, Artificial
  synthetically constructed peptide of collagen II toolkit
GPCGPPGPPGPPGPPGPPGRDGAAGVKGDRGETGAVGAPGAPGPPGPPGPPGPPGPPGPPGPC

SEQ ID NO: 69 - protein, 63 aa, Artificial
  synthetically constructed peptide of collagen II toolkit
GPCGPPGPPGPPGPPGPPGAPGAPGPPGSPGPAGPTGKQGDRGEAGPPGPPGPPGPPGPPGPC

SEQ ID NO: 70 - protein, 63 aa, Artificial
  synthetically constructed peptide of collagen II toolkit
GPCGPPGPPGPPGPPGPPGKQGDRGEAGAQGPMGPSGPAGARGIQGPPGPPGPPGPPGPPGPC

SEQ ID NO: 71 - protein, 63 aa, Artificial
  synthetically constructed peptide of collagen II toolkit
GPCGPPGPPGPPGPPGPPGPAGARGIQGPQGPRGDKGEAGEPGERGPPGPPGPPGPPGPPGPC

SEQ ID NO: 72 - protein, 63 aa, Artificial
  synthetically constructed peptide of collagen II toolkit
GPCGPPGPPGPPGPPGPPGEAGEPGERGLKGHRGFTGLQGLPGPPGPPGPPGPPGPPGPPGPC

SEQ ID NO: 73 - protein, 63 aa, Artificial
  synthetically constructed peptide of collagen II toolkit
GPCGPPGPPGPPGPPGPPGLQGLPGPPGPSGDQGASGPAGPSGPRGPPGPPGPPGPPGPPGPC

SEQ ID NO: 74 - protein, 63 aa, Artificial
  synthetically constructed peptide of collagen II toolkit
GPCGPPGPPGPPGPPGPPGPAGPSGPRGPPGPVGPSGKDGANGIPGPPGPPGPPGPPGPPGPC

SEQ ID NO: 75 - protein, 63 aa, Artificial
  synthetically constructed peptide of collagen II toolkit
GPCGPPGPPGPPGPPGPPGKDGANGIPGPIGPPGPRGRSGETGPAGPPGPPGPPGPPGPPGPC

SEQ ID NO: 76 - protein, 63 aa, Artificial
  synthetically constructed peptide of collagen II toolkit
GPCGPPGPPGPPGPPGPPGPRGRSGETGPAGPPGNPGPPGPPGPPGPPGPPGPPGPPGPPGPC

SEQ ID NO: 77 - protein, 63 aa, Artificial
  synthetically constructed peptide of collagen III toolkit
GPCGPPGPPGPPGPPGPPGLAGYPGPAGPPGPPGPPGTSGHPGSPGPPGPPGPPGPPGPPGPC

SEQ ID NO: 78 - protein, 63 aa, Artificial
  synthetically constructed peptide of collagen III toolkit
GPCGPPGPPGPPGPPGPPGTSGHPGSPGSPGYQGPPGEPGQAGPSGPPGPPGPPGPPGPPGPC

SEQ ID NO: 79 - protein, 63 aa, Artificial
  synthetically constructed peptide of collagen III toolkit
GPCGPPGPPGPPGPPGPPGEPGQAGPSGPPGPPGAIGPSGPAGKDGPPGPPGPPGPPGPPGPC

SEQ ID NO: 80 - protein, 63 aa, Artificial
  synthetically constructed peptide of collagen III toolkit
GPCGPPGPPGPPGPPGPPGPSGPAGKDGESGRPGRPGERGLPGPPGPPGPPGPPGPPGPPGPC

SEQ ID NO: 81 - protein, 63 aa, Artificial
  synthetically constructed peptide of collagen III toolkit
GPCGPPGPPGPPGPPGPPGERGLPGPPGIKGPAGIPGFPGMKGHRGPPGPPGPPGPPGPPGPC

SEQ ID NO: 82 - protein, 63 aa, Artificial
  synthetically constructed peptide of collagen III toolkit
GPCGPPGPPGPPGPPGPPGFPGMKGHRGFDGRNGEKGETGAPGLKGPPGPPGPPGPPGPPGPC

SEQ ID NO: 83 - protein, 63 aa, Artificial
  synthetically constructed peptide of collagen III toolkit
GPCGPPGPPGPPGPPGPPGETGAPGLKGENGLPGENGAPGPMGPRGPPGPPGPPGPPGPPGPC

SEQ ID NO: 84 - protein, 63 aa, Artificial
  synthetically constructed peptide of collagen III toolkit
GPCGPPGPPGPPGPPGPPGAPGPMGPRGAPGERGRPGLPGAAGARGPPGPPGPPGPPGPPGPC

SEQ ID NO: 85 - protein, 63 aa, Artificial
  synthetically constructed peptide of collagen III toolkit
GPCGPPGPPGPPGPPGPPGLPGAAGARGNDGARGSDGQPGPPGPPGPPGPPGPPGPPGPPGPC

SEQ ID NO: 86 - protein, 63 aa, Artificial
  synthetically constructed peptide of collagen III toolkit
GPCGPPGPPGPPGPPGPPGQPGPPGPPGTAGFPGSPGAKGEVGPAGPPGPPGPPGPPGPPGPC

SEQ ID NO: 87 - protein, 63 aa, Artificial
  synthetically constructed peptide of collagen III toolkit
GPCGPPGPPGPPGPPGPPGAKGEVGPAGSPGSNGAPGQRGEPGPQGPPGPPGPPGPPGPPGPC

SEQ ID NO: 88 - protein, 63 aa, Artificial
  synthetically constructed peptide of collagen III toolkit
GPCGPPGPPGPPGPPGPPGQRGEPGPQGHAGAQGPPGPPGINGSPGPPGPPGPPGPPGPPGPC

SEQ ID NO: 89 - protein, 63 aa, Artificial
  synthetically constructed peptide of collagen III toolkit
GPCGPPGPPGPPGPPGPPGPPGINGSPGGKGEMGPAGIPGAPGLMGPPGPPGPPGPPGPPGPC

SEQ ID NO: 90 - protein, 63 aa, Artificial
  synthetically constructed peptide of collagen III toolkit
GPCGPPGPPGPPGPPGPPGIPGAPGLMGARGPPGPAGANGAPGLRGPPGPPGPPGPPGPPGPC

SEQ ID NO: 91 - protein, 63 aa, Artificial
  synthetically constructed peptide of collagen III toolkit
GPCGPPGPPGPPGPPGPPGANGAPGLRGGAGEPGKNGAKGEPGPRGPPGPPGPPGPPGPPGPC

SEQ ID NO: 92 - protein, 63 aa, Artificial
  synthetically constructed peptide of collagen III toolkit
GPCGPPGPPGPPGPPGPPGAKGEPGPRGERGEAGIPGVPGAKGEDGPPGPPGPPGPPGPPGPC

SEQ ID NO: 93 - protein, 63 aa, Artificial
  synthetically constructed peptide of collagen III toolkit
GPCGPPGPPGPPGPPGPPGVPGAKGEDGKDGSPGEPGANGLPGAAGPPGPPGPPGPPGPPGPC

SEQ ID NO: 94 - protein, 63 aa, Artificial
  synthetically constructed peptide of collagen III toolkit
GPCGPPGPPGPPGPPGPPGANGLPGAAGERGAPGFRGPAGPNGIPGPPGPPGPPGPPGPPGPC

SEQ ID NO: 95 - protein, 63 aa, Artificial
  synthetically constructed peptide of collagen III toolkit
GPCGPPGPPGPPGPPGPPGPAGPNGIPGEKGPAGERGAPGPAGPRGPPGPPGPPGPPGPPGPC

SEQ ID NO: 96 - protein, 63 aa, Artificial
  synthetically constructed peptide of collagen III toolkit
GPCGPPGPPGPPGPPGPPGAPGPAGPRGAAGEPGRDGVPGGPGMRGPPGPPGPPGPPGPPGPC

SEQ ID NO: 97 - protein, 63 aa, Artificial
  synthetically constructed peptide of collagen III toolkit
GPCGPPGPPGPPGPPGPPGVPGGPGMRGMPGSPGGPGSDGKPGPPGPPGPPGPPGPPGPPGPC

SEQ ID NO: 98 - protein, 63 aa, Artificial
  synthetically constructed peptide of collagen III toolkit
GPCGPPGPPGPPGPPGPPGSDGKPGPPGSQGESGRPGPPGPSGPRGPPGPPGPPGPPGPPGPC

SEQ ID NO: 99 - protein, 63 aa, Artificial
  synthetically constructed peptide of collagen III toolkit
GPCGPPGPPGPPGPPGPPGPPGPSGPRGQPGVMGFPGPKGNDGAPGPPGPPGPPGPPGPPGPC

SEQ ID NO: 100 - protein, 63 aa, Artificial
  synthetically constructed peptide of collagen III toolkit
GPCGPPGPPGPPGPPGPPGPKGNDGAPGKNGERGGPGGPGPQGPPGPPGPPGPPGPPGPPGPC

SEQ ID NO: 101 - protein, 63 aa, Artificial
  synthetically constructed peptide of collagen III toolkit
GPCGPPGPPGPPGPPGPPGGPGPQGPPGKNGETGPQGPPGPTGPGGPPGPPGPPGPPGPPGPC

SEQ ID NO: 102 - protein, 63 aa, Artificial
  synthetically constructed peptide of collagen III toolkit
GPCGPPGPPGPPGPPGPPGPPGPTGPGGDKGDTGPPGPQGLQGLPGPPGPPGPPGPPGPPGPC

SEQ ID NO: 103 - protein, 63 aa, Artificial
  synthetically constructed peptide of collagen III toolkit
GPCGPPGPPGPPGPPGPPGPQGLQGLPGTGGPPGENGKPGEPGPKGPPGPPGPPGPPGPPGPC

SEQ ID NO: 104 - protein, 63 aa, Artificial
  synthetically constructed peptide of collagen III toolkit
GPCGPPGPPGPPGPPGPPGKPGEPGPKGDAGAPGAPGGKGDAGAPGPPGPPGPPGPPGPPGPC

SEQ ID NO: 105 - protein, 63 aa, Artificial
  synthetically constructed peptide of collagen III toolkit
GPCGPPGPPGPPGPPGPPGGKGDAGAPGERGPPGLAGAPGLRGGAGPPGPPGPPGPPGPPGPC

SEQ ID NO: 106 - protein, 63 aa, Artificial
  synthetically constructed peptide of collagen III toolkit
GPCGPPGPPGPPGPPGPPGAPGLRGGAGPPGPEGGKGAAGPPGPPGPPGPPGPPGPPGPPGPC

SEQ ID NO: 107 - protein, 63 aa, Artificial
  synthetically constructed peptide of collagen III toolkit
GPCGPPGPPGPPGPPGPPGAAGPPGPPGAAGTPGLQGMPGERGGLGPPGPPGPPGPPGPPGPC

SEQ ID NO: 108 - protein, 63 aa, Artificial
  synthetically constructed peptide of collagen III toolkit
GPCGPPGPPGPPGPPGPPGMPGERGGLGSPGPKGDKGEPGGPGADGPPGPPGPPGPPGPPGPC

SEQ ID NO: 109 - protein, 63 aa, Artificial
  synthetically constructed peptide of collagen III toolkit
GPCGPPGPPGPPGPPGPPGEPGGPGADGVPGKDGPRGPTGPIGPPGPPGPPGPPGPPGPPGPC

SEQ ID NO: 110 - protein, 63 aa, Artificial
  synthetically constructed peptide of collagen III toolkit
GPCGPPGPPGPPGPPGPPGPTGPIGPPGPAGQPGDKGEGGAPGLPGPPGPPGPPGPPGPPGPC

SEQ ID NO: 111 - protein, 63 aa, Artificial
  synthetically constructed peptide of collagen III toolkit
GPCGPPGPPGPPGPPGPPGEGGAPGLPGIAGPRGSPGERGETGPPGPPGPPGPPGPPGPPGPC

SEQ ID NO: 112 - protein, 63 aa, Artificial
  synthetically constructed peptide of collagen III toolkit
GPCGPPGPPGPPGPPGPPGERGETGPPGPAGFPGAPGQNGEPGGKGPPGPPGPPGPPGPPGPC

SEQ ID NO: 113 - protein, 63 aa, Artificial
  synthetically constructed peptide of collagen III toolkit
GPCGPPGPPGPPGPPGPPGQNGEPGGKGERGAPGEKGEGGPPGVAGPPGPPGPPGPPGPPGPC

SEQ ID NO: 114 - protein, 63 aa, Artificial
  synthetically constructed peptide of collagen III toolkit
GPCGPPGPPGPPGPPGPPGEGGPPGVAGPPGGSGPAGPPGPQGVKGPPGPPGPPGPPGPPGPC

SEQ ID NO: 115 - protein, 63 aa, Artificial
  synthetically constructed peptide of collagen III toolkit
GPCGPPGPPGPPGPPGPPGPPGPQGVKGERGSPGGPGAAGFPGARGPPGPPGPPGPPGPPGPC

SEQ ID NO: 116 - protein, 63 aa, Artificial
  synthetically constructed peptide of collagen III toolkit
GPCGPPGPPGPPGPPGPPGAAGFPGARGLPGPPGSNGNPGPPGPSGPPGPPGPPGPPGPPGPC

SEQ ID NO: 117 - protein, 63 aa, Artificial
  synthetically constructed peptide of collagen III toolkit
GPCGPPGPPGPPGPPGPPGNPGPPGPSGSPGKDGPPGPAGNTGAPGPPGPPGPPGPPGPPGPC

SEQ ID NO: 118 - protein, 63 aa, Artificial
  synthetically constructed peptide of collagen III toolkit
GPCGPPGPPGPPGPPGPPGPAGNTGAPGSPGVSGPKGDAGQPGEKGPPGPPGPPGPPGPPGPC

SEQ ID NO: 119 - protein, 63 aa, Artificial
  synthetically constructed peptide of collagen III toolkit
GPCGPPGPPGPPGPPGPPGDAGQPGEKGSPGAQGPPGAPGPLGIAGPPGPPGPPGPPGPPGPC

SEQ ID NO: 120 - protein, 63 aa, Artificial
  synthetically constructed peptide of collagen III toolkit
GPCGPPGPPGPPGPPGPPGAPGPLGIAGITGARGLAGPPGMPGPRGPPGPPGPPGPPGPPGPC

SEQ ID NO: 121 - protein, 63 aa, Artificial
  synthetically constructed peptide of collagen III toolkit
GPCGPPGPPGPPGPPGPPGPPGMPGPRGSPGPQGVKGESGKPGANGPPGPPGPPGPPGPPGPC

SEQ ID NO: 122 - protein, 63 aa, Artificial
  synthetically constructed peptide of collagen III toolkit
GPCGPPGPPGPPGPPGPPGESGKPGANGLSGERGPPGPQGLPGLAGPPGPPGPPGPPGPPGPC

SEQ ID NO: 123 - protein, 63 aa, Artificial
  synthetically constructed peptide of collagen III toolkit
GPCGPPGPPGPPGPPGPPGPQGLPGLAGTAGEPGRDGNPGSDGLPGPPGPPGPPGPPGPPGPC

SEQ ID NO: 124 - protein, 63 aa, Artificial
  synthetically constructed peptide of collagen III toolkit
GPCGPPGPPGPPGPPGPPGNPGSDGLPGRDGSPGGKGDRGENGSPGPPGPPGPPGPPGPPGPC

SEQ ID NO: 125 - protein, 63 aa, Artificial
  synthetically constructed peptide of collagen III toolkit
GPCGPPGPPGPPGPPGPPGDRGENGSPGAPGAPGHPGPPGPVGPAGPPGPPGPPGPPGPPGPC

SEQ ID NO: 126 - protein, 63 aa, Artificial
  synthetically constructed peptide of collagen III toolkit
GPCGPPGPPGPPGPPGPPGPPGPVGPAGKSGDRGESGPAGPAGAPGPPGPPGPPGPPGPPGPC

SEQ ID NO: 127 - protein, 63 aa, Artificial
  synthetically constructed peptide of collagen III toolkit
GPCGPPGPPGPPGPPGPPGPAGPAGAPGPAGSRGAPGPQGPRGDKGPPGPPGPPGPPGPPGPC

SEQ ID NO: 128 - protein, 63 aa, Artificial
  synthetically constructed peptide of collagen III toolkit
GPCGPPGPPGPPGPPGPPGPQGPRGDKGETGERGAAGIKGHRGFPGPPGPPGPPGPPGPPGPC

SEQ ID NO: 129 - protein, 63 aa, Artificial
  synthetically constructed peptide of collagen III toolkit
GPCGPPGPPGPPGPPGPPGIKGHRGFPGNPGAPGSPGPAGQQGAIGPPGPPGPPGPPGPPGPC

SEQ ID NO: 130 - protein, 63 aa, Artificial
  synthetically constructed peptide of collagen III toolkit
GPCGPPGPPGPPGPPGPPGPAGQQGAIGSPGPAGPRGPVGPSGPPGPPGPPGPPGPPGPPGPC

SEQ ID NO: 131 - protein, 63 aa, Artificial
  synthetically constructed peptide of collagen III toolkit
GPCGPPGPPGPPGPPGPPGPVGPSGPPGKDGTSGHPGPIGPPGPRGPPGPPGPPGPPGPPGPC

SEQ ID NO: 132 - protein, 63 aa, Artificial
  synthetically constructed peptide of collagen III toolkit
GPCGPPGPPGPPGPPGPPGPIGPPGPRGNRGERGSEGSPGHPGQPGPPGPPGPPGPPGPPGPC

SEQ ID NO: 133 - protein, 63 aa, Artificial
  synthetically constructed peptide of collagen III toolkit
GPCGPPGPPGPPGPPGPPGERGSEGSPGHPGQPGPPGPPGAPGPCGPPGPPGPPGPPGPPGPC

SEQ ID NO: 134 - protein, 36 aa, Artificial
  synthetically constructed (GPP)10
GPCGPPGPPGPPGPPGPPGPPGPPGPPGPPGPPGPC

SEQ ID NO: 135 - protein, 36 aa, Artificial; identical to SEQ ID NO: 134
  synthetically constructed CRP

SEQ ID NO: 136 - protein, 36 aa, Artificial; identical to SEQ ID NO: 134
  synthetically constructed (GPO)1

SEQ ID NO: 137 - protein, 36 aa, Artificial; identical to SEQ ID NO: 134
  synthetically constructed (GPO)2

SEQ ID NO: 138 - protein, 36 aa, Artificial; identical to SEQ ID NO: 134
  synthetically constructed (GPO)4

SEQ ID NO: 139 - protein, 36 aa, Artificial; identical to SEQ ID NO: 134
  synthetically constructed (GPO)6